(19) United States
(12) Patent Application Publication
(10) Pub. No.: US 2025/0382211 A1
(43) Pub. Date: Dec. 18, 2025

(54) BIOLOGICALLY INOCULATED SORPTIVE BEAD MEDIA

(71) Applicant: University of Iowa Research Foundation, Iowa City, IA (US)

(72) Inventors: Gregory H. LeFevre, Iowa City, IA (US); Debojit S. Tanmoy, Iowa City, IA (US)

(21) Appl. No.: 19/236,597

(22) Filed: Jun. 12, 2025

Related U.S. Application Data
(60) Provisional application No. 63/659,239, filed on Jun. 12, 2024.

(51) Int. Cl. C02F 3/34; C02F 3/00; C02F 101/38; C02F 103/00; C12N 1/14; C12R 1/645

(52) U.S. Cl. CPC C02F 3/347; C02F 3/348; C12N 1/145; C02F 2003/003; C02F 2101/38; C02F 2103/001; C02F 2103/002; C02F 2305/06; C12R 2001/645

(57) ABSTRACT

A biologically active sorptive media is disclosed for sustainable contaminant removal in, for example, stormwater management systems. This technology addresses the technical problem of hydrophilic trace organic contaminants and dissolved-phase nutrients passing through conventional stormwater infrastructure, posing risks to groundwater and surface water quality. The biologically active sorptive media comprises a biopolymer matrix formed from alginate hydrogel, encapsulating sorbent materials (e.g., powdered activated carbon (PAC), iron-based water treatment residuals (FeWTR]), growth substrates (e.g., wood flour), and biodegrading organisms (e.g., white rot fungi). These beads enable rapid sorption of contaminants during storm events and subsequent biodegradation during inter-storm periods, renewing sorption capacity. The beads are mechanically robust, scalable, and stable for extended periods in high ionic strength solutions. Applications include bioaugmentation of green stormwater infrastructure (GSI), wastewater treatment, and bioremediation. This approach transforms GSI science by coupling sorption and biodegradation for enhanced contaminant removal.

### PRIORITY

This application claims the benefit of the filing date of U.S. Application No. 63/659,239, filed on Jun. 12, 2024, the disclosure of which is incorporated by reference herein.

### GOVERNMENT SUPPORT

This invention was made with government support under 1844720 awarded by the National Science Foundation. The government has certain rights in the invention.

### BACKGROUND OF THE INVENTION

Dissolved contaminants in stormwater are increasingly recognized for their deleterious impacts to water quality (LeFevre et al. 2015). One major challenge in green stormwater infrastructure (GSI) systems such as bioretention is that hydrophilic, polar trace organic contaminants (TOrCs) can pass through conventional high-rate infiltration media, as was highlighted in a recent review (Spahr et al 2020, ES:WRT). TOrCs that are not removed can then potentially impact receiving surface waters that GSI is aiming to protect, as well as underlying groundwater. Amending bioretention systems with sorptive materials such as black carbon is one approach to improving contaminant retention; however, sorption capacity can eventually be exhausted.

### SUMMARY

Hydrophilic trace organic contaminants and dissolved-phase nutrients can pass through traditional stormwater management systems and create risk of groundwater contamination. Biologically active sorptive media can rapidly capture many stormwater-relevant pollutants and bio-transform these contaminants to sustain contaminant removal. Provided herein is a novel scalable media, BioSorp Beads, that can bioaugment contaminant-degrading microorganisms in stormwater infrastructure and have high pollutant sorption potential.

In one embodiment, the disclosure provides a biosorption bead comprising a biopolymer matrix formed from an alginate hydrogel. The matrix incorporates a sorbent material dispersed therein, a growth substrate, and a biodegrading organism encapsulated within the matrix. In some embodiments, the sorbent material can include activated carbon, an iron-based water treatment residual, powdered biochar, aluminum-based water treatment residuals, iron oxide coated sorbents, ion exchange media, zinc oxide coated sorbents, manganese oxide coated sand, or any combination thereof, with the activated carbon optionally being powdered activated carbon. The growth substrate may comprise wood flour, mulch or wood chips, corn or corn cob, shredded straw, grass, newspaper, cotton, rice husk, chlorella, or a combination thereof. Further, the biosorption bead may include an electron shuttle, such as anthraquinone-2,6-disulfonate. The biodegrading organism can be a fungus, including white-rot fungi like Trametes versicolor or P. ostreatus, or a bacterium, such as denitrifying or nitrifying bacteria, with stability maintained for at least three months at room temperature. Additionally, the alginate hydrogel may be a cation alginate, for example sodium alginate, and may be crosslinked, for instance with calcium ions or ferric ions such as CaCl₂) or FeCl₃. In certain embodiments, the biosorption beads are stable when soaked in liquid for about 8 to 12 months (e.g., approximately 10 months) and may further comprise one or more micronutrients or vitamins, such as vitamin B12, and are configured to sorb at least about 20 mg of dissolved contaminants per gram of bead.

In another embodiment, the disclosure provides a method for coupling the sorption of a dissolved contaminant from a liquid with subsequent biodegradation. In this method, a contaminated liquid is contacted with the biosorption beads, whereby at least one dissolved contaminant is removed from the liquid. In some embodiments, the biodegrading organism within the bead biodegrades the contaminant, thereby renewing the bead's sorption capacity. The contaminant can be a trace organic contaminant or phosphate, and the contaminated liquid may be runoff, wastewater, grey water, or stormwater.

### BRIEF DESCRIPTION OF THE DRAWINGS

The drawings illustrate generally, by way of example, but not by way of limitation, various embodiments discussed herein.

FIG. 1 provides a schematic diagram of BioSorp Bead making procedure. The final beads containing the encapsulated materials and culture are approximately 3 mm in diameter and hard to the touch. (FeWTR-iron water treatment residual; PAC-powdered activated carbon, AQDS=Anthraquinone-2,6-disulfonate.)

FIGS. 2A-2C demonstrate the effects of systematically varying bead compositions on the surface area and the total pore volume of different BioSorp Beads. Compositions: varied sodium alginate (SA) concentration (crosslinker concentration was constant), varied crosslinker concentration (sodium alginate concentration was constant; 3% CaCl₂)=270.3 mM CaCl₂); 5% CaCl₂)=450.5 mM CaCl₂)), varied crosslinker type (equimolar [270.3 mM CaCl₂)] concentrations of CaCl₂) and FeCl₃ (270.3 mM); sodium alginate concentration was constant], beads including/excluding AQDS (both sodium alginate and crosslinker concentrations were constant). (b) BET adsorption-desorption isotherms for PAC, Fe beads, and Ca beads. (c) Mechanical strengths (Young's modulus measurements) of Fe³⁺ alginate beads and Ca²⁺ alginate beads (n=5; multiple measurements made on 5 beads of each type; p-value calculated from non-parametric Mann-Whitney rank sum test).

FIG. 3 provides the effects of varying bead compositions on the bead surface (Scanning Electron Microscope [SEM] image scale bar=50 μm). Increasing sodium alginate concentration smoothed the bead surface (crosslinker concentration was constant). Increasing the crosslinker concentration (3% to 5% CaCl₂)) or changing the crosslinker to FeCl₃ from CaCl₂) also smoothed the bead surface (sodium alginate concentration was constant). Large cracks and pores were evident in beads crosslinked with FeCl₃.

FIGS. 4A-4C show percent change in weight of calcium-alginate and ferric-alginate beads, kept in DI water, synthetic stormwater, and synthetic seawater (Bead swelling: Equation 1). Error bars represent the standard deviation of the mean (n=2 experimental replicates; Error bars too small to be visible are obscured by the data points). (c) Ferric-alginate beads and Calcium-alginate beads do not disintegrate in synthetic stormwater (ionic strength=0.005 M), 10× synthetic stormwater (ionic strength=0.046 M), 20× synthetic stormwater (ionic strength=0.093 M) even after 10 months.

FIG. 5 provides dissolved calcium concentration (leaching from Ca Beads) and dissolved iron concentration (leaching from Fe Beads) in solutions with different ionic strength (n=1). 10× synthetic stormwater, 20× synthetic stormwater, and synthetic seawater data could not be collected on day 42.

FIGS. 6A-6D demonstrate that white rot fungi can grow from the BioSorp Beads, demonstrating viability following drying and extended storage periods (i.e., 3 months). (a) and (b) Trametes versicolor grew from the BioSorp Beads (made with 1% sodium alginate, 1% powder activated carbon, 1% wood flour, and 3% CaCl₂)) when the beads were kept in malt extract media, (c) closeup stereoscope image of T. versicolor growth from BioSorp Beads (made with 1% sodium alginate, 1% powder activated carbon, 1% wood flour, and 3% CaCl2), (d) closeup stereoscope image of T. versicolor growth from BioSorp Beads (made with 1% sodium alginate, 1% powder activated carbon, 1% wood flour, 1% iron water treatment residual, and 3% CaCl₂)).

FIG. 7. Nitrate sorption in synthetic stormwater via different abiotic beads. No removal occurs when there is no anion exchange media added to the beads, thus this material is critical for nitrate removal.

FIG. 8. Nitrate sorption in DI water and in synthetic stormwater via different abiotic beads.

FIG. 9. Nitrate removal in synthetic stormwater via the proposed BioSorp Bead (initial concentration=˜ 10 mg/L NO₃⁻—N).

FIG. S1. Calcium alginate beads (made with sodium alginate, PAC, wood flour, Fe-WTR, calcium chloride) (left) and Iron (III) alginate beads (made with sodium alginate, PAC, wood flour, Fe-WTR, Ferric chloride) (right). Final dried beads (˜3 mm) decrease in diameter by approximately half from the wet size; coin shown for size reference is a US Quarter.

FIG. S2. Powdered iron water treatment residuals (Fe-WTR).

FIG. S3. Mixture of sodium alginate, PAC, wood flour, Fe-WTR, and fungi.

FIG. S4. BioSorp Bead making process. PAC, wood flour, Fe-WTR, and white rot fungi culture are mixed thoroughly in a beaker (left). The mixture is then pumped using a peristaltic pump and dropped into a crosslinking solution (in this case, calcium chloride) (right). The crosslinking solution is continuously shaken on a platform shaker such that the formed beads do not stick to each other.

FIG. S5. Bead formation in calcium chloride solution. The wet beads are approximately 6 mm in diameter.

FIG. S6. Wet BioSorp Beads being air-dried on wax paper.

FIG. S7. Dried BioSorp beads in palm of the hand (left) and on wax paper (right).

FIG. S8. Making of PAC-wood flour beads with sodium alginate and ferric chloride.

FIG. S9. Air drying PAC-wood flour beads on wax paper.

FIG. S10. Precipitated iron from Ferric-alginate beads in synthetic seawater.

FIG. S11A-S11C. BET adsorption isotherms for three types of beads-(a) 0.5% sodium alginate-1% PAC-1% wood flour-3% CaCl₂), (b) 1.5% sodium alginate-1% PAC-1% wood flour-3% CaCl₂), and (c) 1% sodium alginate-1% PAC-1% wood flour-5% CaCl₂.

FIG. S12. BET adsorption isotherms for two types of Ca beads-type 1: contains AQDS (1% sodium alginate-1% PAC-1% wood flour-1% FeWTR-3% CaCl₂)) and type 2: does not contain AQDS (1% sodium alginate-1% PAC-1% wood flour-1% FeWTR-3% CaCl₂)).

FIG. S13. SEM images of different composite alginate beads (scale bar=1 mm).

FIG. S14. Novel biologically active geomedia (BioSorp Beads) can remove trace organic contaminants from synthetic stormwater runoff via coupled sorption and biodegradation.

FIG. A1. (a) Imidacloprid, desnitro-imidacloprid, and acetanilide sorption kinetics for different types of BioSorp Beads. (n=3 experimental replicates; Error bars represent standard errors of the means. Some error bars are obscured by the data points as the error bars are small). The bead compositions are shown in Table SI 1.

FIGS. A2A-A2B. (a) Neonicotinoid sorption isotherms (sorbent: PAC_WF_CaCl2 beads). (n=3 experimental replicates; Error bars represent standard errors of the means. Some error bars are obscured by the data points as the error bars are small), (b) Neonicotinoid maximum sorption capacities of different BioSorp Beads and the maximum sorption capacities when normalized to the amount of PAC present in the beads. The bead compositions are shown in Table SI 1. ** sorption capacities were predicted from Modified Langmuir kinetic model. * PAC maximum sorption capacities are taken from Webb et al. (2020).

FIG. A3. Effects of varied alginate concentration, varied crosslinker (calcium chloride) concentration, and varied drying temperature of different BioSorp Beads on imidacloprid and desnitro-imidacloprid sorption kinetics. (n=3 experimental replicates; Error bars represent standard errors of the means. Some error bars are obscured by the data points as the error bars are small).

FIGS. A4A-A4B. Phosphate sorption kinetics for different types of alginate beads (left: Ca-beads and right: Fe-beads). Compositions of the beads are shown in Table SI 1*. (n=3 experimental replicates; Error bars represent standard errors of the means. Some error bars are obscured by the data points as the error bars are small).

FIG. A5. Comparison of three different PFAS sorption onto different BioSorp Beads and other sorptive materials found in the literature. The values and references can be found on Table SI 1.

FIG. A6. Coupled sorption and biodegradation of acetanilide via BioSorp Beads (different types of composite Ca²⁺ alginate and Fe³⁺ alginate beads) (initial acetanilide concentration=˜ 40 mg/L) (n=3 experimental replicates. Error bars represent standard errors of the means. Some error bars are obscured by the data points as the error bars are small).

FIG. SI 1. Imidacloprid and desnitro-imidacloprid sorption onto different beads.

FIG. SI 2. Imidacloprid (left) and desnitro-imidacloprid (right) sorption on PAC-wood dust calcium alginate beads.

FIG. SI 3. PFAS sorption onto different materials.

FIG. SI 4. Imidacloprid sorption capacities of raw PAC and BioSorp Beads (n=3 experimental replicates; Error bars represent standard errors of the means. Some error bars are obscured by the data points as the error bars are small). Here, PAC=Powdered activated carbon, WF=Wood flour, WTR=Iron water treatment residuals.

### DETAILED DESCRIPTION OF THE INVENTION

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

Provided herein are composition and methods for coupling rapid initial chemical sorption during storm events with subsequent biodegradation that is a transformative solution to sustainable contaminant removal in stormwater media. One embodiment provides for the facilitation of the rapid capture of chemical contaminants in stormwater during infiltration via sorption and then subsequently biodegrade contaminants during the inter-storm periods (which can renew sorption capacities of the media). This will effectively decouple the hydraulic residence time from the chemical contact time—in much the same way that activated sludge revolutionized wastewater treatment by decoupling Hydraulic Residence Time (HRT) from solids residence time.

As an example, urban stormwater runoff presents a significant environmental challenge, as the runoff often contains dissolved contaminants, including hydrophilic trace organic compounds (TOrCs) and dissolved-phase nutrients, which are challenging to remove using conventional stormwater management systems. Green stormwater infrastructure (GSI), such as bioretention cells, is commonly implemented to enhance water quality and support groundwater recharge. However, these systems generally rely on porous media like sand, compost, and soil, which are effective at removing particle-bound pollutants but tend to perform inadequately in capturing dissolved-phase contaminants. Hydrophilic TOrCs, such as pesticides, pharmaceuticals, and tire wear compounds, frequently pass through these systems, posing risks to groundwater and surface water quality. Furthermore, sorptive amendments like biochar or activated carbon can enhance contaminant retention but experience limitations in sorption capacity, which diminishes over time, requiring frequent replacement and maintenance. This highlights the need for a sustainable approach that not only captures contaminants but also renews sorption capacity to maintain effectiveness over extended periods.

The present disclosure addresses these limitations by providing a novel biologically active sorptive media, referred to as “BioSorp Beads,” which couples rapid sorption of contaminants with subsequent biodegradation to sustain contaminant removal. The described concept integrates a biopolymer matrix formed from alginate hydrogel, which encapsulates sorbent materials (e.g., powdered activated carbon (PAC), iron-based water treatment residuals (FeWTR)), growth substrates (e.g., wood flour), and biodegrading organisms (e.g., white rot fungi or denitrifying bacteria). During storm events, the beads rapidly capture contaminants via sorption, while the encapsulated microorganisms biodegrade the sorbed contaminants during inter-storm periods, effectively renewing the sorption capacity of the media. This approach decouples the hydraulic residence time required for stormwater infiltration from the chemical contact time needed for biodegradation, enabling sustained contaminant removal without compromising the hydraulic conductivity of GSI systems.

The BioSorp Beads are mechanically robust, scalable, and stable for extended periods, even in high ionic strength solutions such as synthetic seawater. Their physical properties, including surface area, pore volume, and mechanical strength, can be customized by adjusting the composition and crosslinking parameters. Additionally, the beads are designed to maintain the viability of encapsulated microorganisms for extended storage periods, making them practical for field applications. By combining sorption and biodegradation in a single media, this technology transforms GSI science and engineering practice, offering a sustainable and adaptable solution for stormwater management, wastewater treatment, and bioremediation applications.

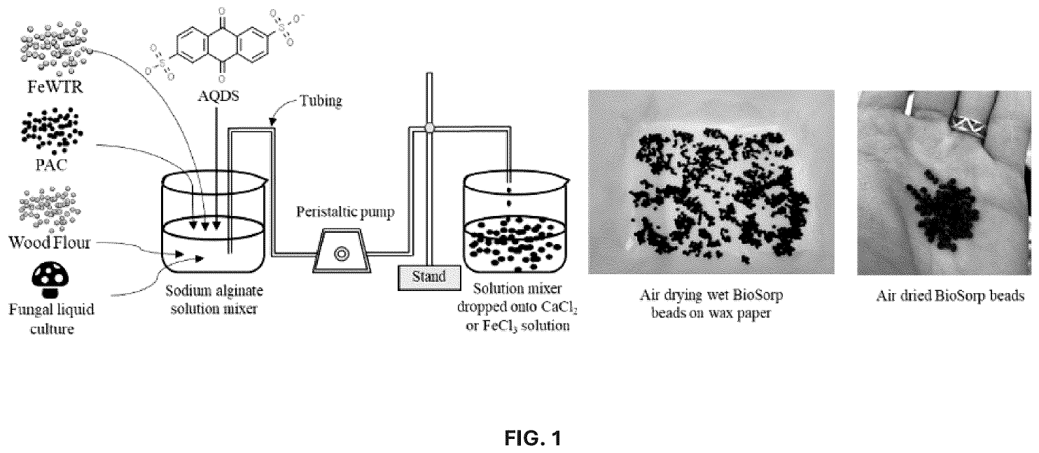

Provided herein is a geomedia that is a novel assemblage of materials (to rapidly capture a diverse suite of relevant dissolved stormwater contaminants) and encapsulate microorganisms (to bioaugment GSI and to enable contaminant biodegradation, renewing GSI sorption capacity). The inventors encapsulated powdered activated carbon [PAC] (sorbent), iron-based water treatment residual [FeWTR] (increased density, sorbent), wood flour [WF] (growth substrate), biodegrading organisms, and AQDS (model electron shuttle) in cation-alginate crosslinked matrices (either Ca²⁺ or Fe³⁺ crosslinkers). The final dried beads (“BioSorp Beads”) containing the encapsulated materials and culture are approximately 3 mm in diameter and physically hard to the touch (FIG. 1). The beads are made with economical, non-toxic, and commonly available materials to sorb contaminants and sustain organism viability, and enable recycling of waste products (i.e., iron water treatment residuals, wood flour). Different bead physical properties, such as surface area, pore volume, mechanical strength, swelling, and leaching can be tuned by adjusting composition. For instance, crosslinking with FeCl₃ vs. CaCl₂) increased bead mechanical strength, resulting in a media that is 36 to 850 times higher than the typical reported mechanical strengths of alginate beads. The beads are also physically durable, able to sustain long periods of soaking (up to 10 months), even in high ionic strength solution (synthetic seawater). The production of “BioSorp Beads” can easily be scaled for practical field application due to the use of inexpensive materials and simple production process.

Based on prior work (Wiener and LeFevre, 2022) where it was demonstrated that white rot fungi can biodegrade some types of emerging toxic tire wear compounds newly discovered to be ubiquitous in stormwater, white-rot fungi were chosen as the representative biodegrading organism that can bioaugment green stormwater infrastructure. Herein it was demonstrated that encapsulated biodegrading fungi can colonize from the beads when inoculated into growth media, thus indicating bioaugmentation. The fungi remain viable for extended periods (i.e., >3 months) even if beads are stored at room temperature (due to the encapsulated sustaining carbon source, i.e., wood flour), making the encapsulation approach practical for field applications.

One embodiment provides for deploying targeted biodegrading microorganisms, i.e., bioaugmentation specifically designed for GSI applications. Provided herein is a novel sorptive bioactive geomedia for bioaugmentation of Green Stormwater Infrastructure (GSI) that rapidly captures soluble chemical contaminants during infiltration and then subsequent biodegradation via encapsulated organisms. The compositions and methods provided herein transform GSI science and engineering practice.

Further, in other embodiments, however, the BioSorp Beads are an adaptable platform technology. Different bead physical properties can be tuned via materials adaptation according to the application needs, with the capability to encapsulate and deploy a variety of types of microorganisms for a suite of stormwater, wastewater, bioremediation, or other biotechnology applications (e.g., incorporation into fluidized bed bioreactor, Anammox bacteria, contaminated sediment in situ remediation).

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, several embodiments with regards to methods and materials are described herein. As used herein, each of the following terms has the meaning associated with it in this section.

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14th Edition, by R. J. Lewis, John Wiley & Sons, New York, N.Y., 2001.

For the purposes of clarity and a concise description, features can be described herein as part of the same or separate embodiments; however, it will be appreciated that the scope of the invention may include embodiments having combinations of all or some of the features described.

References in the specification to “one embodiment,” “an embodiment,” etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrase “one or more” is readily understood by one of skill in the art, particularly when read in context of its usage. For example, one or more substituents on a phenyl ring refers to one to five, or one to four, for example if the phenyl ring is di-substituted.

As used herein, “or” should be understood to have the same meaning as “and/or” as defined above. For example, when separating a listing of items, “and/or” or “or” shall be interpreted as being inclusive, e.g., the inclusion of at least one, but also including more than one of a number of items, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

The term “about” can refer to a variation of +5%, +10%, +20%, or +25% of the value specified. For example, “about 50” percent can in some embodiments carry a variation from 45 to 55 percent. For integer ranges, the term “about” can include one or two integers greater than and/or less than a recited integer at each end of the range. Unless indicated otherwise herein, the term “about” is intended to include values, e.g., weight percentages, proximate to the recited range that are equivalent in terms of the functionality of the individual ingredient, the composition, or the embodiment. The term about can also modify the endpoints of a recited range as discuss above in this paragraph.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to,” “at least,” “greater than,” “less than,” “more than,” “or more,” and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents.

One skilled in the art will also readily recognize that where members are grouped together in a common manner, such as in a Markush group, the invention encompasses not only the entire group listed as a whole, but each member of the group individually and all possible subgroups of the main group.

Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

Various methodologies of the instant invention include a step that involves comparing a value, level, feature, characteristic, property, etc. to a “suitable control,” referred to interchangeably herein as an “appropriate control” or a “control sample.” A “suitable control,” “appropriate control” or a “control sample” is any control or standard familiar to one of ordinary skill in the art useful for comparison purposes.

As used herein, the terms “including”, “includes”, “having”, “has”, “with”, or variants thereof, are intended to be inclusive similar to the term “comprising.”

The terms “comprises,” “comprising,” and the like can have the meaning ascribed to them in U.S. Patent Law and can mean “includes,” “including” and the like. As used herein, “including” or “includes” or the like means including, without limitation.

### Sorbent

A sorbent is a substance which has the property of collecting molecules of another substance (such as a contaminate) by sorption (a physical and chemical process by which one substance becomes attached to another). Sorbents can include, but are not limited to, carbon base products, perlite, vermiculite, glass wool, sand, volcanic ash, clay, peat, cellulose, sawdust, ground corn cobs, hay, feathers, polypropylene, polyurethane, polystyrene, epoxy, calcium carbonate, and magnesium carbonate. In one embodiment, the sorbent is activated carbon, including powdered activated carbon (PAC). In another embodiment, the sorbent is anion exchange resin. The sorbent can also include, but is not limited to, iron-based water treatment residual (FeWTR), powdered biochar, pyrolyzed black carbon, graphite/graphene including functionalized and non-functionalized carbon nanotubes (Webb et al. Environ. Sci. Technol. 2020, 54, 22, 14694-14705), aluminum-based water treatment residuals, iron oxide coated sorbents, powdered iron oxides, zinc oxide coated sorbents, manganese oxide coated sand or combinations thereof.

### Growth Substrate

As provided herein, a growth substrate provides nutrients to the biodegrading organism. Growth substrates can include, but are not limited to, wood flour (WF), mulch/wood chips, corn and/or corn cob, shredded straw, grass, cellulose, newspaper, cotton, rice husk, and chlorella.

### Electron Shuttle

Electron shuttles (ESs), also referred to as redox mediators, are organic molecules that can reversibly be oxidized and reduced, thereby conferring the capacity to serve as electron carriers among multiple redox reactions. ESs include, but are not limited to, 2-amino-3-carboxy-1,4-naphthoquinone, sodium 1,2-naphthoquinone-4-sulfonate (NQS), anthraquinone-2-sulfonate (AQS), anthraquinone-2-6-sulfonate (AQDS) and/or plant-derived 1,4-naphthoquinones, including juglone, plumbagin, and 1,4-naphthoquinone.

### Biodegrading Organisms

Biodegradation is the breakdown of matter by microorganisms. Biodegrading organisms can include bacteria, yeast and fungi, such as denitrifying bacteria, nitrifying bacteria and white rot fungi.

Nitrifying bacteria like Nitrosomonas and Nitrobacter convert ammonia into nitrites and nitrates. Denitrifying bacteria such as Thiobacillus denitrificans and Achromobacter convert nitrates back into nitrogen gas.

Bacteria for use herein can also include: 1) nitrogen removal bacteria-Anammox bacteria (bacteria that perform the anammox process are genera that belong to the bacterial phylum Planctomycetota; stands for “anaerobic ammonium oxidation,” nitrite and ammonium ions are converted directly into diatomic nitrogen and water); 2) PFAS degrading organisms that would thrive in the presence of iron and low pH, such as Acidimicrobium sp. Strain A6. (Huang and Jaffe, Environ. Sci. Technol. 2019, 53, 19, 11410-11419; Park et al. J. of Hazardous Materials. 2023, 495 132039); and/or 3) PCB degrading organisms such as Paraburkholderia xenovorans Strain LB400 (Dong et al. Environ. Sci. Technol. 2024, 58, 8, 3895-3907).

White rot fungi are a type of fungi comprising agaricomycetes, basidiomycetes, and some ascomycetes that are capable of decomposing many tree species. White-rot fungi are characterized by their ability to break down the lignin, cellulose, and hemicellulose of wood. As a result of this ability, white-rot fungi are considered a component of the carbon cycle, because of their ability to access carbon pools that would otherwise remain inaccessible. The name “white rot” derives from the white color and rotting texture of the remaining crystalline cellulose from wood degraded by these fungi. White rot fungi include, but not limited to, Coriolus versicolor, Phanerochaete chrysosporium, Phellimus pini, Phlebia spp., Cyathus stercoreus, Ceriporiopsis subvermispora, Pleurotus spp., Klebsiella pneumoniae and Ochrobactrum tritici, Trametes versicolor and P. ostreatus.

### Alginate

Alginate is a polysaccharide with the property of forming hydrogels, by ionic cross-linking with, for example, calcium ions or ferrous ions. Alginate can be obtained from marine algae and bacteria. The chemical structure is a copolymer of blocks, which are formed from β-D-manuronic acid (M) and α-L-guluronic acid (G) linked through 1,4-glucosidic bond. Its structure is heteropolymeric, that is, a combination of manuronic (M)/guluronic (G) residues, and its sequence varies according to the source from which it is obtained.

### Micronutrients

The biosorption beads provided herein can also include one or more micronutrients/vitamins. Micronutrients can include, but are not limited to, calcium, iron, sodium, zinc, copper, iodine, selenium, fluoride, magnesium, vitamin A, vitamin B12, vitamin C, sulfur, vitamin K, B, vitamins, folate, potassium, phosphorus, vitamin D, chloride, chromium, choline, and/or manganese.

### Liquid/Contaminates

In some embodiments, the liquid includes, but is not limited to, runoff water, stormwater, greywater, and/or wastewater.

In some embodiments, the contaminates to be removed include, but are not limited to, one or more trace organic contaminants (TOrCs, such as polycyclic aromatic hydrocarbons (PAHs), bisphenol analogs (BPs), polychlorinated biphenyls (PCBs), endocrine disrupting chemicals (EDCs), UV filters (UVs), organochlorine pesticides (OPs), pharmaceuticals and personal Care products (PPCPs), pesticides (including herbicides, insecticides, fungicides), tire-wear compounds, corrosion inhibitors or other vehicular fluids, per- and polyfluoroalkyl substances (PFAS) and/or antibiotics and antimicrobial compound). In some embodiments the contaminates are one or more of arsenic, copper, lead, disinfection byproducts, per- and polyfluoroalkyl substances (PFAS), mercury, nitrate, uranium, chromium, manganese, coliform, cryptosporidium, virus, fluoride, pesticide, radium, dioxane/dioxin, microorganism, perchlorate, radionuclide, radon, cadmium, carcinogenic VOCs, organohalogen compounds (such as organochlorines including PCBs and DDE) and other soluble chemicals.

### EXAMPLES

The disclosure can be better understood by reference to the following examples which are offered by way of illustration. The disclosure is not limited to the examples given herein.

Green Stormwater Infrastructure (GSI) is being increasingly implemented in urban areas as a nature-based solution to improve water quality and increase groundwater recharge. Nevertheless, GSI is inefficient at removing many trace organic contaminants (TOrCs) and dissolved-phase nutrients, risking groundwater contamination. Provided herein is engineered geomedia that rapidly captures stormwater pollutants via sorption, including TOrCs and dissolved nutrients, while bioaugmenting microorganisms to subsequently degrade captured contaminants in GSI. A novel “BioSorp Bead” bioretention geomedia was created by encapsulating powdered activated carbon (PAC) (sorbent), iron-based water treatment residual (FeWTR) (density, sorbent), wood flour (WF) (growth substrate), white-rot-fungi (WRF) (model biodegrading organism), and AQDS (model electron shuttle) in cation-alginate matrices (Ca²⁺, Fe³⁺). WRF culture was mixed with autoclaved PAC, FeWTR, AQDS, and WF in 1% alginate. This mixture was added dropwise via peristaltic pump into 270.3 mM CaCl₂) or FeCl₃ (on a platform shaker) to instantaneously form beads that were then air-dried. Encapsulated fungi remained viable in the dried beads over an extended period (3 months at room temperature), demonstrating usefulness in bioaugmentation applications. The bead physical properties were quantified (i.e., surface area, pore volume, mechanical strength, swelling, leaching), demonstrating that properties can be customized by adjusting composition parameters (e.g., crosslinking with FeCl₃ vs. CaCl₂) increased bead mechanical strength). The production process is economical, scalable, and uses non-toxic/recycled materials. The BioSorp Beads can facilitate rapid contaminant capture during infiltration of storm events and support microorganisms that subsequently degrade sorbed chemicals, thus renewing GSI sorption capacity in situ.

### Example 1—Composite Alginate Bead Media with Encapsulated Sorptive Materials and Microorganisms to Bioaugment Green Stormwater Infrastructure

### Introduction

Urban areas generate rapid and voluminous stormwater runoff during precipitation events, which contains complex mixtures of both dissolved phase and particle-bound contaminants that degrade water quality. (1) Conventional stormwater management methods are typically ineffective for removing dissolved phase contaminants (e.g., dissolved phosphorus [P], polar trace organics). (1-3) Green Stormwater Infrastructure (GSI) is being increasingly implemented as a nature-based solution to improve urban stormwater runoff quality, increase groundwater recharge, (4-7) and address broader societal needs. (8) Bioretention cells are one of the most widely used GSI practices. (3,9) Conventional bioretention cells are typically filled with porous media consisting of sand, compost, soil, and mulch with vegetation (10) to maintain high hydraulic conductivity/infiltration rates and prevent extending ponding. These cells successfully remove particle-associated pollutants, such as suspended solids, bacteria, some nutrients, and some heavy metals. (11) Nevertheless, dissolved phase compounds that are polar and hydrophilic, including many trace organic contaminants (TOrCs), demonstrate inferior removal in conventional media (11-13) and are more likely to pass through bioretention cells. (1) Failure to capture TOrCs in conventional bioretention media can potentially risk groundwater contamination. (14-16) Therefore, improved media for bioretention cells is necessary to remove TOrCs from stormwater in GSI while protecting underlying groundwater.

Amending conventional bioretention cell media with black carbon materials can substantially improve stormwater quality by rapidly capturing polar trace organic contaminants via sorption. Bioretention cells containing black carbon materials (i.e., biochar, granular or powdered activated carbon [GAC or PAC]), can help remove multiple different types of trace organics from stormwater runoff (17,18) via multiple sorption mechanisms. (19) For instance, Ulrich et al. (20) modified stormwater biofilters with biochar and achieved superior TOrC removal over traditional unamended biofilters. There are similar recent reports of improved high trace organic (21) and dissolved nutrient (22) removal in black carbon modified stormwater bioretention systems. Even amendment of black carbon in bioretention cells, however, does not represent a complete solution because sorption capacity can be exhausted over extended time periods. Thus, there is a growing need to develop improved media capable of degrading contaminants in situ to renew sorption capacities and sustain long-term pollutant removal of GSI while minimizing maintenance.

Enhancement of biological processes in GSI modified with black carbon could provide a sustained solution for the removal of captured TOrCs. Microbial uptake/metabolism can improve contaminant removal in bioretention cells by facilitating biologically mediated redox reactions during inter-storm periods, (23) which regenerates some of the GSI media sorption capacity. (24) Therefore, biologically active sorptive media holds the potential decouple the short hydraulic residence time in bioretention cell necessary for rapid stormwater infiltration from the longer chemical residence time needed for biodegradation of TOrCs, ensuring sustainable treatment. In addition to biodegradation, microorganisms further enhance contaminant removal via biotic sorption onto the biofilms. (24) Hence, biodegradation of TOrCs is a key to improvement of stormwater runoff quality though in situ renewal of sorption capacities of the treatment media. (25)

Studies related to contaminant biotransformation in GSI to date mainly focus around bacterial and plant processes, (26-28) while fungi are under-investigated in stormwater bioretention. White rot fungi (WRF) are a class of well-known wood decaying fungi that can produce a variety of both extracellular enzymes (e.g., manganese peroxidase (MnPs), lignin peroxidase (LiPs), laccases) and intracellular enzymes (e.g., CYP450) (29) are capable of degrading recalcitrant organic compounds. (30) It was recently reported (31) that WRF are capable of biodegrading some tire wear compounds, which are becoming increasingly recognized for their presence and persistence in urban stormwater and impacts to aquatic life. (32-35) WRF are also known to degrade urban-use recalcitrant pesticides (e.g., Trametes versicolor can degrade the phenylpyrazole-based pesticide fipronil (36)). When WRF are proximal to black carbon materials, biotransformation dynamics can change because the redox-active groups present on black carbon surfaces can function as redox mediators of fungal extracellular enzymes. (37) Black carbon materials can also immobilize fungal enzymes via adsorption onto the porous structures and by covalent bonds with the surface functional groups, resulting in higher fungal biodegradation potential. (38,39) Nevertheless, the limited research to date in bioretention has focused on fungal nutrient cycling or interactions with plants, with a distinct paucity of work on the potential to directly incorporated WRF into GSI systems. (40-42) Bioaugmenting GSI with WRF can play a role in trace organic biodegradation and improved stormwater quality, therefore representing a critical need.

Provided herein is a novel biologically active sorptive geomedia with encapsulated white-rot fungi to bioaugment green stormwater infrastructure. Here, the inventors encapsulated powdered activated carbon (PAC), wood flour, iron water treatment residuals (FeWTR), anthraquinone-2,6-disulfonate (AQDS), and Trametes versicolor as a model WRF in Ca²⁺/Fe³⁺ alginate hydrogel structures to create “BioSorp Beads”. The fungal viability in the dried beads was verified over an extended time period and a suite of BioSorp Bead physical properties was characterized (i.e., surface area, pore volume, mechanical strength, swelling, leaching etc.). Black carbon (PAC) has multiple active adsorption sites and is well-established to sorb various trace organics and ionic contaminants. (19) Iron oxides present in iron-based water treatment residuals (FeWTR), (43) can aid dissolved phosphorus and potential PFAS sorption. (44-46) Practically, FeWTR also increases the bead density to avoid floating media during precipitation events and maintains bioretention structural integrity. WRF secrete a variety of intra- and extracellular enzymes and degrade different trace organic compounds. (29, 31) Wood flour acts as a carbon/energy source for fungi to maintain fungal viability for extended time periods. (47) AQDS (a commonly used model electron shuttle) can enhance the degradation of various recalcitrant organic contaminants by acting as redox mediators for microbial metabolism/contaminant transformation. (48) This is the first study to integrate the potentials of deploying composite fungi alginate beads in bioretention cells for the goals of field bioaugmentation and subsequent trace organic removal from urban stormwater runoff, thus representing a novel assemblage of materials and organisms. The BioSorp Beads are a platform technology that can be adapted to encapsulate other microbes or materials for a suite of environmental remediation (e.g., contaminated sediment bioaugmentation) or biotechnology applications (e.g., fluidized bed bioreactor).

### Materials and Methods

### Chemicals, Materials, and Cultures.

Calcium chloride, ferric chloride, sodium alginate, sodium nitrate, magnesium chloride hexahydrate and powdered activated carbon (PAC) were purchased from Fisher Scientific. Anthraquinone-2,6-disulfonate (AQDS), sodium sulfate, and sodium bicarbonate were purchased from Sigma Aldrich. Synthetic seawater was prepared by mixing 36 g “Instant Ocean Sea Salt” (TopDawg Pet Supply, USA) per liter of deionized water. Ferric sludge containing iron water treatment residuals (FeWTR) was recovered from the University of Iowa water treatment plant. The ferric sludge was settled at room temperature for 24 hours. The top clear water layer was carefully removed via peristaltic pump to retain the bottom thickened FeWTR slurry. The remaining FeWTR slurry was first oven dried at 70° C. for 2 days, and then powdered using mortar and pestle. The wood flour (sanding dust residual; also known as wood dust) was purchased from Shannon's Sawmill (Syracuse, New York, USA). Malt extract broth, ammonium chloride, and sodium phosphate dibasic were purchased from Research Products International. Dr. Jordyn Wolfand from the University of Portland graciously provided the white rot fungi (Trametes versicolor) culture.

### Synthetic Stormwater

Synthetic stormwater was prepared using a previously described method dissolving 0.072 mM of NH₄Cl, 0.75 mM of CaCl₂, 0.33 mM of Na₂SO₄, 0.072 mM of NaNO₃, 1 mM of NaHCO₃, 0.075 mM of MgCl₂, and 0.016 mM of Na₂HPO₄ in deionized water (31).

### UIowa Water Treatment Plant

UIowa-WP uses Iowa river water as an influent and distributes 900 million gallons of water annually. The basic water treatment process in UIowa-WP includes but not limited to powdered activated carbon treatment, coagulation (currently using ferric chloride as coagulant), flocculation, sedimentation, lime softening, recarbonation, gravity filtration, and reverse osmosis. BioSorp Bead Preparation Overview Summary.

Provided here is a summary of the development of the novel sorptive bioactive ‘BioSorp Beads’ to amend bioretention cell media for microorganism delivery and trace organic removal from stormwater runoff via coupled sorption and biodegradation (FIG. 1). The materials are combined, and organisms encapsulated using alginate as a biopolymer. Alginate is a commonly used sorbent in wastewater treatment for heavy metal and ionic dye removal (49, 50) and can also be used for biofilm encapsulation and applied for bioaugmentation of GSI. Alginic acid is a natural organic polymer (anionic polysaccharide), which is found in brown algae. (51) This polymer contains abundant free hydroxyl and carboxyl groups in the polymeric chain that form irreversible crosslinking bonds with polyvalent cations (51) (e.g., Ca²⁺, Fe³⁺, Al³⁺). Calcium alginate beads form a two-dimensional ‘egg-box’ shaped structure, whereas Fe³⁺ alginate beads have a three-dimensional structure and are more porous than Ca²⁺ alginate beads. (52)

Different variations of BioSorp Beads were prepared using powdered activated carbon (PAC), wood flour (WF), iron water treatment residuals (FeWTR), AQDS, and T. versicolor culture to probe the effects of varying material properties (Table S1). These materials were thoroughly mixed in varied concentrations of sodium alginate solution (0.5% to 1.5% w/v in DI water) and then added dropwise into calcium chloride (270.3 mM or 450.5 mM tested; described in detail below) or ferric chloride (270.3 mM) solution (made in DI water) using a peristaltic pump. In every 100 mL of sodium alginate solution, 1 g of PAC, 1 g of WD, and/or 1 g of FeWTR were added. PAC, wood flour, water treatment residuals, and/or fungi cells are trapped in the cross-links to form the final BioSorp Beads. During the preparation process, the beakers containing calcium chloride or ferric chloride solutions were maintained on a platform shaker and constantly shaken at 50 rpm. Wet beads were then air dried on wax paper at room temperature for two to three days before being stored at room temperature. Dried beads (2.5 to 3 mm diameter) decrease in size by approximately 50% in diameter compared to wet beads (5 to 5.5 mm diameter).

TABLE S 1
Testing the impacts of different BioSorp Bead production conditions in various measured properties

| | | Bead Preparation Recipe | | | | | | | | | |
|---|---|---|---|---|---|---|---|---|---|---|---|
| | | | | | External | | | | | | Descriptive |
| | | Sodium | Cross- | Cross- | Electron | | | | | | Impacts on |
| Testing Conditions for | | Alginate | linker | linker | Shuttle | | Wood | Fe- | Drying | Measured | Measured |
| characterization | | Concentration | Concentration | Type | (AQDS) | PAC | Flour | WTR | Temperature | Bead Properties | Properties |
| 1. Varied | Baseline | 1% | 270.3 | CaCl₂ | — | 1% | 1% | — | Air dried | Surface | Surface area |
| Alginate | Conditions | | mM | | | | | | at room | area; Total | and pore |
| Concentration | | | | | | | | | temp | pore | volume |
| | Other | 0.5%, | 270.3 | CaCl₂ | — | 1% | 1% | — | Air dried | volume | decreases |
| | Conditions | 1.5% | mM | | | | | | at room | | with the |
| | | | | | | | | | temp | | increase in |
| | | | | | | | | | | | alginate |
| 2. Varied | Baseline | 1% | 270.3 | CaCl₂ | — | 1% | 1% | — | Air dried | Surface | Surface area |
| Crosslinker | Conditions | | mM | | | | | | at room | area; Total | and pore |
| Concentration | | | | | | | | | temp | pore | volume |
| | Other | 1% | 270.3 | CaCl₂ | — | 1% | 1% | — | Air dried | volume | decreases |
| | Conditions | | mM | | | | | | at room | | with the |
| | | | | | | | | | temp | | increase in |
| 3. Varied | Baseline | 1% | 270.3 | CaCl₂ | — | 1% | 1% | — | Air dried | Surface | Crosslinking |
| Crosslinker | Conditions | | mM | | | | | | at room | area; Total | with FeCl₃ |
| Type | | | | | | | | | temp | pore | increases |
| | Other | 1% | 270.3 | FeCl₃ | — | 1% | 1% | — | Air dried | volume | surface area |
| | Conditions | | mM | | | | | | at room | | and pore |
| | | | | | | | | | temp | | volume |
| 4. Effects | Baseline | 1% | 270.3 | CaCl₂ | — | 1% | 1% | 1% | Air dried | Surface | Addition of |
| of External | Conditions | | mM | | | | | | at room | area; Total | AQDS |
| Electron | | | | | | | | | temp | pore | marginally |
| Shuttle | Other | 1% | 270.3 | CaCl₂ | 0.1% | 1% | 1% | 1% | Air dried | volume | decreases |
| | Conditions | | mM | | | | | | at room | | surface area |
| | | | | | | | | | temp | | and pore |
| 5. Effects | Baseline | 1% | 270.3 | CaCl₂ | 0.1% | 1% | 1% | 1% | Air dried | Mechanical | Crosslinking |
| of Cross- | Conditions | | mM | | | | | | at room | Strength | with FeCl₃ |
| linker Type | | | | | | | | | temp | | increases |
| on | Other | 1% | 270.3 | FeCl₃ | 0.1% | 1% | 1% | 1% | Air dried | | bead |
| Mechanical | Conditions | | mM | | | | | | at room | | strength |
| | | | | | | | | | temp | | |
| 6. Drying | Baseline | 1% | 270.3 | CaCl₂ | 0.1% | 1% | 1% | 1% | Air dried | Fungal | Similar |
| Temperature | Conditions | | mM | | | | | | at room | Viability | fungal |
| | | | | | | | | | temp | | viability |
| | Other | 1% | 270.3 | CaCl₂ | 0.1% | 1% | 1% | 1% | Oven | | |
| | Conditions | | mM | | | | | | dried (at | | |
| | | | | | | | | | 70° C. for | | |
| | | | | | | | | | 8 hours) | | |

To encapsulate fungi within the BioSorp Beads, bead materials (i.e., PAC, wood flour, and FeWTR) were autoclaved at 121° C. for 60 minutes prior to starting the bead preparation steps. DI water and 70% ethanol were flushed through the peristaltic pump tubing to clean any residue. One week before preparing the beads, the fungal strain was freshly inoculated by transferring into an Erlenmeyer flask containing malt extract media from an agar plate using a sterile loop. The Erlenmeyer flask was maintained on a shaker table for one week to allow sufficient fungal growth (OD600˜1.5). After one week, the fungi culture was homogenized using a handheld OMNI homogenizer. In every 50 mL of 2% w/v sodium alginate solution, 50 mL homogenized fungi culture (containing fungi in malt extract media), 1 g PAC, 1 g WF, 1 g FeWTR, and 0.1 g AQDS was added. This mixture was thoroughly homogenized using the OMNI homogenizer to yield a 1% w/v final sodium alginate concentration in the mixture). Lastly, this mixture was added dropwise into 270.3 mM cationic (calcium chloride or ferric chloride) solution using the peristaltic pump to form the fungi containing beads. The beads were then air dried on wax paper at room temperature for two to three days.

### Bead Characterization Experimental Design.

A suite of abiotic beads (i.e., did not contain any fungal culture) were created to isolate and systematically vary and characterize the impacts of different preparation techniques on physical properties of the beads (i.e., varied sodium alginate concentration [0.5% to 1.5%], varied crosslinker concentration (3% vs 5% CaCl₂)), divalent/trivalent crosslinker type (CaCl₂) vs FeCl₃), with vs. without AQDS). The systematic bead experimental design and the impacts on different measured physical properties are described in Table 1 and Table S1. The bead surface area, pore volume, and mechanical strength were quantified. The bead surface was imaged using scanning electron microscopy (SEM). To examine the bead stability in solutions with different ionic strengths, the bead swelling ratio (Equation 1) was used and bead crosslinker leaching (i.e., dissolved calcium and iron concentration) in deionized water, synthetic stormwater, and synthetic seawater. We maintained 100 mg of dried Fe³⁺ crosslinked alginate beads and calcium crosslinked alginate beads in 50 mL of the aforementioned solutions at room temperature, and periodically weighed the wet beads to quantify bead swelling (Bead Swelling). The dissolved phase calcium or iron concentrations in the solutions were also periodically measured.

$$Bead swelling = \frac{wet bead weight - dried bead weight}{dried bead weight} \times 100 \%$$

Two types of biologically active BioSorp Beads were prepared (containing fungi; air dried beads and oven dried beads (dried at 70° C. for 8 hours)) and the finished beads were stored at room temperature in sterile sealed tubes. The oven dried beads were prepared to determine if drying temperature impacted fungal viability. To verify the viability of the encapsulated fungi, 250 mL growth media and 2 g of freshly prepared dried BioSorp Beads were combined and maintained on a platform shaker at 100 rpm for two weeks, whereupon the wet beads (and drops of the growth media) were imaged with an optical stereoscope to observe the fungal growth. The viability experiment was also repeated with beads stored at room temperature for three months.

### Analytical Methods

Bead surface area and porosity were measured using a Quantachrome Nova 4200e BET instrument and bead mechanical strength was measured using a molecular force probe 3D classic (MFP-3D) atomic force microscope. Dissolved calcium and dissolved iron concentrations were measured using an Agilent 7900 ICP-MS. The bead surface was measured using Hitachi S-4800 scanning electron microscope and Olympus SZX12 Stereoscope. A more detailed description of the analytical methods can be found herein below. All fungal culture work was conducted in a laminar flow biosafety cabinet (BSC). The BSC was decontaminated before and after each usage with 10% (v/v) bleach, 70% (v/v) ethanol, and UV-sterilization. Any solution or culture not immediately needed was stored in a refrigerator at 4° C. for later usage. GraphPad Prism 9.0.0 (San Diego, CA) was used to perform all statistical analyses. Normality of the data distribution was evaluated using the Shapiro-Wilk test and normal QQ plot. When the data were significantly different from a normal or log-normal distribution (α=0.05), non-parametric analysis was performed (e.g., Mann-Whitney rank sum test rather than t-tests). ANOVA (α=0.05) with Tukey post hoc tests were performed to quantify statistical significances among matched-paired datasets.

Bruanauer-Emmett-Teller (BET): A Quantachrome Nova 4200e BET instrument was used to determine the bead surface area and pore volume. All the samples were analyzed by the University of Iowa MATFab Facility (matfab.research.uiowa.edu), and the collected data were analyzed using the NovaWin software. BioSorp Bead samples were first inserted into glass tube sample holders with rod inserts and were then degassed at 90° C. for 15 hours. The sample mass was recorded for later analysis. During anylysis, the glass tube containing the samples was immersed in liquid nitrogen and a known quantity of ultra-pure nitrogen was introduced by the instrument for the nitrogen adsorption test. The bead surface area and the pore volume were quantified using the following BET equations 953) (Equation 1, Equation 2):

$$\frac{x}{V ( 1 − x )} = \frac{1}{( {V}_{m} ) ( {C}_{BET} )} + \frac{x ( {C}_{RET} − 1 )}{( {V}_{m} ) ( {C}_{BET} )} \qquad Equation 1$$

where V=volume of the adsorbed molecules, Vm=monolayer, C_{BET}=BET constant, x=relative pressure (P/P0).

$$Surface area = \frac{( V {□}_{m} ) ( {N}_{A} ) ( {a}_{m} )}{( {V}_{ms} ) ( {m}_{s} )} \qquad Equation 2$$

where N_{A}=Avogadro's number (6.022×10²³ mol-1), aₘ=effective cross-section area of one adsorbed molecule, vₘ=molar volume of one adsorbed molecule, mₛ=mass of the adsorbent.

Atomic Force Microscope (AFM): Stiffness of the beads was quantified using a Molecular Force Probe 3D Classic (MFP-3D) Atomic Force Microscope (Asylum Research, Santa Barbara, CA, USA). Young's moduli were measured on multiple positions for each bead to obtain the representative average bead mechanical strength. All AFM studies were conducted by the Tivanski group at the Department of Chemistry, University of Iowa and using previously established AFM protocols. (54-56) All AFM experiments were performed at room temperature and pressure. The force analysis was conducted using MikroMasch CSC37 silicon nitride probes (nominal spring constant=˜ 1.0 N/m; tip radius of curvature=10 nm; scan rate=1 Hz). Thermal noise method was used to determine the actual AFM tip spring constant. A humidity cell was used to control the relative humidity (RH) and an intermittent contact mode (AC mode) was used to generate the AFM height images at that specific RH. Whenever the RH changed, 10 to 15 minutes of equilibration time was allotted to ensure thermodynamic balance.

Scanning Electron Microscope (SEM), Stereoscope, and ICP-MS: Closeup surface images of the beads were taken using Hitachi S-4800 Scanning Electron Microscope. All the samples were run by the Central Microscopy Research Facility (CMRF, cmrf.research.uiowa.edu/) at the University of Iowa. Dried samples were placed on adhesive carbon coated aluminum stubs. The stubs were sputter-coated with gold and palladium (60:40) prior to the imaging steps. An Olympus SZX12 Stereoscope at the CMRF was used to image fungal growth from the beads. Agilent 7900 ICP-MS was used to measure dissolved phase Ca²⁺ and Fe³⁺ concentrations for the leaching experiment.

QA QC and Statistical Analysis: All fungal culture work was conducted in a laminar flow biosafety cabinet (BSC). The BSC was decontaminated before and after each usage with 10% (v/v) bleach, 70% (v/v) ethanol, and UV-sterilization. Fungal culture preparation and maintenance procedures were followed from our earlier work. (31) Any solution or culture not immediately needed was stored in a refrigerator at 4° C. for later usage. GraphPad Prism 9.0.0 (San Diego, CA) was used to perform all statistical analyses. Normality of the data distribution was evaluated using the Shapiro-Wilk test and normal QQ plot. When the data were significantly different from a normal or log-normal distribution (α=0.05), non-parametric analysis was performed (e.g., Mann-Whitney rank sum test rather than t-tests). ANOVA (α=0.05) with Tukey post-hoc tests were performed to quantify statistical significances among matched-paired datasets.

### Results and Discussion

### Summary Description of the Produced Geomedia.

The main objective was to develop sorptive engineered geomedia (BioSorp Beads) that can be used as vehicles for GSI bioaugmentation. The design was tailored to employ non-toxic materials, incorporate recycled/valorized waste products, and maintain an inexpensive production process that is scalable for practical applications to field stormwater management systems. The produced beads have an extended viable shelf-life for the encapsulated organisms (at least 3 months when stored at room temperature). Previous literature indicates that white rot fungi can remain highly viable in calcium alginate beads for at least one year when stored at 5° Celsius. (47) The BioSorp Beads can rapidly capture a spectrum of environmentally relevant dissolved stormwater pollutants and to bioaugment contaminant degrading microorganisms in GSI, thereby facilitating trace organic contaminant biodegradation while renewing the GSI's sorption capacities. Furthermore, the BioSorp Beads are mechanically robust and appear sufficiently stable to even sustain high ionic strength stormwater runoff (i.e., relevant in winter when high amounts of deicing salts are applied), as detailed below.

The final produced dried BioSorp Beads, designed for bioaugmentation and contaminant capture/bioremediation, are approximately 2.5-3 mm in diameter (FIG. 1). This physical size is desirable to maintain high hydraulic conductivity in GSI systems. The Ca²⁺ alginate beads are spherical when freshly prepared, whereas the Fe³⁺ alginate beads generate a more disk-like/spheroidal shape, indicating denser crosslinks due to the presence of trivalent iron ions. The ‘final’ beads all contain PAC (to rapidly capture different trace organics), white rot fungi (as a model organism to bioaugment the bioretention cells, enabling contaminant biodegradation), wood flour (to support and maintain fungal growth), AQDS (as a redox mediator), and FeWTR (to increase bead density as well as dissolved nutrient and PFAS removal). Regardless of composition, alginate beads crosslinked with calcium chloride had higher pH (around 6 to 6.5) than beads crosslinked with ferric chloride (around 3.3 to 3.6). The details of different bead properties and the bead geomedia potential to bioaugment green stormwater infrastructure are described below.

### Physical Properties of BioSorp Beads.

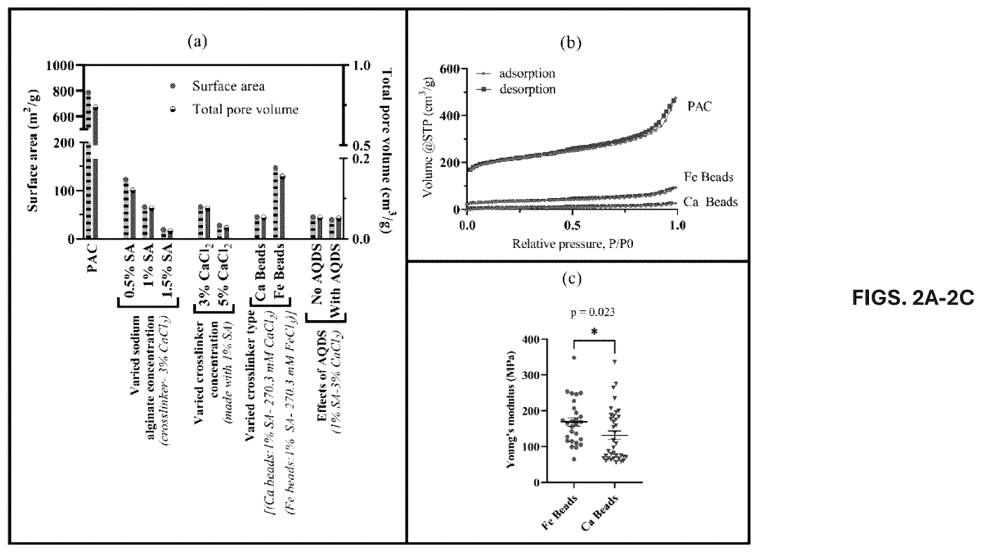

Effects of bead composition on surface area and pore volume: the inventors systematically probed the effects of alginate and cationic crosslinker concentration on bead surface area and porosity (Table 1 and FIG. 2). A substantial decrease was observed in surface area from the original PAC to the beads (approximately 5- to 43-fold decrease). This loss of surface area for the beads compared to the PAC is attributed to the encapsulation of PAC in the alginate gel structure and is entirely expected based on the literature. (57) Similar surface area decreases are also reported in other activated carbon hydrogel beads (57) (e.g., unencapsulated activated carbon surface area was 897.9 m²/g, whereas a chitosan bead containing the activated carbon had a surface area of 165.8 m²/g).

Both the surface area and pore volume of the BioSorp beads decreased with increasing alginate concentrations in the recipe, likely due to the denser crosslinked structures. (58) When the sodium alginate (SA) concentration of the beads was increased from 0.5% to 1%, the surface area decreased by 46%. Surface area further decreased when the alginate concentration was raised to 1.5%. Similar effects were also observed when the crosslinker concentrations increased. Beads made with 5% CaCl₂) (450.5 mM) had 57.9% lower surface area than the beads made with 3% CaCl₂) (270.3 mM). Beads created with equimolar ferric chloride (270.3 mM FeCl₃) crosslinker had a higher surface area (69.1% higher) and total pore volume (66.2% higher) than beads made with calcium chloride (270.3 mM CaCl₂)). Ferric-alginate beads have three-dimensional binding structures and calcium-alginate beads have two-dimensional planar binding structures. Therefore, it was hypothesized that the two-dimensional planar structures in calcium-alginate beads result in lower porosity than ferric-alginate beads.

TABLE 1
Table 1. Surface area and total pore volume of different calcium-alginate and ferric-alginate beads. The linking brackets
in the experimental design column highlight the testing of one variable while holding other factors constant.

| | | | Results | | |
|---|---|---|---|---|---|
| | | | | Total | |
| Experimental design | | | Surface | pore | |
| | Test | | area | volume | Effect Outcome |
| No | Conditions | Recipe | (m²/g) | (cm³/g) | Summary |
| 1 | | Raw PAC | 787.51 | 0.737 | |
| 2 3 4 5 | Varied Alginate concentra- tion Varied crosslinker concentra- tion | 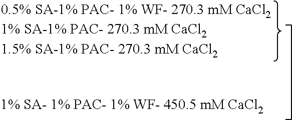 | 122.93 65.86 18.17 27.73 | 0.121 0.076 0.019 0.027 | Alginate Concentration ↑: Surface area↓; Pore Volume↓; Crosslinker Concentration↑: Surface area↓; Pore Volume↓; |
| 6 7 8 | Varied crosslinker type Effects of External Electron Shuttle | 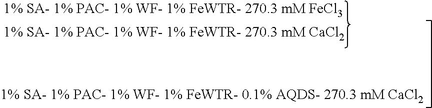 | 146.62 45.24 39.35 | 0.155 0.052 0.051 | FeCl₃ = ↑ Surface area and Pore Volume: CaCl₂ = ↓Surface area and Pore Volume; AQDS marginally decreases surface area and pore volume |

Hence, surface area and pore volumes can be customized by either adjusting the alginate concentration or the crosslinking concentration or the crosslinker type (FIG. 2(a)). It was concluded that the ferric cross-linked alginate beads may be better candidates for most GSI applications due to the higher porosity and surface area. When AQDS was added to the bead recipe (using calcium alginate as test beads), surface area and pore volume of the AQDS containing beads decreased marginally by 13% and 3.3%, respectively, compared to the beads that did not contain any AQDS. AQDS is a model quinone substance that can facilitate microbial and abiotic degradation of various recalcitrant trace organics by facilitating e-transfer. (59-62) Considering potential for the added benefits external electron shuttles provide, it was hypothesized that the presence of AQDS would likely have a net positive impact on the bead performance and AQDS.

As a quality assurance measure, the BET adsorption isotherms (volume of nitrogen gas adsorbed by PAC, ferric-alginate beads, and calcium-alginate beads as a function of relative pressure) were characterized, which were used to quantify bead surface areas and pore volumes (FIG. 2(b)). All isotherms followed IUPAC type IV (a) isotherms, where capillary condensation and hysteresis coexist. Capillary condensation (gas phase transitions into liquid phase) and hysteresis (adsorption and desorption pathway do not overlap) were observed in the alginate gel bead isotherms. The hysteresis loop follows loop type H4, where noticeable adsorption at low relative pressure indicates micropore filling. This type of isotherm occurs when mesopores larger than 4 nm are present in the structures. Type IV (a) isotherms and H4 loops are common in activated carbons (63) and oxide gels like zeolite. (64)

Effects of bead composition on mechanical strength: Fe³⁺ alginate beads possess higher mechanical strength than Ca²⁺ alginate beads (FIG. 2(c)). Mean values for the Young's modulus of the Fe³⁺ beads and Ca²⁺ beads were 169.7 MPa and 131.4 MPa, respectively (p=0.023). The Young's moduli of the BioSorp Beads (made with 1% alginate; encapsulated 1% PAC, 1% WF, 354 and 1% FeWTR) are 36 to 850 times higher than the typical reported (65,66) mechanical strengths of alginate beads containing no amendments (i.e., nothing encapsulated; beads made with 10% alginate concentration exhibited a Young's modulus of 3.6 Mpa (66), whereas the values for 2% to 5% alginate beads ranged from 0.2 MPa to 0.6 MPa). (65) Other organic hydrogel beads are reported to have even lower mechanical strength than alginate beads (e.g., 1.5% chitosan bead have a reported Young's modulus of 0.187 MPa). (67) This finding indicates that the bioretention cells can maintain structural integrity (i.e., not to subside) if BioSorp Beads were incorporated in the cell media.

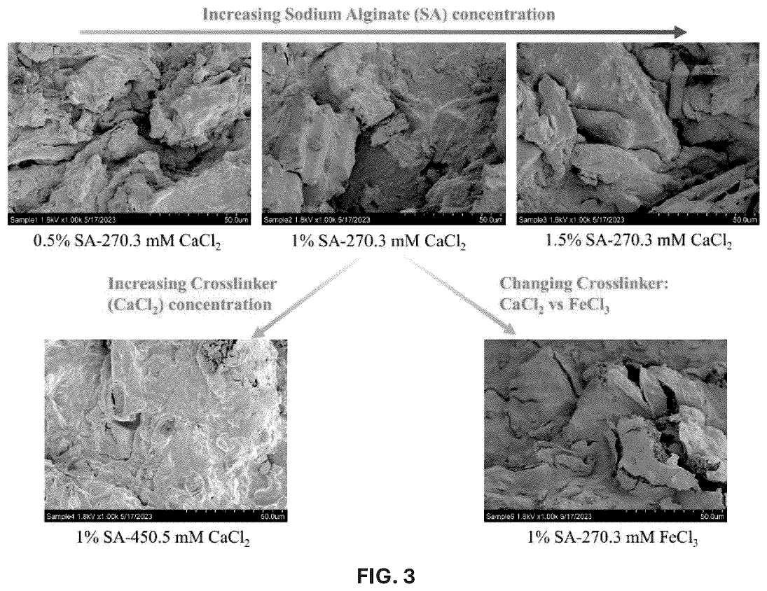

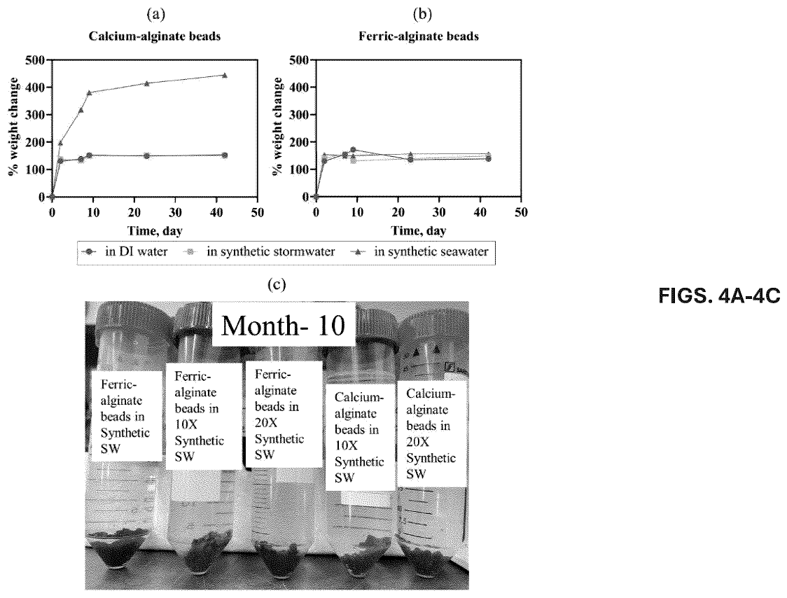

SEM Microscopy to characterize bead surface: The surface morphology of the dried composite alginate beads was assessed using scanning electron microscopy for visual characterization (images are presented at equal magnification; scale bar=50 μm) (FIG. 3). All types of beads have rough and porous surface structures containing multiple cracks and pores (many >10 μm). When the sodium alginate concentration was increased in the calcium alginate bead recipe (0.5 to 1.5% sodium alginate), thicker and smoother alginate gel structures formed. (68) Similar effects were observed when the crosslinker concentration was increased (3% to 5% CaCl₂)). When the crosslinker cation composition was changed from CaCl₂) to FeCl₃, a denser alginate coating was noted with many large cracks and pores. Because the trivalent Fe³⁺ binds to both polyguluronate (GG) and polymannuronate (GM) groups of alginates in a porous three-dimensional structure, ferric alginate beads form stronger bonds than calcium alginate beads (calcium binds to the GG groups only). (52) As a result, Fe³⁺ beads have higher mechanical strength and higher porosity (FIG. 2) and swell less than Ca²⁺ beads (FIG. 4). Similarly rough and porous surfaces of composite alginate beads are also reported in previous studies. (69) Additional SEM images can be found in the supplementary information section (FIG. S13).

Bead swelling: Calcium alginate beads swelled more than ferric alginate beads when in the presence of a high ionic strength solution (FIG. 4). It was observed that calcium alginate beads did not swell differently in DI water and in synthetic stormwater (p>0.9999); however, the Ca²⁺ beads swelled ˜3 times more in synthetic seawater than in synthetic stormwater (p=0.0372). Conversely, Fe³⁺ alginate beads exhibited similar swelling under the three ionic strength conditions tested (p=0.9684). There were no significant differences when comparing swelling of Ca²⁺ alginate beads and Fe³⁺ alginate beads in DI water (p=0.9742) and in synthetic stormwater (p=0.9651); however, when the solution ionic strength increased to the level of synthetic seawater, the Ca²⁺ alginate beads swelled approximately 3 times more than Fe³⁺ alginate beads (p=0.0476). Synthetic seawater had 140 times greater ionic strength than synthetic stormwater (0.7M vs 0.005M). The trivalent Fe³⁺ beads have a complex three-dimensional Fe³⁺-alginate structure, whereas the divalent Ca²⁺ beads have two-dimensional Ca²⁺-alginate structure. (52) Analogous trends in swelling results have also been reported for trivalent Al³⁺ alginate beads compared with divalent Ca²⁺ alginate beads. (70) Because both Ca²⁺ beads and Fe³⁺ beads swelled approximately the same amount in synthetic stormwater (p=0.9651; the relative difference in swelling ratios were <2% after 42 days), both types of beads will also exhibit similar swelling in most relevant GSI applications. For applications where the beads will be exposed to high ionic strength conditions such as bioremediation/bioaugmentation of sediments in estuaries, Fe³⁺ beads can be selected.

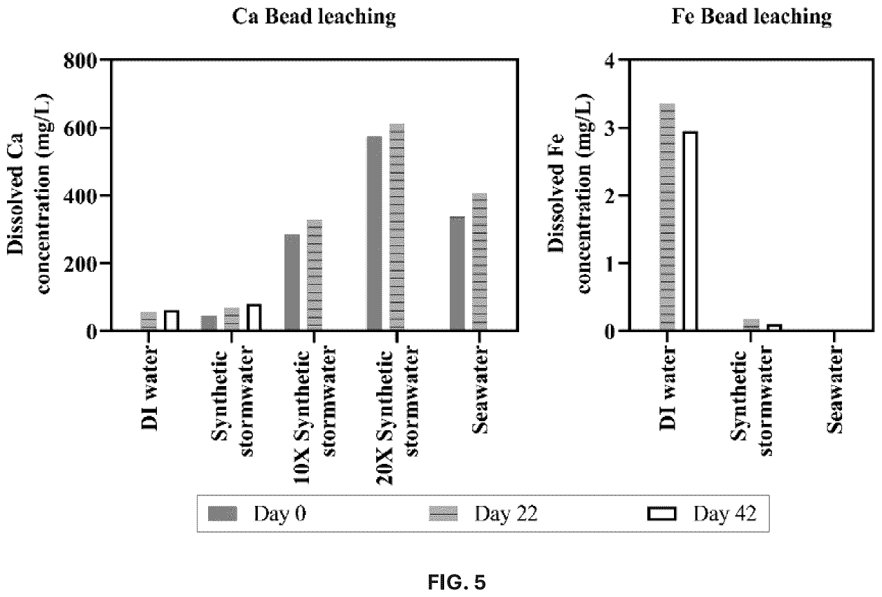

Bead leaching: Calcium alginate beads were maintained in DI water, synthetic stormwater, and synthetic seawater to quantify calcium release in the dissolved phase. When Ca²⁺ alginate beads were maintained in DI water and in high ionic strength solution (synthetic seawater), the relative increase in the dissolved calcium concentration increase was higher in DI water (˜78-fold higher after 22 days; initial Ca concentration=0.73 mg/L; Ca concentration after 22 days=57.5 mg/L) than in synthetic seawater (˜21% increment after 22 days; initial Ca concentration=337.21 416 mg/L; Ca concentration after 22 days=408.4 mg/L) (FIG. 5 and Table S2). Swelling and leaching behaviors of alginate beads are known to be impacted by the osmotic pressure from the solution where the beads are maintained. (71,72) The change in osmotic pressure with the increase in solution ionic strength may retard some of the cationic crosslinker leaching into the solution. Likely as a result of this phenomenon, we observed a higher percent Ca²⁺ release in DI water than in solutions with higher ionic strengths. Crosslinker leaching may also be impacted by ion exchange potential. (70) Because calcium was not leached relatively more under the increased ionic strength solution conditions, these beads are likely stable in the presence of a higher ionic strength solution under environmentally relevant conditions for stormwater systems, such as typical freshwater road up to salt induced deicing influxes. To ensure the beads remained stable, beads were maintained in synthetic stormwater, 10× synthetic stormwater, 20× synthetic stormwater, and synthetic seawater for 10 months; the beads did not disintegrate (FIG. 4(c), FIG. S10).

TABLE S 2
Dissolved Ca and dissolved Fe concentrations in DI water, synthetic stormwater,
10X synthetic stormwater, 20X synthetic stormwater, and synthetic seawater
(values that were not measured are shown with hyphen).

| | | | SW | 10X SW | 20X SW | Seawater | |
|---|---|---|---|---|---|---|---|
| | | | (Ionic | (Ionic | (Ionic | (Ionic | |
| | | DI | strength = | strength = | strength = | strength = | |
| | Day | water | 0.005M) | 0.05M) | 0.1M) | 0.7M) | Note |
| Dissolved | 0 | 0.728 | 47.1276 | 285.9766 | 572.7462 | 337.2148 | Ca beads |
| Ca | 22 | 57.525 | 69.5994 | 327.925 | 610.4254 | 408.356 | were kept in |
| (mg/L) | 42 | 62.7484 | 79.6198 | — | — | — | the solvents |
| Dissolved | 0 | 0.0026 | <0.0000 | — | — | <0.0000 | Fe beads |
| Fe | 22 | 3.3592 | 0.1768 | — | — | <0.0000 | were kept in |
| (mg/L) | 42 | 2.9484 | 0.1014 | — | — | — | the solvents |

Iron release from Fe³⁺ alginate beads was lower than calcium release from Ca²⁺ alginate beads (e.g., after 42 days, dissolved iron concentration in synthetic stormwater was ˜20 times lower than dissolved calcium concentration in synthetic stormwater) (FIG. 5). It was hypothesized that the stronger bonds between trivalent iron and alginate improve bead durability (i.e., less swelling/leaching). Dissolved total iron concentrations in DI water were 0.003 mg/L on day 0, 3.36 mg/L on day 22 (increased 1120-fold), and 2.95 mg/L on day 42 (increased 983-fold). In synthetic stormwater, iron concentrations were below the detection limit on day 0, 0.17 mg/L on day 22, and 0.10 mg/L on day 42. Dissolved iron concentrations were under the detection limit for synthetic seawater on day 0 and day 22 (synthetic seawater data could not be collected on day 42). Iron beads will thus not likely leach concerning quantities of iron crosslinkers in environmentally relevant high ionic strength stormwater runoff (i.e., dissolved iron concentrations were even lower than dissolved calcium concentrations in both synthetic stormwater and synthetic seawater). The dissolved iron values measured, however, did not likely constitute all the iron leached from the beads. Specifically, visible brown iron precipitates were present in the synthetic seawater, whereas no precipitates were visible in DI water (FIG. S10). Ferric beads in DI water yielded a solution pH of ˜3.5, whereas the pH was ˜6.15 in synthetic seawater (stored in sealed containers; no change in solution pH over the course of 5 months). Iron solubility is strongly associated with the pH and the redox potential of the solution. (73) As the solution container was sealed and there was de minimis headspace, the redox potential of the synthetic seawater is unlikely to change due to oxidation due to air presence in the vial (redox potential of seawater varies between 0.7 and 0.8V74). When pH is below 5, dissolved iron can exist as Fe³⁺, FeOH⁺⁺, and Fe(OH)²⁺, whereas stable dark brown colloidal suspensions of ferric hydroxides precipitate in the pH range of 5 to 8 at equilibrium. (73) Because Ca²⁺ alginate beads and Fe³⁺ alginate beads do not disintegrate even under high ionic strength solution conditions, both the beads could be applied in environmentally relevant field bioretention cells to treat stormwater runoff.

### Bioaugmentation Potentials of BioSorp Beads.

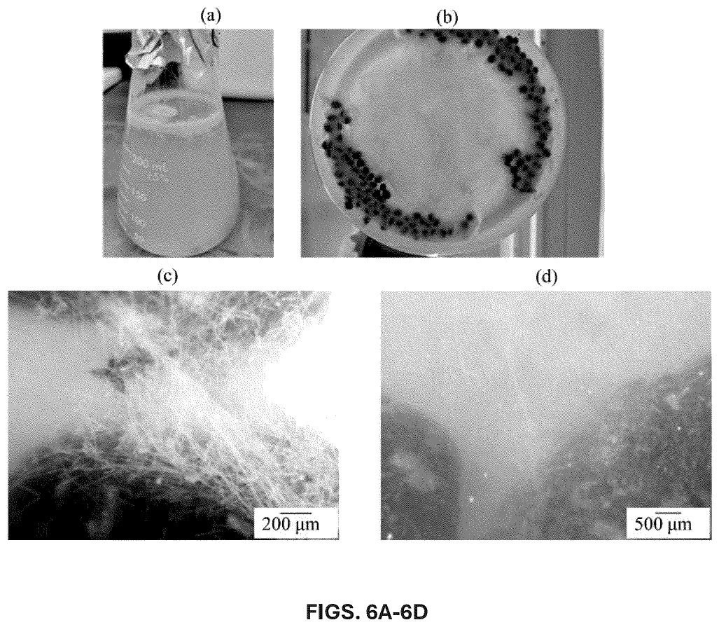

Viability of encapsulated fungi: White rot fungi (WRF) were able to remain viable in the BioSorp Beads and could grow from the beads following an extended dried storage period, which is desired for practical bioaugmentation efforts. The inventors successfully encapsulated Trametes versicolor fungi in the beads along with PAC, FeWTR, wood flour, and AQDS by entrapping fungi in either Ca²⁺ alginate or Fe³⁺ alginate crosslinkers. When added to growth media (malt extract media and modified Kirk's liquid culture media (75) in this case), dried BioSorp Beads were capable of growing fungal fruiting bodies on the surface of the beads (FIG. 6), thereby demonstrating viability of the encapsulated organisms (i.e., no microbial growth occurred in beads that did not contain any fungal culture). It was also observed that WRF diffused out of the dried BioSorp Beads into the culture media through the cracks and pores, demonstrating potential for bio-augmenting the bioretention cells. Encapsulation is known to increase the shelf life of microorganisms. (76) The inventors maintained the air-dried beads in sealed tubes at room temperature for three months and observed similar fungal growth from stored beads and freshly prepared beads. This extended storage scenario has also been observed for alginate coated entomopathogenic fungus (Metarhizium anisopliae), where the authors reported fungal growth even after ten months. (77) Fungal viability can reportedly be substantially increased by storing beads refrigerated (at ˜4-5 deg C.). (47)

Similar fungal growth was observed for both air-dried and oven-dried beads (OD600 was 1.5 after two weeks of bead inoculation into malt extract media). Encapsulation is known to protect microorganisms by providing a higher resistance to the environment. (78) This was evident in case of the oven-dried beads. Even though the oven-dried beads were dried at 70° Celsius for 8 hours, no visible decline in fungal growth was observed compared to the air-dried beads. Encapsulated microorganisms are known to exhibit a decreased tendency to wash out and have higher viability, stability, and activity. (79) Encapsulated microorganisms also reportedly produce more extracellular enzymes that can degrade contaminants outside of the cells. (79) Multiple organic and inorganic polymer matrices could potentially be applied to accomplish this encapsulation process; however, all matrices do not have the same beneficial impacts on microorganisms. Inorganic polymer matrices, such as sol-gels, hold a higher resistance toward microbial and chemical attack and are more durable, but natural organic polymers are non-toxic, biodegradable, and affordable. (80,81) Chitosan is another natural organic polysaccharide (like alginate) that could alternatively be used to encapsulate microorganisms for water treatment. (82) Chitosan powder is often mixed with alginate to increase the mechanical strength of the hybrid gel beads. (83-85)

Creating a beneficial micro-environment to enhance viability and biodegradation: The inventors incorporated wood flour into the BioSorp Beads with the goal of providing a maintenance substrate (nutrient supplement) for the fungi to promote longevity when deployed in the field. Loomis et al. (47) reported that Phanerochaete chrysosporium encapsulated alginate beads (stored at room temperature) exhibited high viability even after nine months when an external nutrient supplement (i.e., wood flour or corncob grits) was present in the bead structure, whereas drastic depletion in viability occurred after only two months in beads that did not contain any nutrient supplementation. This type of maintenance substrate application is not exclusive to WRF and wood flour. For example, shredded straw, (86) grass, (86) newspaper, (87) cotton, (87) rice husk, (88) wood flour, (89) and chlorella (90) have previously been used as external/supplemental carbon sources to increase denitrification in synthetic wastewater and domestic sewage. Maintenance substrates are also reported to increase pollutant removal efficiencies in stormwater bioretention systems. For example, woodchips act as carbon sources and electron donors in biological denitrifying woodchip bioreactors. (91-94) Newspaper (as carbon source and electron donor) has also been added in bioretention cells to improve the stormwater denitrification performance. (95,96) BioSorp Beads could also be adaptively prepared by encapsulating alternative carbon sources and contaminant degrading microbes and used for stormwater and/or wastewater treatment applications.

The acidic environment created by ferric alginate beads may specifically promote fungal growth in bioretention cells. Indeed, Johannes et al. (97) demonstrated that bacterial and fungal growth in soil were dramatically impacted by variations of pH, where fungal growth increased with decreasing pH (optimum at ˜pH 4-5) and bacterial growth increased with increasing pH (optimum at ˜pH 7-8). Because Fe³⁺ alginate beads generate lower pH conditions (˜3.5) than Ca²⁺alginate beads (˜6.2), Fe³⁺ alginate beads could improve fungal deployment in bioretention cells by creating favorable conditions wherein fungi can more successfully outcompete bacterial biofilms. Fe³⁺ alginate beads could also be suitable for bioaugmenting acidophilic heterotrophic bacteria capable of degrading various aromatic hydrocarbons. (98) Fe³⁺ alginate beads could also potentially facilitate PFAS sorption and biodegradation in bioretention systems (in the presence of adequate NH₄⁺ ions) by providing the acidic environment (optimal around pH 4) and Fe³⁺ ions needed by the PFAS-degrading acidophilic Feammox bacteria (i.e., Acidimicrobium sp. strain A6). (99) Conversely, calcium alginate beads may be more suitable for bioaugmenting neutrophilic microbes (most bacteria are neutrophiles (100)).

The inventors incorporated AQDS (as external electron shuttles (EES)) in the beads with the goal of enhancing fungal viability potential and biodegradation rates in GSI system. Electron shuttles are capable of reversible oxidation and reduction reactions and can aid biological transformations by acting as redox mediators. (101) Electron shuttles are known to also enhance degradation of some recalcitrant organic contaminants by either donating/accepting electrons for microbial metabolism or donating/accepting electrons to microorganisms for contaminant transformation. (102) Previous studies have reported increased biodegradation of a variety of contaminants in the presence of AQDS. For example, Aulenta et al. (103) used AQDS as EES to facilitate microbial dechlorination of trichloroethene (TCE) (102) and Zhou et al. (104) demonstrated that AQDS, encapsulated in chitosan gel beads, can provide efficient and environmentally friendly anaerobic decolorization of azo dyes. (103) Thus, the addition of AQDS in BioSorp Beads can enhance trace organic biodegradation in bioretention cells. Furthermore, AQDS can also facilitate denitrification in GSI in the presence of ferric sludge (FeWTR). Denitrifying and iron reducing bacteria, present in saturated anaerobic bioretention zone, can use AQDS and iron oxide rich FeWTR to stimulate dissolved N removal. (43,59,105-107) PAC itself can also act as electron shuttle by accepting and/or donating electrons because of the condensed conjugated pi-electrons present in the structure. (102, 108) Fungal viability in bioretention cell may further improve because PAC can act as growth surface for the biofilms. (109) BioSorp Beads as a platform technology.

The work presented herein is a proof-of-concept for enabling targeted microorganism deployments, i.e., bioaugmentation specifically designed for GSI applications; however, the BioSorp Beads are fundamentally platform technology. BioSorp Beads are able to provide favorable micro-environments for microbial growth and bioaugmentation. The beads are made of non-toxic materials (i.e., alginate), are physically durable, and incorporate valorized/recycled waste products (i.e., ferric sludge, wood flour). Because the production process is economical and scalable, the beads can be practically applied and adapted for field bioaugmentation. Although it was shown here with encapsulated white rot fungi as the model bioaugmentation organism for the design application goal in green stormwater infrastructure, the BioSorp Bead can easily be modified to encapsulate other types of fungal or bacterial species for specific needs. For example, BioSorp Beads containing denitrifying bacteria could be incorporated in the bottom anaerobic saturated zones found in some bioretention designs, (110-112) while beads containing nitrifying bacteria could be added to the upper aerobic unsaturated bioretention zones to maximize nitrogen nutrient removal from stormwater runoff. The sorptive materials used in the beads can also be potentially interchanged with other types of sorptive materials, such as powdered biochar, (18) aluminum-based water treatment residuals, (113) iron oxide coated sorbents, (114) zinc oxide coated sorbents, (115) manganese oxide coated sand, (116, 117) etc. to specifically tune capture different contaminants. Because the design goal for the beads was stormwater treatment, it was decided to use iron-based sorptive materials because iron materials have previously been applied for dissolved P removal in stormwater (118, 119) and because Al- or Zn-based sorbents hold the risks of spreading toxic Al or Zn metals (120, 121) in the environment.

BioSorp Beads are a platform technology able to encapsulate and deploy multiple types of microorganisms or nutrients for a variety of stormwater, wastewater, bioremediation, or other biotechnology applications. The bead physical properties can also be tuned according to specific needs by adjusting alginate and/or crosslinker concentrations and crosslinker types. One example potential application of BioSorp Beads could be incorporation into fluidized bed reactors (FBR), a highly effective technology to treat high strength wastewater. (122, 123) Some key challenges while operating FBR are preventing microbial washout, retaining active biomass, and maintaining minimum substrate concentrations needed to sustain microbial redox reactions. Preventing microbial washout is important for slow growing microorganisms, such as anammox bacteria (capable of removing high strength ammonia from wastewater). (88) BioSorp Beads can address this problem by working as microbial seeds and/or reservoirs by retaining the encapsulated microorganisms in FBR system. Biodegradation of trace organic contaminants is often difficult in wastewater that contains complex mixtures due to issues with minimum substrate concentrations under variable conditions. (124) Transformation of TOrCs frequently requires proximity to the biodegrading microorganisms for extended time periods before key enzymes are upregulated. (125, 126) The black carbon present in the BioSorp Beads could sorb TOrCs and retain the encapsulated microorganisms in close proximity to enhance biodegradation potential. Another possible application of adapted BioSorp Beads could be in bioremediation of hazardous materials. (127) For instance, PCB degrading microorganisms (128) could be encapsulated in BioSorp Beads to biodegrade PCB in river/estuary sediments. The presence of iron water treatment residuals, PAC, and wood flour in the beads increases the bead weight and density, which would be needed to keep the deployed beads in close proximity to the contaminated river/lake sediment and facilitate contaminant removal. Along with contaminant degrading microorganisms, limiting micronutrients/vitamins for biodegradation of many hazardous materials could also be incorporated into the beads. For example, encapsulated vitamin B12 and PCB degrading microorganisms could potentially enhance and sustain both chemical and microbial reductive dechlorination of PCBs in contaminated sediments. (129) The scalable production process and the tunable characteristics make BioSorp Bead an adaptable platform for a spectrum of remediation and biotechnology applications.

### Conclusions

Provided herein is the development of a cost-effective method to enable rapid capture and subsequent sustained biodegradation of dissolved contaminants while delivering contaminant-degrading microorganisms (i.e., bioaugmentation) in green stormwater infrastructure. The inventors developed a novel composite alginate bead media (i.e., ‘BioSorp Bead’) containing PAC, WRF, wood flour, AQDS, and iron-based water treatment residuals, and demonstrated the bead's potential toward the application design goal of bioaugmenting GSI. This mechanically robust novel assemblage of materials holds promise to enable bioaugmentation and sustained contaminant degradation in GSI, including bioretention cells, without compromising hydraulic conductivity needed for rapid infiltration. Bead properties, such as surface area, pore volume, mechanical strength, etc. are customizable and vary with respect to the changes in the recipes. It was also demonstrated that white rot fungi remain viable in a protected microenvironment created by the encapsulated conditions and thus BioSorp Beads have the practical potential to bioaugment GSI and provide superior stormwater treatment than traditional management systems.

Urban stormwater is well-known to contain a diverse suite of contaminants that have deleterious impacts on the environment. 1 The short hydraulic residence time and low sorption potential associated with most high-hydraulic conductivity, sand-based porous media in GSI perform poorly at removing many dissolved phase contaminants, including polar trace organics and dissolved nutrients, thereby generating concomitant risk for groundwater contamination. (3, 129) Indeed, amended bioretention media are capable of capturing increased quantities of trace organic contaminants. (130) BioSorp Beads could be incorporated into new bioretention systems or amended post-construction into existing GSI systems for bioaugmentation. BioSorp Beads are designed to directly address the wicked problem with hydrophilic compounds by: (1) rapidly capturing contaminants onto the black carbon materials via chemical sorption during the rapid infiltration stages and (2) encapsulating and bioaugmenting contaminant degrading microorganisms in GSI systems. It is posited that synergizing rapid chemical capture during storm events with subsequent, slower biodegradation during inter-storm periods can optimize GSI system performance by maximizing stormwater volume infiltrated and contaminant mass removed. Decoupling the hydraulic residence time from contaminant residence time using BioSorp Beads can elevate GSI performance for hydrophilic organic contaminants, in much the same way activated sludge revolutionized wastewater treatment by decoupling hydraulic residence time from solids retention time.

### BIBLIOGRAPHY

1 L. Mutzner, K. Zhang, R. G. Luthy, H. P. H. Arp and S. Spahr, Urban stormwater capture for water supply: look out for persistent, mobile and toxic substances, Environ. Sci.: Water Res. Technol., 2023, 9, 3094-3102.
2 Z. Kong, Y. Song, Z. Shao and H. Chai, Biochar-pyrite bi-layer bioretention system for dissolved nutrient treatment and by-product generation control under various stormwater conditions, Water Res, 2021, 206, 117737.
3 T. F. M. Rodgers, L. Wu, X. Gu, S. Spraakman, E. Passeport and M. L. Diamond, Stormwater Bioretention Cells Are Not an Effective Treatment for Persistent and Mobile Organic Compounds (PMOCs), Environ Sci Technol, 2022, 56, 6349-6359.
4 M. E. Dietz, Low Impact Development Practices: A Review of Current Research and Recommendations for Future Directions, Water Air Soil Pollut, 2007, 186, 351-363.
5 S. Alam, A. Borthakur, S. Ravi, M. Gebremichael and S. K. Mohanty, Managed aquifer recharge implementation criteria to achieve water sustainability, Science of The Total Environment, 2021, 768, 144992.
6 P. Jokela and E. Kallio, Sprinkling and Well Infiltration in Managed Aquifer Recharge for Drinking Water Quality Improvement in Finland, J Hydrol Eng, DOI: 10.1061/(ASCE) HE. 1943-5584.0000975.
7 C. Yoo, J. M. Ku, C. Jun and J. H. Zhu, Simulation of infiltration facilities using the SEEP/W model and quantification of flood runoff reduction effect by the decrease in CN, Water Science and Technology, 2016, 74, 118-129.
8 G. H. LeFevre, M. D. Hendricks, M. E. Carrasquillo, L. E. McPhillips, B. K. Winfrey and J. R. Mihelcic, The Greatest Opportunity for Green Stormwater Infrastructure Is to Advance Environmental Justice, Environ Sci Technol, 2023, 57, 19088-19093.
9 A. Dietrich, R. Yarlagadda and C. Gruden, Estimating the potential benefits of green stormwater infrastructure on developed sites using hydrologic model simulation, Environ Prog Sustain Energy, 2017, 36, 557-564.
10 J. Liu, D. J. Sample, C. Bell and Y. Guan, Review and Research Needs of Bioretention Used for the Treatment of Urban Stormwater, Water (Basel), 2014, 6, 1069-1099.
11 C. M. Rodak, T. L. Moore, R. David, A. D. Jayakaran and J. R. Vogel, Urban stormwater characterization, control, and treatment, Water Environment Research, 2019, 91, 1034-1060.
12 C. Hsieh and A. P. Davis, Evaluation and optimization of bioretention media for treatment of urban storm water runoff, Journal of Environmental Engineering, 2005, 131, 1521-1531.
13 J. R. Masoner, D. W. Kolpin, I. M. Cozzarelli, L. B. Barber, D. S. Burden, W. T. Foreman, K. J. Forshay, E. T. Furlong, J. F. Groves, M. L. Hladik, M. E. Hopton, J. B. Jaeschke, S. H. Keefe, D. P. Krabbenhoft, R. Lowrance, K. M. Romanok, D. L. Rus, W. R. Selbig, B. H. Williams and P. M. Bradley, Urban Stormwater: An Overlooked Pathway of Extensive Mixed Contaminants to Surface and Groundwaters in the United States, Environ Sci Technol, 2019, 53, 10070-10081.
14 S. M. Elliott, R. L. Kiesling, A. M. Berg and H. L. Schoenfuss, A pilot study to assess the influence of infiltrated stormwater on groundwater: Hydrology and trace organic contaminants, Water Environment Research, DOI: 10.1002/wer.10690.
15 A. Behbahani, R. J. Ryan and E. R. Mckenzie, Long-term simulation of potentially toxic elements (PTEs) accumulation and breakthrough in infiltration-based stormwater management practices (SMPs), J Contam Hydrol, DOI: 10.1016/j.jconhyd.2020.103685.
16 A. Zubair, A. Hussain, M. A. Farooq and H. N. Abbasi, Impact of storm water on groundwater quality below retention/detention basins, Environ Monit Assess, 2010, 162, 427-437. K. Björklund and L. Li, Removal of organic contaminants in bioretention medium amended with activated carbon from sewage sludge, Environmental Science and Pollution Research, 2017, 24, 19167-19180.
18 B. A. Ulrich, E. A. Im, D. Werner and C. P. Higgins, Biochar and Activated Carbon for Enhanced Trace Organic Contaminant Retention in Stormwater Infiltration Systems, Environ Sci Technol, 2015, 49, 6222-6230.
19 M. Hassan, Y. Liu, R. Naidu, S. J. Parikh, J. Du, F. Qi and I. R. Willett, Influences of feedstock sources and pyrolysis temperature on the properties of biochar and functionality as adsorbents: A meta-analysis, Science of The Total Environment, 2020, 744, 140714.
20 B. A. Ulrich, M. Loehnert and C. P. Higgins, Improved contaminant removal in vegetated stormwater biofilters amended with biochar, Environ. Sci.: Water Res. Technol., 2017, 3, 726-734.
21 I. LeviRam, A. Gross, A. Lintern, R. Henry, C. Schang, M. Herzberg and D. McCarthy, Sustainable micropollutant bioremediation via stormwater biofiltration system, Water Res, 2022, 214, 118188.
22 Z. Kong, Y. Song, M. Xu, Y. Yang, X. Wang, H. Ma, Y. Zhi, Z. Shao, L. Chen, Y. Yuan, F. Liu, Y. Xu, Q. Ni, S. Hu and H. Chai, Multi-media interaction improves the efficiency and stability of the bioretention system for stormwater runoff treatment, Water Res, 2024, 250, 121017.
23 V. K. Vaithyanathan, O. Savary and H. Cabana, Performance evaluation of biocatalytic and biostimulation approaches for the remediation of trace organic contaminants in municipal biosolids, Waste Management, 2021, 120, 373-381.
24 A. C. Portmann, G. H. LeFevre, R. Hankawa, D. Werner and C. P. Higgins, The regenerative role of biofilm in the removal of pesticides from stormwater in biochar-amended biofilters, Environ. Sci.: Water Res. Technol., 2022, 8, 1092-1110.
25 X. Min, W. Li, Z. Wei, R. Spinney, D. D. Dionysiou, Y. Seo, C.-J. Tang, Q. Li and R. Xiao, Sorption and biodegradation of pharmaceuticals in aerobic activated sludge system: A combined experimental and theoretical mechanistic study, Chemical Engineering Journal, 2018, 342, 211-219.
26 C. P. Muerdter, C. K. Wong and G. H. LeFevre, Emerging investigator series: the role of vegetation in bioretention for stormwater treatment in the built environment: pollutant removal, hydrologic function, and ancillary benefits, Environ. Sci.: Water Res. Technol., 2018, 4, 592-612.
27 G. H. LeFevre, R. M. Hozalski and P. J. Novak, The role of biodegradation in limiting the accumulation of petroleum hydrocarbons in raingarden soils, Water Res, 2012, 46, 6753-6762.
28 G. H. LeFevre, P. J. Novak and R. M. Hozalski, Fate of Naphthalene in Laboratory-Scale Bioretention Cells: Implications for Sustainable Stormwater Management, Environ Sci Technol, 2012, 46, 995-1002.
29 M. B. Asif, F. I. Hai, L. Singh, W. E. Price and L. D. Nghiem, Degradation of Pharmaceuticals and Personal Care Products by White-Rot Fungi—a Critical Review, Curr Pollut Rep, 2017, 3, 88-103.
30 S. Wang, W. Li, L. Liu, H. Qi and H. You, Biodegradation of decabromodiphenyl ethane (DBDPE) by white-rot fungus Pleurotus ostreatus: Characteristics, mechanisms, and toxicological response, J Hazard Mater, 2022, 424, 127716.
31 E. A. Wiener and G. H. LeFevre, White Rot Fungi Produce Novel Tire Wear Compound Metabolites and Reveal Underappreciated Amino Acid Conjugation Pathways, Environ Sci Technol Lett, 2022, 9, 391-399.
32 C. Rauert, N. Charlton, E. D. Okoffo, R. S. Stanton, A. R. Agua, M. C. Pirrung and K. V Thomas, Concentrations of Tire Additive Chemicals and Tire Road Wear Particles in an Australian Urban Tributary, Environ Sci Technol, 2022, 56, 2421-2431.
33 M. Brinkmann, D. Montgomery, S. Selinger, J. G. P. Miller, E. Stock, A. J. Alcaraz, J. K. Challis, L. Weber, D. Janz, M. Hecker and S. Wiseman, Acute Toxicity of the Tire Rubber-Derived Chemical 6PPD-quinone to Four Fishes of Commercial, Cultural, and Ecological Importance, Environ Sci Technol Lett, 2022, 9, 333-338.
34 J. K. McIntyre, J. Prat, J. Cameron, J. Wetzel, E. Mudrock, K. T. Peter, Z. Tian, C. Mackenzie, J. Lundin, J. D. Stark, K. King, J. W. Davis, E. P. Kolodziej and N. L. Scholz, Treading Water: Tire Wear Particle Leachate Recreates an Urban Runoff Mortality Syndrome in Coho but Not Chum Salmon, Environ Sci Technol, 2021, 55, 11767-11774.
35 Z. Tian, H. Zhao, K. T. Peter, M. Gonzalez, J. Wetzel, C. Wu, X. Hu, J. Prat, E. Mudrock, R. Hettinger, A. E. Cortina, R. G. Biswas, F. V. C. Kock, R. Soong, A. Jenne, B. Du, F. Hou, H. He, R. Lundeen, A. Gilbreath, R. Sutton, N. L. Scholz, J. W. Davis, M. C. Dodd, A. Simpson, J. K. McIntyre and E. P. Kolodziej, A ubiquitous tire rubber-derived chemical induces acute mortality in coho salmon, Science (1979), 2021, 371, 185-189.
36 J. M. Wolfand, G. H. LeFevre and R. G. Luthy, Metabolization and degradation kinetics of the urban-use pesticide fipronil by white rot fungus Trametes versicolor, Environ. Sci.: Processes Impacts, 2016, 18, 1256-1265.
37 S. Mukherjee, B. Sarkar, V. K. Aralappanavar, R. Mukhopadhyay, B. B. Basak, P. Srivastava, O. Marchut-Mikołajczyk, A. Bhatnagar, K. T. Semple and N. Bolan, Biochar-microorganism interactions for organic pollutant remediation: Challenges and perspectives, Environmental Pollution, 2022, 308, 119609.
38 D. Pandey, A. Daverey and K. Arunachalam, Biochar: Production, properties and emerging role as a support for enzyme immobilization, J Clean Prod, 2020, 255, 120267.
39 E. Taskin, M. T. Brana, C. Altomare and E. Loffredo, Biochar and hydrochar from waste biomass promote the growth and enzyme activity of soil-resident ligninolytic fungi, Heliyon, 2019, 5, e02051.
40 C. J. Mitchell, A. D. Jayakaran and J. K. McIntyre, Biochar and fungi as bioretention amendments for bacteria and PAH removal from stormwater, J Environ Manage, 2023, 327, 116915.
41 A. Taylor, J. Wetzel, E. Mudrock, K. King, J. Cameron, J. Davis and J. McIntyre, Engineering Analysis of Plant and Fungal Contributions to Bioretention Performance, Water, 2018, 10, 1226.
42 D. Mohapatra, S. K. Rath and P. K. Mohapatra, in Mycoremediation and Environmental Sustainability: Volume 2, ed. R. Prasad, Springer International Publishing, Cham, 2018, pp. 181-212.
43 R. Sanchis, A. Dejoz, I. Vázquez, E. Vilarrasa-García, J. Jiménez-Jiménez, E. Rodríguez-Castellón, J. M. López Nieto and B. Solsona, Ferric sludge derived from the process of water purification as an efficient catalyst and/or support for the removal of volatile organic compounds, Chemosphere, 2019, 219, 286-295.
44 H. Smaili and C. Ng, Adsorption as a remediation technology for short-chain per- and polyfluoroalkyl substances (PFAS) from water—a critical review, Environ. Sci.: Water Res. Technol., 2023, 9, 344-362.
45 H. Lin, Y. Wang, J. Niu, Z. Yue and Q. Huang, Efficient Sorption and Removal of Perfluoroalkyl Acids (PFAAs) from Aqueous Solution by Metal Hydroxides Generated in Situ by Electrocoagulation, Environ Sci Technol, 2015, 49, 10562-10569.
46 M. K. Gibbons and G. A. Gagnon, Understanding removal of phosphate or arsenate onto water treatment residual solids, J Hazard Mater, 2011, 186, 1916-1923.
47 A. K. Loomis, A. M. Childress, D. Daigle and J. W. Bennett, Alginate Encapsulation of the White Rot Fungus Phanerochaete chrysosporium, Curr Microbiol, 1997, 34, 127-130.
48 Z. Chen, Y. Wang, X. Jiang, D. Fu, D. Xia, H. Wang, G. Dong and Q. Li, Dual roles of AQDS as electron shuttles for microbes and dissolved organic matter involved in arsenic and iron mobilization in the arsenic-rich sediment, Science of The Total Environment, 2017, 574, 1684-1694.
49 L. E. de-Bashan and Y. Bashan, Immobilized microalgae for removing pollutants: Review of practical aspects, Bioresour Technol, 2010, 101, 1611-1627.
50 V. Rocher, J.-M. Siaugue, V. Cabuil and A. Bee, Removal of organic dyes by magnetic alginate beads, Water Res, 2008, 42, 1290-1298.
51 B. Wang, Y. Wan, Y. Zheng, X. Lee, T. Liu, Z. Yu, J. Huang, Y. S. Ok, J. Chen and B. Gao, Alginate-based composites for environmental applications: a critical review, Crit Rev Environ Sci Technol, 2019, 49, 318-356.
52 D. Massana Roquero, A. Othman, A. Melman and E. Katz, Iron (III)-cross-linked alginate hydrogels: a critical review, Mater Adv, 2022, 3, 1849-1873.
53 M. Naderi, Progress in Filtration and Separation; Chapter Fourteen-Surface Area: Brunauer-Emmett-Teller (BET), Academic Press, 2015, 585-608, ISBN 9780123847461, https://doi.org/10.1016/B978-0-12-384746-1.00014-8.
54 C. P. Kaluarachchi, V. W. Or, Y. Lan, C. K. Madawala, E. S. Hasenecz, D. R. Crocker, C. K. Morris, H. D. Lee, K. J. Mayer, J. S. Sauer, C. Lee, G. Dorce, F. Malfatti, E. A. Stone, C. D. Cappa, V. H. Grassian, K. A. Prather and A. V Tivanski, Size-Dependent Morphology, Composition, Phase State, and Water Uptake of Nascent Submicrometer Sea Spray Aerosols during a Phytoplankton Bloom, ACS Earth Space Chem, 2022, 6, 116-130.
55 C. P. Kaluarachchi, V. W. Or, Y. Lan, E. S. Hasenecz, D. Kim, C. K. Madawala, G. P. Dorcé, K. J. Mayer, J. S. Sauer, C. Lee, C. D. Cappa, T. H. Bertram, E. A. Stone, K. A. Prather, V. H. Grassian and A. V Tivanski, Effects of Atmospheric Aging Processes on Nascent Sea Spray Aerosol Physicochemical Properties, ACS Earth Space Chem, 2022, 6, 2732-2744.
56 C. K. Madawala, H. D. Lee, C. P. Kaluarachchi and A. V Tivanski, Probing the Water Uptake and Phase State of Individual Sucrose Nanoparticles Using Atomic Force Microscopy, ACS Earth Space Chem, 2021, 5, 2612-2620.
57 P. Nandanwar, R. Jugade, V. Gomase, A. Shekhawat, A. Bambal, D. Saravanan and S. Pandey, Chitosan-Biopolymer-Entrapped Activated Charcoal for Adsorption of Reactive Orange Dye from Aqueous Phase and CO2 from Gaseous Phase, Journal of Composites Science, 2023, 7, 103.
58 S. Peretz, D. F. Anghel, E. Vasilescu, M. Florea-Spiroiu, C. Stoian and G. Zgherea, Synthesis, characterization and adsorption properties of alginate porous beads, Polymer Bulletin, 2015, 72, 3169-3182.
59 F. J. Cervantes, C. H. Gutiérrez, K. Y. López, M. I. Estrada-Alvarado, E. R. Meza-Escalante, A.-C. Texier, F. Cuervo and J. Gómez, Contribution of quinone-reducing microorganisms to the anaerobic biodegradation of organic compounds under different redox conditions, Biodegradation, 2008, 19, 235-246.
60 J. A. Field, Recalcitrance as a catalyst for new developments, Water Science and Technology, 2001, 44, 33-40.
61 A. B. dos Santos, F. J. Cervantes and J. B. van Lier, Azo dye reduction by thermophilic anaerobic granular sludge, and the impact of the redox mediator anthraquinone-2,6-disulfonate (AQDS) on the reductive biochemical transformation, Appl Microbiol Biotechnol, 2004, 64, 62-69.
62 K. He, Q. Yin, A. Liu, S. Echigo, S. Itoh and G. Wu, Enhanced anaerobic degradation of amide pharmaceuticals by dosing ferroferric oxide or anthraquinone-2, 6-disulfonate, Journal of Water Process Engineering, 2017, 18, 192-197.
63 A. Kumar and H. M. Jena, Preparation and characterization of high surface area activated carbon from Fox nut (Euryale ferox) shell by chemical activation with H3PO4, Results Phys, 2016, 6, 651-658.
64 M. Thommes, K. Kaneko, A. V Neimark, J. P. Olivier, F. Rodriguez-Reinoso, J. Rouquerol and K. S. W. Sing, Physisorption of gases, with special reference to the evaluation of surface area and pore size distribution (IUPAC Technical Report), 2015, 87, 1051-1069.
65 E.-S. Chan, T.-K. Lim, W.-P. Voo, R. Pogaku, B. T. Tey and Z. Zhang, Effect of formulation of alginate beads on their mechanical behavior and stiffness, Particuology, 2011, 9, 228-234.
66 W.-P. Voo, C.-W. Ooi, A. Islam, B.-T. Tey and E.-S. Chan, Calcium alginate hydrogel beads with high stiffness and extended dissolution behaviour, Eur Polym J, 2016, 75, 343-353.
67 P. Rayment and M. F. Butler, Investigation of ionically crosslinked chitosan and chitosan-bovine serum albumin beads for novel gastrointestinal functionality, J Appl Polym Sci, 2008, 108, 2876-2885.
68 P. Del Gaudio, P. Colombo, G. Colombo, P. Russo and F. Sonvico, Mechanisms of formation and disintegration of alginate beads obtained by prilling, Int J Pharm, 2005, 302, 1-9.
69 A. Nasrullah, A. H. Bhat, A. Naeem, M. H. Isa and M. Danish, High surface area mesoporous activated carbon-alginate beads for efficient removal of methylene blue, Int J Biol Macromol, 2018, 107, 1792-1799.
70 S. K. Bajpai and S. Sharma, Investigation of swelling/degradation behaviour of alginate beads crosslinked with Ca²⁺ and Ba²⁺ ions, React Funct Polym, 2004, 59, 129-140.
71 P. Naskar, D. Chakraborty, A. Mondal, B. Das and A. Samanta, Immobilization of α-amylase in calcium alginate-gum odina (CA-GO) beads: An easily recoverable and reusable support, Int J Biol Macromol, 2024, 258, 129062.
72 H. H. Tønnesen and J. Karlsen, Alginate in Drug Delivery Systems, Drug Dev Ind Pharm, 2002, 28, 621-630.
73 J. D. Hem, W. H. Cropper and U.S. G.P.O., Survey of ferrous-ferric chemical equilibria and redox potentials, 1959.
74 L. H. N. Cooper, Oxidation-Reduction Potential in Sea Water, Journal of the Marine Biological Association of the United Kingdom, 1937, 22, 167-176.
75 S. Zhu, A. Chen, Y. Chai, R. Cao, J. Zeng, M. Bai, L. Peng, J. Shao and X. Wang, Extracellular enzyme mediated biotransformation of imidacloprid by white-rot fungus Phanerochaete chrysosporium: Mechanisms, pathways, and toxicity, Chemical Engineering Journal, 2023, 472, 144798.
76 Q. Sun, S. Yin, Y. He, Y. Cao and C. Jiang, Biomaterials and Encapsulation Techniques for Probiotics: Current Status and Future Prospects in Biomedical Applications, Nanomaterials, 2023, 13, 2185.
77 B. D. Sarma, K. C. Puzari, P. Dutta and A. K. Pandey, An alginate-based encapsulation enhances shelf life and bioactivity of the entomopathogenic fungus, Metarhizium anisopliae, Egypt J Biol Pest Control, 2023, 33, 69.
78 I. Holzmeister, M. Schamel, J. Groll, U. Gbureck and E. Vorndran, Artificial inorganic biohybrids: The functional combination of microorganisms and cells with inorganic materials, Acta Biomater, 2018, 74, 17-35
79 M. V. Tuttolomondo, G. S. Alvarez, M. F. Desimone and L. E. Diaz, Removal of azo dyes from water by sol-gel immobilized Pseudomonas sp., J Environ Chem Eng, 2014, 2, 131-136.
80 W. Ghach, M. Etienne, P. Billard, F. P. A. Jorand and A. Walcarius, Electrochemically assisted bacteria encapsulation in thin hybrid sol-gel films, J Mater Chem B, 2013, 1, 1052-1059.
81 T. Mehrotra, S. Dev, A. Banerjee, A. Chatterjee, R. Singh and S. Aggarwal, Use of immobilized bacteria for environmental bioremediation: A review, J Environ Chem Eng, 2021, 9, 105920.
82 S. H. Lau, I.-C. Lin, C.-L. Su, Y.-T. Chang and W.-N. Jane, Synthesis of cross-linked magnetic chitosan beads immobilised with bacteria for aerobic biodegrading benzophenone-type UV filter, Chemosphere, 2022, 307, 136010.
83 P. Ngamsurach, N. Namwongsa and P. Praipipat, Synthesis of powdered and beaded chitosan materials modified with ZnO for removing lead (II) ions, Sci Rep, 2022, 12, 17184.
84 B. Qu and Y. Luo, Chitosan-based hydrogel beads: Preparations, modifications and applications in food and agriculture sectors-A review, Int J Biol Macromol, 2020, 152, 437-448.
85 J. Wang and S. Zhuang, Removal of various pollutants from water and wastewater by modified chitosan adsorbents, Crit Rev Environ Sci Technol, 2017, 47, 2331-2386.
86 B. Zhou, J. Duan, L. Xue, J. Zhang and L. Yang, Effect of plant-based carbon source supplements on denitrification of synthetic wastewater: focus on the microbiology, Environmental Science and Pollution Research, 2019, 26, 24683-24694.
87 Z. Si, X. Song, Y. Wang, X. Cao, Y. Zhao, B. Wang, Y. Chen and A. Arefe, Intensified heterotrophic denitrification in constructed wetlands using four solid carbon sources: Denitrification efficiency and bacterial community structure, Bioresour Technol, 2018, 267, 416-425.
88 D. S. Tanmoy, J. C. Bezares-Cruz and G. H. LeFevre, The use of recycled materials in a biofilter to polish anammox wastewater treatment plant effluent, Chemosphere, 2022, 296, 134058.
89 R. Hu, X. Zheng, T. Zheng, J. Xin, H. Wang and Q. Sun, Effects of carbon availability in a woody carbon source on its nitrate removal behavior in solid-phase denitrification, J Environ Manage, 2019, 246, 832-839.
90 F. Zhong, S. Huang, J. Wu, S. Cheng and Z. Deng, The use of microalgal biomass as a carbon source for nitrate removal in horizontal subsurface flow constructed wetlands, Ecol Eng, 2019, 127, 263-267.
91 B. J. Halaburka, G. H. Lefevre and R. G. Luthy, Evaluation of Mechanistic Models for Nitrate Removal in Woodchip Bioreactors, Environ Sci Technol, 2017, 51, 5156-5164.
92 B. J. Halaburka, G. H. LeFevre and R. G. Luthy, Quantifying the temperature dependence of nitrate reduction in woodchip bioreactors: experimental and modeled results with applied case-study, Environ. Sci.: Water Res. Technol., 2019, 5, 782-797.
93 N. Ashoori, M. Teixido, S. Spahr, G. H. LeFevre, D. L. Sedlak and R. G. Luthy, Evaluation of pilot-scale biochar-amended woodchip bioreactors to remove nitrate, metals, and trace organic contaminants from urban stormwater runoff, Water Res, 2019, 154, 1-11.
94 M. Teixidó, J. A. Charbonnet, G. H. LeFevre, R. G. Luthy and D. L. Sedlak, Use of pilot-scale geomedia-amended biofiltration system for removal of polar trace organic and inorganic contaminants from stormwater runoff, Water Res, 2022, 226, 119246.
95 H. Kim, E. A. Seagren and A. P. Davis, Engineered Bioretention for Removal of Nitrate from Stormwater Runoff, Water Environment Research, 2003, 75, 355-367.
96 H. W. Goh, N. A. Zakaria, T. L. Lau, K. Y. Foo, C. K. Chang and C. S. Leow, Mesocosm study of enhanced bioretention media in treating nutrient rich stormwater for mixed development area, Urban Water J, 2017, 14, 134-142.
97 R. Johannes, B. P. C and B. Erland, Contrasting Soil pH Effects on Fungal and Bacterial Growth Suggest Functional Redundancy in Carbon Mineralization, Appl Environ Microbiol, 2009, 75, 1589-1596.
98 R. D. Stapleton, D. C. Savage, G. S. Sayler and G. Stacey, Biodegradation of Aromatic Hydrocarbons in an Extremely Acidic Environment, Appl Environ Microbiol, 1998, 64, 4180-4184.
99 S. Huang and P. R. Jaffé, Defluorination of Perfluorooctanoic Acid (PFOA) and Perfluorooctane Sulfonate (PFOS) by Acidimicrobium sp. Strain A6, Environ Sci Technol, 2019, 53, 11410-11419.
100 W. Keenleyside, Microbiology: Canadian Edition, Pressbooks, 2019.
101 K. Watanabe, M. Manefield, M. Lee and A. Kouzuma, Electron shuttles in biotechnology, Curr Opin Biotechnol, 2009, 20, 633-641.
102 L. Zhou, T. Chi, Y. Zhou, H. Chen, C. Du, G. Yu, H. Wu, X. Zhu and G. Wang, Stimulation of pyrolytic carbon materials as electron shuttles on the anaerobic transformation of recalcitrant organic pollutants: A review, Science of The Total Environment, 2021, 801, 149696.
103 F. Aulenta, V. Di Maio, T. Ferri and M. Majone, The humic acid analogue antraquinone-2,6-disulfonate (AQDS) serves as an electron shuttle in the electricity-driven microbial dechlorination of trichloroethene to cis-dichloroethene, Bioresour Technol, 2010, 101, 9728-9733.
104 W. Zhou, X. Chen, M. Ismail, L. Wei and B. Hu, Simulating the synergy of electron donors and different redox mediators on the anaerobic decolorization of azo dyes: Can AQDS-chitosan globules replace the traditional redox mediators?, Chemosphere, 2021, 275, 130025.
105 J. Xia, Y. Li, X. Jiang, D. Chen and J. Shen, The humic substance analogue antraquinone-2, 6-disulfonate (AQDS) enhanced zero-valent iron based autotrophic denitrification: Performances and mechanisms, Environ Res, 2023, 238, 117241.
106 G. Wang, Z. Teng, X. Zhao, W. Luo, J. Liang, Y. Guo, X. Ji, W. Hu and M. Li, AQDS-mediated dissimilatory reduction of iron (hydr) oxides induces the formation of large grain vivianite: A new insight for phosphorus pollution control in sediment, J Clean Prod, 2023, 419, 138217.
107 K. L. Straub, A. Kappler and B. Schink, Enrichment and Isolation of Ferric-Iron- and Humic-Acid-Reducing Bacteria, Methods Enzymol, 2005, 397, 58-77.
108 L. Klupfel, M. Keiluweit, M. Kleber and M. Sander, Redox Properties of Plant Biomass-Derived Black Carbon (Biochar), Environ Sci Technol, 2014, 48, 5601-5611.
109 L. G. Bó, R. M. Almeida, C. M. M. Cardoso, D. G. Zavarize, S. S. Brum and A. R. V. Mendonça, Acetylsalicylic acid biosorption onto fungal-bacterial biofilm supported on activated carbons: an investigation via batch and fixed-bed experiments, Environmental Science and Pollution Research, 2019, 26, 28962-28976.
110 L. J. Waller, G. K. Evanylo, L.-A. H. Krometis, M. S. Strickland, T. Wynn-Thompson and B. D. Badgley, Engineered and Environmental Controls of Microbial Denitrification in Established Bioretention Cells, Environ Sci Technol, 2018, 52, 5358-5366.
111 A. G. Donaghue, N. Morgan, L. Toran and E. R. Mckenzie, The impact of bioretention column internal water storage underdrain height on denitrification under continuous and transient flow, Water Res, 2022, 214, 118205.
112 S. Wang, X. Lin, H. Yu, Z. Wang, H. Xia, J. An and G. Fan, Nitrogen removal from urban stormwater runoff by stepped bioretention systems, Ecol Eng, 2017, 106, 340-348.
113 J. A. Leiva, P. C. Wilson, J. P. Albano, P. Nkedi-Kizza and G. A. O'Connor, Pesticide Sorption to Soilless Media Components Used for Ornamental Plant Production and Aluminum Water Treatment Residuals, ACS Omega, 2019, 4, 17782-17790.
114 Lalhmunsiama, R. R. Pawar, S.-M. Hong, K. J. Jin and S.-M. Lee, Iron-oxide modified sericite alginate beads: A sustainable adsorbent for the removal of As(V) and Pb(II) from aqueous solutions, J Mol Liq, 2017, 240, 497-503.
115 S. Vahidhabanu, D. Karuppasamy, A. I. Adeogun and B. R. Babu, Impregnation of zinc oxide modified clay over alginate beads: a novel material for the effective removal of congo red from wastewater, RSC Adv, 2017, 7, 5669-5678.
116 J. E. Grebel, J. A. Charbonnet and D. L. Sedlak, Oxidation of organic contaminants by manganese oxide geomedia for passive urban stormwater treatment systems, Water Res, 2016, 88, 481-491.
117 J. A. Charbonnet, Y. Duan, C. M. van Genuchten and D. L. Sedlak, Chemical Regeneration of Manganese Oxide-Coated Sand for Oxidation of Organic Stormwater Contaminants, Environ Sci Technol, 2018, 52, 10728-10736.
118 A. J. Erickson, J. S. Gulliver and P. T. Weiss, Capturing phosphates with iron enhanced sand filtration, WaterRes, 2012, 46, 3032-3042.
119 A. Erickson, J. Gulliver and P. Weiss, Phosphate Removal from Agricultural Tile Drainage with Iron Enhanced Sand, Water, 2017, 9, 672.
120 R. H. Alasfar and R. J. Isaifan, Aluminum environmental pollution: the silent killer, Environmental Science and Pollution Research, 2021, 28, 44587-44597.
121 L. A. Kszos, G. W. Morris and B. K. Konetsky, Source of toxicity in storm water: Zinc from commonly used paint, Environ Toxicol Chem, 2004, 23, 12-16.
122 C. Mallikarjuna and R. R. Dash, A review on hydrodynamic parameters and biofilm characteristics of inverse fluidized bed bioreactors for treating industrial wastewater, J Environ Chem Eng, 2020, 8, 104233.
123 M. Alsubih, R. El morabet, R. A. Khan, N. A. Khan, A. R. Khan and G. Sharma, Performance evaluation of aerobic fluidized bed bioreactor coupled with tube-settler for hospital wastewater treatment, J Environ Chem Eng, 2021, 9, 105896.
124 S. Cipullo, G. Prpich, P. Campo and F. Coulon, Assessing bioavailability of complex chemical mixtures in contaminated soils: Progress made and research needs, Science of The Total Environment, 2018, 615, 708-723.
125 P. Bhatt, S. Gangola, P. Chaudhary, P. Khati, G. Kumar, A. Sharma and A. Srivastava, Pesticide induced up-regulation of esterase and aldehyde dehydrogenase in indigenous Bacillus spp., Bioremediat J, 2019, 23, 42-52.
126 A. Medić, K. Stojanović, L. Izrael-Živković, V. Beškoski, B. Lončarević, S. Kazazić and I. Karadžić, A comprehensive study of conditions of the biodegradation of a plastic additive 2,6-di-tert-butylphenol and proteomic changes in the degrader Pseudomonas aeruginosa san ai, RSC Adv, 2019, 9, 23696-23710.
127 EPA, What is Superfund, https://www.epa.gov/superfund/what-superfund, (accessed 26 Feb. 2024). 128 R. Jing, S. Fusi and B. V. Kjellerup, Remediation of Polychlorinated Biphenyls (PCBs) in Contaminated Soils and Sediment: State of Knowledge and Perspectives, Front Environ Sci, DOI: 10.3389/fenvs.2018.00079.
129 S. L. Woods, D. J. Trobaugh and K. J. Carter, Polychlorinated Biphenyl Reductive Dechlorination by 970 Vitamin B12s: Thermodynamics and Regiospecificity, Environ Sci Technol, 1999, 33, 857-863.
130 T. F. M. Rodgers, S. Spraakman, Y. Wang, C. Johannessen, R. C. Scholes and A. Giang, Bioretention Design Modifications Increase the Simulated Capture of Hydrophobic and Hydrophilic Trace Organic Compounds, Environ Sci Technol, 2024, 58, 5500-5511.

### Example 2—Bio-Active Composite Alginate Bead Media to Sustainably Remove Trace Organics and Dissolved Nutrients in Green Stormwater Infrastructure

### Materials and Methods

### Chemicals:

Neonicotinoids (imidacloprid and desnitro-imidacloprid; purity ≥95%) and acetanilide (purity ≥99%) for abiotic and biotic bench-scale experiments were purchased from Fisher Scientific Company. PFAS and PO43-. Synthetic urban stormwater with common major ions was prepared following our previously described method. (9) Optima LCMS grade solvents (acetonitrile, formic acid, water, methanol) were used for all chromatography analysis. Compositions of different BioSorp beads are shown in Table SI 1.

TABLE SI 1
Compositions of different types of BioSorp beads.

| Bead | Sodium | | Wood | | | | Drying |
|---|---|---|---|---|---|---|---|
| name | alginate | PAC | Dust | Fe-WTR | AQDS | Cross-linker | temperature |
| 0.5% SA-3% CaCl₂ | 0.5% (w/v) | 1% (w/v) | 1% (w/v) | — | — | 270.3 mM CaCl₂ | Air dried at room |
| | | | | | | | temp |
| 1% SA-3% CaCl₂(also | 1% (w/v) | 1% (w/v) | 1% (w/v) | — | — | 270.3 mM CaCl₂ | Air dried at room |
| known as PAC-WD-CaCl₂) | | | | | | | temp |
| 1.5% SA-3% CaCl₂ | 1.5% (w/v) | 1% (w/v) | 1% (w/v) | — | — | 270.3 mM CaCl₂ | Air dried at room |
| | | | | | | | temp |
| 1% SA-5% CaCl₂ | 1% (w/v) | 1% (w/v) | 1% (w/v) | — | — | 450.5 mM CaCl₂ | Air dried at room |
| | | | | | | | temp |
| PAC-WD-WTR-CaCl₂ | 1% (w/v) | 1% (w/v) | 1% (w/v) | 1% (w/v) | — | 270.3 mM CaCl₂ | Air dried at room |
| | | | | | | | temp |
| PAC-WD-WTR-AQDS-CaCl₂ | 1% (w/v) | 1% (w/v) | 1% (w/v) | 1% (w/v) | 0.1% | 270.3 mM CaCl₂ | Air dried at room |
| | | | | | | | temp |
| PAC-WD-WTR-AQDS-FeCl3 | 1% (w/v) | 1% (w/v) | 1% (w/v) | 1% (w/v) | 0.1% | 270.5 mM FeCl3 | Air dried at room |
| | | | | | | | temp |
| WD-CaCl₂ | 1% (w/v) | — | 1% (w/v) | — | — | 270.3 mM CaCl₂ | Air dried at room |
| | | | | | | | temp |
| WD-WTR-CaCl₂ | 1% (w/v) | — | 1% (w/v) | 1% (w/v) | — | 270.3 mM CaCl₂ | Air dried at room |
| | | | | | | | temp |
| PAC-WD-WTR-AQDS-FeCl₃ | 1% (w/v) | 1% (w/v) | 1% (w/v) | 1% (w/v) | — | 270.3 mM FeCl₃ | Air dried at room |
| | | | | | | | temp |
| WD-WTR-FeCl₃ | 1% (w/v) | — | 1% (w/v) | 1% (w/v) | — | 270.3 mM FeCl₃ | Air dried at room |
| | | | | | | | temp |
| T40 | 1% (w/v) | 1% (w/v) | 1% (w/v) | — | — | 270.3 mM CaCl₂ | Oven dried at 40 deg |
| | | | | | | | C. |
| T70 | 1% (w/v) | 1% (w/v) | 1% (w/v) | — | — | 270.3 mM CaCl₂ | Oven dried at 70 deg |
| | | | | | | | C. |

### Bead Performance Experimental Designs:

To assess the performance of the BioSorp beads for contaminant removal, we have selected imidacloprid (neonicotinoid insecticide), desnitro-imidacloprid (common imidacloprid metabolite), one long chain PFAS (PFOA), two short chain PFASs (PFBS and PFBA), and phosphate (dissolved P nutrient) as model stormwater runoff contaminants.

Imidacloprid is one of the most common urban-use neonicotinoid insecticides that has higher water solubility (log Kow=0.6) and lower affinity towards soil particles. (8,10,11) Imidacloprid often undergoes a microbial transformation in the environment and produces a metabolite, desnitro-imidacloprid, which is significantly more toxic to mammals (more than 300 times). (12) Mammalian nicotinic acetylcholine receptor (α4β2 nAChR) inhibitory concentrations (IC₅₀) for imidacloprid and desnitro-imidacloprid are 2600 nM and 8.2 nM, respectively. (13) Thus, removal of imidacloprid and their metabolite from urban stormwater runoff is a growing concern.

Increasing PFAS concentrations in water sources and their nature of extreme persistence in the environment have been a matter of great concern. US EPA's recent announcement of Maximum Contaminant Levels (MCL) for PFAS in drinking water has shed new light towards PFAS removal from stormwater runoff. (14) As stormwater runoff replenish a variety of drinking water treatment plant influent sources, any sort of PFAS removal in stormwater infrastructure eventually improves drinking water quality and reduces treatment costs.

Why dissolved phosphate (80-90%) is more difficult to remove than particulate phosphate (10-20%). Prestigiacomo et al. found 10-20% of particulate phosphorus was bioavailable, compared to more than 90% of dissolved phosphorus being bioavailable. Bioavailable phosphorus in the particulate fraction increased somewhat with time after sampling, but never exceeded 30%.

Abiotic Sorption Experiments: BioSorp beads with different compositions were used for various abiotic sorption experiments. For the abiotic sorption experiments, neonicotinoids were dissolved in synthetic stormwater, whereas PFAS and dissolved phosphorus sorption experiments were performed in deionized water.

Isotherm experiments: Isotherm experiments with varied concentrations (10, 15, 20, 25, and 30 mg/L) of imidacloprid and desnitro-imidacloprid were conducted in 125 mL glass serum vials where PAC-wood flour beads (1% sodium alginate-1% PAC-1% wood flour-3% calcium chloride) were added as adsorbent. In these batch test type experiments, 100 mg beads were added into 100 mL neonic solutions. The serum vials were sealed using rubber stoppers and were kept inside a cardboard box to eliminate any chanckirke of photoreaction. The box was then kept onto a platform shaker for the whole experimental duration. The vials were taken out from the shaker from time to time while taking the samples. Similar experimental and sampling procedures were also followed in all the following experiments. The maximum imidacloprid and desnitro-imidacloprid sorption capacities of the PAC-wood flour beads were calculated using the Langmuir equation shown below.

$${q}_{e} = \frac{{q}_{max} {K}_{L} {C}_{e}}{1 + {K}_{L} {C}_{e}} \qquad ( Eq 1 )$$

Here, qₑ=equilibrium sorbed concentration (mg/g), qₘₐₓ=maximum sorption capacity of the sorbent (mg/g), K_{L}=Langmuir constant (L/mg), Cₑ=equilibrium aqueous concentration.

### LC Method for PFOA, PFBA, and PFBS:

Column: Acquity BEH C18 (2.1×50 mm, 1.7 μm)
Column Temp: 25 C
Flow Rate: 0.3 ml/min
Isocratic flow 15% Solvent A/85% Solvent B for 5 min
Solvent A: 10 mM Ammonium Acetate in Water, pH 5.0
Solvent B: 10 mM Ammonium Acetate in 80:20 Methanol/Water
Sample Temp: 10 C

### Mass Spec Methods:

Waters MassLynx software used for data acquisition
Waters TargetLynx software used for peak detection and integration
Electrospray Ionization in negative ion mode
   Source Parameters include—
Capillary Voltage: 3.80 KV
Desolvation Gas: Nitrogen
Desolvation Gas Temp.: 400 C
Desolvation Gas Flow: 400 L/hr
Cone Gas Flow: 50 L/hr
Collision Gas: Argon
   MRM Transition with Cone Voltage and Collision Energy—

In TargetLynx software, peaks from the TIC (or combination of all MRM transitions listed above) was used for peak detection and integration.

PFOA

| MRM Transition | Cone Voltage (V) | Collision Energy (eV) |
|---|---|---|
| 412.94→169.11 | 8 | 16 |
| 412.94→219.03 | 8 | 14 |
| 412.94→369.06 | 8 | 8 |

PFBS

| MRM Transition | Cone Voltage (V) | Collision Energy (eV) |
|---|---|---|
| 298.92→78.89 | 6 | 32 |
| 298.92→82.88 | 6 | 24 |
| 298.92→98.89 | 6 | 26 |

PFBA

| MRM Transition | Cone Voltage (V) | Collision Energy (eV) |
|---|---|---|
| 212.95→168.97 | 16 | 8 |
| 212.95→180.88 | 16 | 10 |

### Results and Discussion

Removal of TOrCs and Dissolved Nutrients Via Abiotic Sorption onto BioSorp Beads.
Neonicotinoid Insecticide and Acetanilide Sorption onto BioSorp Beads:

Compositions of BioSorp Beads affect abiotic sorption of imidacloprid and its common environmental metabolite, desnitro-imidacloprid (FIG. A1). It was observed that the predicted maximum imidacloprid sorption capacities were similar for PAC_WF_CaCl₂) beads and PAC_WF_WTR_CaCl₂) beads (26.39 mg/g vs 25.52 mg/g; p>0.9999); however, there was a significant difference between maximum imidacloprid sorption capacities for PAC_WF_WTR_CaCl₂) beads vs. PAC_WF_WTR_FeCl₃ beads (25.52 mg/g vs 18.52 mg/g; p=0.0015**). Conversely, all three types of the beads exhibited similar maximum desnitro-imidacloprid sorption (PAC_WF_CaCl₂) beads vs. PAC_WF_WTR_CaCl₂) beads (14.96 mg/g vs 11.14 mg/g; p=0.1402); PAC_WF_WTR_CaCl₂) beads vs. PAC_WF_WTR_FeCl₃ beads [11.14 mg/g vs 13.04 mg/g; p=0.7285]). Beads made only with wood flour and iron water treatment residuals did not sorb any imidacloprid and desnitro-imidacloprid, which indicates that neonicotinoids are only sorbed onto the PAC present in the beads. Black carbons have multiple active adsorption sites consisting of single and double carbon bonds, hydroxyl groups, metal oxides and hydroxides, aldehyde groups, carboxyl groups, aromatic carbon skeletons with delocalized pi electrons, and other oxygen-rich functional groups, and can thus sorb various trace organics and ionic contaminants. (15, 16)

Similar to the bead composition, the partial charge distribution of the molecules also affects maximum neonicotinoid sorption capacities. When the maximum sorption capacities of the beads were normalized to the amount of PAC used in the recipes, a slight decrease was observed in imidacloprid sorption capacities and a slight increase in desnitro-imidacloprid sorption capacities from raw PAC capacity values for both Ca²⁺ alginate and Fe³⁺ alginate bead types (FIG. A1). In PAC_WF_CaCl₂) beads, ˜25% decrease was observed in imidacloprid sorption capacity (FIG. SI 4) and ˜22% increase in desnitro-imidacloprid sorption capacity. A similar decrease in imidacloprid sorption capacity was also observed in PAC_WF_WTR_CaCl₂) beads and in PAC_WF_WTR_FeCl₃ beads (˜11% vs. ˜ 37%). On the other hand, desnitro-imidacloprid sorption capacities were increased both in PAC_WF_WTR_CaCl₂) beads (˜9%) and PAC_WF_WTR_FeCl₃ beads (˜25%). The molecular partial charge distribution also impacted the neonicotinoid sorption kinetics of the beads. The average adsorption rate constant of desnitro-imidacloprid was 1.75 times higher than that of imidacloprid (0.28 day⁻¹ vs 0.16 day⁻¹).

It was hypothesized that the opposing sorption effects between imidacloprid and desnitro-imidacloprid are likely due to the interactions between alginate and neonicotinoid pharmacophores. These types of interactions were also observed for imidacloprid and desnitro-imidacloprid sorption onto granular activated carbon (GAC) and carbon nano tubes (CNTs). (12) GAC contains many localized basic functional groups and favors imidacloprid sorption than desnitro-imidacloprid sorption at neutral pH. (12) Furthermore, evidence of the effects of partial charge distribution was bolstered when neonicotinoid sorption capacities were measured in functionalized and non-functionalized CNTs. Compared to non-functionalized CNTs, carboxylic acid-functionalized CTNs (contain more partial negative charges) sorbed more desnitro-imidacloprid. Contrarily, amine-functionalized CNTs (contain more partial positive charges) sorbed less desnitro-imidacloprid than non-functionalized CNTs. The opposite effects were observed in the case of imidacloprid sorption. Alginate gel contains an abundant amount of negatively charged hydroxyl and carboxyl groups at neutral pH (pKa of alginic acid is 3), which can be crosslinked with positively charged Ca²⁺ or Fe³⁺ ions. (17-19) These abundant negative charges on the alginate structures might have experienced some attractive forces with the electron-donating amine group present on desnitro-imidacloprid, which eventually increased the sorption capacity. Desnitro-imidacloprid is a more concerning contaminant due to its higher toxicity towards mammals and BioSorp beads improve its sorption in engineered black carbon system.

For all three types of beads, there were significant differences between imidacloprid and desnitro-imidacloprid sorption capacities (PAC_WF_CaCl₂) beads [p<0.0001****], PAC_WF_CaCl₂) beads [p<0.0001****], and PAC_WF_WTR_FeCl₃ beads [p=0.0191*]). For both neonicotinoids, there were also likely some sorption capacity losses, because some of the sorption sites present on the PAC surface might have been occupied during the alginate encapsulation process. Trivalent iron produces denser and stronger crosslinks with alginate than divalent calcium, which retards contaminant diffusion through the alginate crosslinks. (20, 12) This was likely the reason behind the significant difference between PAC_WD_WTR_CaCl₂) beads vs. PAC_WD_WTR_FeCl₃ beads (p=0.0015**) for imidacloprid sorption. Additionally, Fe³⁺ bonds with both polyguluronate (GG) and polymannuronate (GM) groups of alginates in Fe³⁺ alginate beads, whereas Ca²⁺ bonds only with polyguluronate (GG) groups of alginates in Ca²⁺alginate beads.²¹ As a result, Fe³⁺ alginate bead might contain more partial negative charges (due to the higher number of carboxylic ions present), which might aid desnitro-imidacloprid sorption. Similar interactions between partial charges were also reported in a previous study, where the release of cationic drugs from alginate bead system were slower than the release of anionic drugs. (22)

The presence of black carbon (PAC) played a role in both imidacloprid and desnitro-imidacloprid sorption onto BioSorp Beads and no neonicotinoid sorption was achieved via the beads that did not contain any black carbon (FIG. A2)
a. From the isotherm experiments, it was observed that BioSorp beads made with 1% sodium alginate, 1% PAC, 1% wood flour, and 3% calcium chloride (PAC_WD_CaCl₂) beads) sorbed up to 29.05 mg/g imidacloprid and 16.83 mg/g desnitro-imidacloprid (using Langmuir equation.
b. The maximum neonicotinoid sorption capacities were predicted from the kinetics experiments using the modified Langmuir equation. Calcium-alginate beads made with PAC, wood flour, and iron water treatment residuals (PAC_WD_WTR_CaCl₂) beads) have shown maximum imidacloprid sorption capacity of 25.52 mg/g and maximum desnitro-imidacloprid sorption capacity of 11.14 mg/g. On the other hand, iron-alginate beads made with PAC, wood flour, and iron water treatment residuals (PAC_WD_WTR_FeCl₃ beads) have shown maximum imidacloprid sorption capacity of 18.52 mg/g and maximum desnitro-imidacloprid sorption capacity of 13.04 mg/g.

Change in bead alginate or crosslinker concentrations also impacts neonicotinoid sorption kinetics of the beads, whereas bead drying temperature did not have any affects.
a. With increasing sodium alginate and/or calcium chloride concentrations in the recipe, denser bead structures are formed. This change in bead structures also affects imidacloprid and desnitro-imidacloprid sorption kinetics. If the alginate and/or crosslinker concentrations are increased, the first order reaction rates are decreased for both neonicotinoids. When the sodium alginate concentration is increased from 0.5% to 1.5%, imidacloprid sorption rate becomes 0.13 day⁻¹ from 0.22 day⁻¹ and desnitro-imidacloprid sorption rate becomes 0.17 day 1 from 0.21 day⁻¹. If the calcium chloride concentration is increased to 5% from 3%, imidacloprid sorption rate becomes 0.16 day⁻¹ from 0.19 day⁻¹ and desnitro-imidacloprid sorption rate becomes 0.15 day⁻¹ from 0.18 day⁻¹.
b. There were no impacts of bead drying temperatures on the neonic sorption kinetics. Three kinds of beads were tested (dried at room temperature, 40° C., and 70° C.) and all of them have demonstrated similar sorption kinetics for imidacloprid and desnitro-imidacloprid.
   Dissolved Phosphorus Sorption onto BioSorp Beads:

Fe³⁺ alginate beads were better at sorbing dissolved phosphorus than Ca²⁺ alginate beads and the sorption capacity increased with the increase in available Fe³⁺ ions present in the beads.
a. The maximum phosphate sorption capacities in WF_WTR_FeCl₃ beads and FeWTR FeCl₃ beads were 42.12 mg/g and 38.88 mg/g, respectively. On the other hand, the maximum phosphate sorption capacities in WF_WTR_CaCl₂) beads and PAC_WF_WTR_CaCl₂) beads were 13.01 mg/g and 8.25 mg/g, respectively. Because we did not observe any phosphate sorption onto PAC_WF_CaCl₂) and WF_CaCl₂) beads, it was hypothesized that PAC, WF, and Ca²⁺ crosslinker did not aid phosphate sorption. It was very likely that the FeWTR, present in the Ca²⁺ alginate beads contributed to the phosphate sorption capacity. However, Isik et al. (2021) (23) showed that their calcium alginate beads could sorb 3.27 mg/g phosphate.
b. Ferric ions in Fe³⁺ alginate beads can strongly bond with phosphate and thus, remove dissolved phosphorus from aqueous phase. (24)
c. FeWTR might also aid phosphate sorption. FeWTR contains different iron oxides and hydroxides which can form strong bonds with phosphate ions. (25-27) Iron oxide amended sands (i.e., sand with iron filings) had been used previously to remove dissolved phosphorus from stormwater. (28) Ligand exchange reaction and hydroxide exchange reaction between FeWTR and phosphate might have played the role to sorb the dissolved phase phosphorus. (29) Previously, Wang et al. (2011) (30) also used ferric alum water treatment residuals (Fe—Al-WTRs) to sorb dissolved phosphorus (maximum sorption capacity=45.42 mg/g).
d. Like in Ca-beads, the presence of FeWTR in Fe-beads also has increased phosphate sorption capacities.
e. We did not observe any significant phosphate sorption onto PAC and wood flour.
   PFAS Sorption onto BioSorp Beads:

BioSorp Beads removed a higher amount of long-chain PFAS (PFOA) than short-chain PFAS (PFBA and PFBS) from synthetic stormwater runoff (FIG. A5).
a. Most of the PAC-free Ca²⁺ alginate beads did not sorb any PFAS (exception: 0.39 mg PFBS/g sorption onto 1% sodium alginate-1% Wood flour-1% FeWTR-3% CaCl₂) Beads), which represents that PFAS sorption was dominated via sorption onto the PAC present on the Ca²⁺ alginate beads. Black carbon materials, such as PAC can effectively sorb long-chain PFAS via hydrophobic interactions, whereas short-chain PFAS removal is dominated by electrostatic interactions. (31) On the other hand, it was observed that 6.91 mg PFOA/g and 0.84 mg PFBS/g sorption onto PAC-free Fe³⁺ alginate beads, which represents that the Fe³⁺ ions in the bead crosslinkers helped with PFOA sorption.
b. The FeWTR in the beads might also aid PFAS sorption because FeWTR might contain many iron oxides and hydroxides. Metal oxides and hydroxides sorb PFAS via electrostatic interactions, ligand exchange with —COO⁻ or —SO₃⁻ groups present in PFAS structures, hydrophobic interactions, and hydrogen bonds. (31, 32)
c. PFAS removal is abiotic sorption, whereas some of the other organic contaminants re biodegraded by the encapsulated organisms.

### Coupled Sorption and Fungal Degradation of Acetanilide Via BioSorp Beads.

Both Ca²⁺ alginate and Fe³⁺ alginate beads could remove tire wear compound, acetanilide from synthetic stormwater via coupled sorption and biodegradation.
a. Ca²⁺ alginate beads and Fe³⁺ alginate beads were prepared encapsulating two types of white rot fungi (T. versicolor and P. ostreatus) to investigate acetanilide removal in fungal treatments (BioSorp Beads containing alive white rot fungi), autoclaved controls (fungi containing BioSorp Beads were autoclaved at 121 deg C. for 45 minutes), and azide inhibited controls (10 mM sodium azide were spiked into the synthetic stormwater).
b. We observed the highest acetanilide removal in fungal treatments and the lowest in autoclaved controls for all the four fungi-BioSorp Bead, representing evidence of biodegradation along with sorption (Ca²⁺ alginate and Fe³⁺ alginate beads, containing 2 types of white rot fungi). It was hypothesized that autoclaved fungi beads represented abiotic conditions to the closest (most of the encapsulated fungi were dead, if not all). It is likely that fungal biofilms blocked some of the bead sorption sites and beads containing no fungi would have sorbed a higher amount of acetanilide than the autoclaved fungi-Ca²⁺/Fe³⁺ alginate beads. Because biodegradation is needed for sustained contaminant removal and it can renew sorption capacities (33), BioSorp Beads containing alive fungi are better candidates for bioretention cells.
c. Furthermore, it was found that alginate encapsulation protects the fungi from harsh environmental conditions. Sodium azide is regularly used for microbial inhibition. (34) However, acetanilide removal in the azide inhibited controls was close to the fungal treatments. This phenomenon represents that the encapsulated fungi were alive and actively biodegrading acetanilide ever after the addition of sodium azide into the synthetic stormwater. Hence, it was hypothesized that alginate encapsulation might also help the fungi to be active in field bioretention conditions.

TABLE SI 2
Comparison of the maximum sorption capacities of different bead
types for imidacloprid and its metabolite, desnitro-imidacloprid,

| | Mean | 95.00% CI | Below | | Adjusted |
|---|---|---|---|---|---|
| Tukey's multiple comparisons test | Diff [symbol] | of dif [symbol] | threshold? | Summary | P Value |
| PAC_WD_CaCl2:Imidacloprid vs. | 11.46 | 6.763 to 16.16 | Yes | **** | <0.0001 |
| PAC_WD_CaCl2:Desnitro-imidacloprid | | | | | |
| PAC_WD_CaCl2:Imidacloprid vs. | 0.14 | −4.557 to 4.837 | No | ns | <0.9999 |
| PAC_WD_WTR_CaCl2:Imidacloprid | | | | | |
| PAC_WD_CaCl2:Imidacloprid vs. PAC_{—} | 15.27 | 10.58 to 19.97 | Yes | **** | <0.0001 |
| WD_WTR_CaCl2:Desnitro-imidacloprid | | | | | |
| PAC_WD_CaCl2:Imidacloprid vs. | 7.827 | 3.129 to 12:52 | Yes | ** | 0.0013 |
| PAC_WD_WTR_FeCl3:Imidacloprid | | | | | |
| PAC_WD_CaCl2:Imidacloprid vs. PAC_{—} | 13.32 | 8.623 to: 18.02 | Yes | **** | <0.0001 |
| WD_WTR_FeCl3:Desnitro-imidacloprid | | | | | |
| PAC_WD_CaCl2:Desnitro-imidacloprid | −11.32 | −16.02 to −6.623 | Yes | **** | <0.0001 |
| vs. PAC_WD_WTR_CaCl2:Imidacloprid | | | | | |
| PAC_WD_CaCl2:Desnitro-imidacloprid vs. | 3.813 | −0.8839 to 8.511 | No | ns | 0.1402 |
| PAC_WD_WTR_CaCl2:Desnitro-imidacloprid | | | | | |
| PAC_WD_CaCl2:Desnitro-imidacloprid | −3.633 | −8.331 to 1.064 | No | ns | 0.1711 |
| vs. PAC_WD_WTR_FeCl3:Imidacloprid | | | | | |
| PAC_WD_CaCl2:Desnitro-imidacloprid vs | 1.86 | −2.837 to 6.557 | No | ns | 0.7643 |
| PAC_WD_WTR_FeCl3:Desnitro-imidacloprid | | | | | |
| PAC_WD_WTR_CaCl2:Imidacloprid vs. | 15.13 | 10.44 to 19.83 | Yes | **** | <0.0001 |
| PAC_WD_WTR_CaCl2:Desnitro-imidacloprid | | | | | |
| PAC_WD_WTR_CaCl2:Imidacloprid vs. | 7.687 | 2.989 to 12.38 | Yes | ** | 0.0015 |
| PAC_WD_WTR_FeCl3:Imidacloprid | | | | | |
| PAC_WD_WTR_CaCl2:Imidacloprid vs. | 13.18 | 8.483 to 17.88 | Yes | **** | <0.0001 |
| PAC_WD_WTR_FeCl3:Desnitro-imidacloprid | | | | | |
| PAC_WD_WTR_CaCl2:Desnitro-imidacloprid | −7.447 | −12.14 to −2.749 | Yes | ** | 0.0019 |
| vs. PAC_WD_WTR_FeCl3:Imidacloprid | | | | | |
| PAC_WD_WTR_CaCl2:Desnitro-imidacloprid vs. | −1.953 | −6.651 to 2.744 | No | ns | 0.7285 |
| PAC_WD_WTR_FeCl3:Desnitro-imidacloprid | | | | | |
| PAC_WD_WTR_FeCl3:Imidacloprid vs. | 5.493 | 0.7961 to 10.19 | Yes | * | 0.0191 |
| PAC_WD_WTR_FeCl3:Desnitro-imidacloprid | | | | | |

[symbol]

indicates data missing or illegible when filed

TABLE SI 3
First order reaction rates for imidacloprid and desnitro-
imidacloprid sorption onto different PAC-Wood dust beads.
[The compositions of these beads are in Table SI 1]

| | Imidacloprid | | Desnitro-imidacloprid | |
|---|---|---|---|---|
| | 1st order | | 1st order | |
| | Reaction | | Reaction | |
| Bend type | rate (1/day) | r² | rate (1/day) | r² |
| 0.5% SA-3% | 0.22 ± 0.01 | 0.97 ± 0.01 | 0.21 ± 0.006 | 0.96 ± 0.006 |
| CaCl₂ | | | | |
| 1% SA-3% | 0.19 ± 0.01 | 0.99 ± 0.002 | 0.18 ± 0.01 | 0.97 ± 0.003 |
| CaCl₂ | | | | |
| 1.5% SA-3% | 0.13 ± 0.003 | 0.98 ± 0.01 | 0.17 ± 0.003 | 0.95 ± 0.02 |
| CaCl₂ | | | | |
| 1% SA-5% | 0.16 ± 0.004 | 0.99 ± 0.003 | 0.15 ± 0.009 | 0.95 ± 0.003 |
| CaCl₂ | | | | |
| T40 | 0.21 ± 0.002 | 0.99 ± 0.004 | 0.18 ± 0.01 | 0.96 |
| T70 | 0.21 ± 0.02 | 0.97 ± 0.004 | 0.17 ± 0.003 | 0.93 ± 0.003 |

TABLE SI 4
Phosphate sorption onto various BioSorp beads (Calcium-alginate and iron-alginate beads).

| | maximum phosphate | | |
|---|---|---|---|
| | sorption capacity | | |
| Types of bead | (mg/g) | adsorption rate constant, K [symbol] (day⁻¹) | r² |
| WD-WTR-CaCl₂ | 13.01 | 0.37 | 0.83 |
| PAC_WD_WTR_CaCl₂ | 8.25 | 0.27 | 0.97 |
| WD_CaCl₂ | 2.10 at day 18 | *did not follow Lang [symbol] adsorption model | — |
| PAC_WD_CaCl₂ | 0.88 at day 18 | * did not follow Lang [symbol] adsorption model | — |
| WD-WTR-FeCl₃ | 42.12 | 0.99 | 0.89 |
| WTR_FeCl₃ | 38.88 | 1.33 | 0.96 |
| WD_FeCl₃ | 34.03 | 1.52 | 0.95 |
| PAC_WD_FeCl₃ | 29.55 | 0.37 | 0.92 |
| PAC_FeCl₃ | 24.98 | 0.29 | 0.89 |
| PAC_WD_WTR_FeCl₃ | 17.03 | 17.03 | 0.98 |

[symbol]

indicates data missing or illegible when filed

### BIBLIOGRAPHY

(1) Lapointe, M.; Rochman, C. M.; Tufenkji, N. Sustainable Strategies to Treat Urban Runoff Needed. Nat Sustain 2022. https://doi.org/10.1038/s41893-022-00853-4.
(2) Rodak, C. M.; Moore, T. L.; David, R.; Jayakaran, A. D.; Vogel, J. R. Urban Stormwater Characterization, Control, and Treatment. Water Environment Research 2019, 91 (10), 1034-1060. https://doi.org/10.1002/wer.1173.
(3) Goonetilleke, A.; Lampard, J.-L. Chapter 3-Stormwater Quality, Pollutant Sources, Processes, and Treatment Options; Sharma, A. K., Gardner, T., Begbie, D. B. T.-A. to W. S. U. D., Eds.; Woodhead Publishing, 2019; pp 49-74. https://doi.org/https://doi.org/10.1016/B978-0-12-812843-5.00003-4.
(4) Gunawardena, J. M. A.; Liu, A.; Egodawatta, P.; Ayoko, G. A.; Goonetilleke, A.; Gunawardena, J. M. A.; Liu, A.; Egodawatta, P.; Ayoko, G. A.; Goonetilleke, A. Predicting Stormwater Quality Resulting from Traffic Generated Pollutants. In Influence Of Traffic And Land Use On Urban Stormwater Quality: Implications For Urban Stormwater Treatment Design; 2018; pp 55-69. https://doi.org/10.1007/978-981-10-5302-3_4.
(5) Masoner, J. R.; Kolpin, D. W.; Cozzarelli, I. M.; Barber, L. B.; Burden, D. S.; Foreman, W. T.; Forshay, K. J.; Furlong, E. T.; Groves, J. F.; Hladik, M. L.; Hopton, M. E.; Jaeschke, J. B.; Keefe, S. H.; Krabbenhoft, D. P.; Lowrance, R.; Romanok, K. M.; Rus, D. L.; Selbig, W. R.; Williams, B. H.; Bradley, P. M. Urban Stormwater: An Overlooked Pathway of Extensive Mixed Contaminants to Surface and Groundwaters in the United States. Environ Sci Technol 2019, 53 (17), 10070-10081. https://doi.org/10.1021/acs.est.9b02867.
(6) Jia, Q.; Li, B.; Li, B.; Cai, Y.; Yuan, X. Experiments and Simulation of Adsorption Characteristics of Typical Neonicotinoids in Urban Stream Sediments. Environmental Science and Pollution Research 2023, 30 (31), 76992-77005. https://doi.org/10.1007/s11356-023-27025-X.
(7) Xiong, J.; Wang, Z.; Ma, X.; Li, H.; You, J. Occurrence and Risk of Neonicotinoid Insecticides in Surface Water in a Rapidly Developing Region: Application of Polar Organic Chemical Integrative Samplers. Science of The Total Environment 2019, 648, 1305-1312. https://doi.org/https://doi.org/10.1016/j.scitotenv.2018.08.256.
(8) Webb, D. T.; Nagorzanski, M. R.; Powers, M. M.; Cwiertny, D. M.; Hladik, M. L.; LeFevre, G. H. Differences in Neonicotinoid and Metabolite Sorption to Activated Carbon Are Driven by Alterations to the Insecticidal Pharmacophore. Environ Sci Technol 2020, 54 (22), 14694-14705. https://doi.org/10.1021/acs.est.0c04187.
(9) Wolfand, J. M.; LeFevre, G. H.; Luthy, R. G. Metabolization and Degradation Kinetics of the Urban-Use Pesticide Fipronil by White Rot Fungus Trametes Versicolor. Environ Sci Process Impacts 2016, 18 (10), 1256-1265. https://doi.org/10.1039/C6EM00344C.
(10) Zhang, P.; Ren, C.; Sun, H.; Min, L. Sorption, Desorption and Degradation of Neonicotinoids in Four Agricultural Soils and Their Effects on Soil Microorganisms. Science of The Total Environment 2018, 615, 59-69.
https://doi.org/https://doi.org/10.1016/j.scitotenv.2017.09.097.
(11) Hladik, M. L.; Kolpin, D. W.; Kuivila, K. M. Widespread Occurrence of Neonicotinoid Insecticides in Streams in a High Corn and Soybean Producing Region, USA. Environmental Pollution 2014, 193, 189-196. https://doi.org/https://doi.org/10.1016/j.envpol.2014.06.033.
(12) Webb, D. T.; Nagorzanski, M. R.; Powers, M. M.; Cwiertny, D. M.; Hladik, M. L.; LeFevre, G. H. Differences in Neonicotinoid and Metabolite Sorption to Activated Carbon Are Driven by Alterations to the Insecticidal Pharmacophore. Environ Sci Technol 2020, 54 (22), 14694-14705. https://doi.org/10.1021/acs.est.0c04187.
(13) Tomizawa, M.; Casida, J. E. Selective Toxicity Of Neonicotinoids Attributable To Specificity Of Insect And Mammalian Nicotinic Receptors. Annu Rev Entomol 2003, 48 (1), 339-364. https://doi.org/10.1146/annurev.ento.48.091801.112731.
(14) US EPA. Proposed PFAS National Primary Drinking Water Regulation. https://www.epa.gov/sdwa/and-polyfluoroalkyl-substances-pfas.
(15) Mopoung, S.; Moonsri, P.; Palas, W.; Khumpai, S. Characterization and Properties of Activated Carbon Prepared from Tamarind Seeds by KOH Activation for Fe(III) Adsorption from Aqueous Solution. The Scientific World Journal 2015, 2015, 415961. https://doi.org/10.1155/2015/415961.
(16) Hassan, M.; Liu, Y.; Naidu, R.; Parikh, S. J.; Du, J.; Qi, F.; Willett, I. R. Influences of Feedstock Sources and Pyrolysis Temperature on the Properties of Biochar and Functionality as Adsorbents: A Meta-Analysis. Science of The Total Environment 2020, 744, 140714. https://doi.org/https://doi.org/10.1016/j.scitotenv.2020.140714.
(17) Wang, B.; Wan, Y.; Zheng, Y.; Lee, X.; Liu, T.; Yu, Z.; Huang, J.; Ok, Y. S.; Chen, J.; Gao, B. Alginate-Based Composites for Environmental Applications: A Critical Review. Crit Rev Environ Sci Technol 2019, 49 (4), 318-356. https://doi.org/10.1080/10643389.2018.1547621.
(18) Shalapy, A.; Zhao, S.; Zhang, C.; Li, Y.; Geng, H.; Ullah, S.; Wang, G.; Huang, S.; Liu, Y. Adsorption of Deoxynivalenol (DON) from Corn Steep Liquor (CSL) by the Microsphere Adsorbent SA/CMC Loaded with Calcium. Toxins. 2020. https://doi.org/10.3390/toxins12040208.
(19) Garbayo, I.; León, R.; Vílchez, C. Diffusion Characteristics of Nitrate and Glycerol in Alginate. Colloids Surf B Biointerfaces 2002, 25 (1), 1-9. https://doi.org/https://doi.org/10.1016/S0927-7765 (01) 00287-9.
(20) Malektaj, H.; Drozdov, A. D.; deClaville Christiansen, J. Mechanical Properties of Alginate Hydrogels Cross-Linked with Multivalent Cations. Polymers (Basel) 2023, 15 (14). https://doi.org/10.3390/polym15143012.
(21) Massana Roquero, D.; Othman, A.; Melman, A.; Katz, E. Iron (Iii)-Cross-Linked Alginate Hydrogels: A Critical Review. Mater Adv 2022, 3 (4), 1849-1873. https://doi.org/10.1039/DIMA00959A.
(22) Tønnesen, H. H.; Karlsen, J. Alginate in Drug Delivery Systems. Drug Dev Ind Pharm 2002, 28 (6), 621-630. https://doi.org/10.1081/DDC-120003853.
(23) Isik, Z.; Saleh, M.; Dizge, N. Adsorption Studies of Ammonia and Phosphate Ions onto Calcium Alginate Beads. Surfaces and Interfaces 2021, 26, 101330. https://doi.org/https://doi.org/10.1016/j.surfin.2021.101330.
(24) Siwek, H.; Bartkowiak, A.; Włodarczyk, M.; Sobecka, K. Removal of Phosphate from Aqueous Solution Using Alginate/Iron (III) Chloride Capsules: A Laboratory Study. Water Air Soil Pollut 2016, 227 (11), 427. https://doi.org/10.1007/s11270-016-3128-0.
(25) Ippolito, J. A.; Barbarick, K. A.; Elliott, H. A. Drinking Water Treatment Residuals: A Review of Recent Uses. J Environ Qual 2011, 40 (1), 1-12. https://doi.org/https://doi.org/10.2134/jeq2010.0242.
(26) Lambert, N.; Van Aken, P.; Van den Broeck, R.; Dewil, R. Adsorption of Phosphate on Iron-Coated Sand Granules as a Robust End-of-Pipe Purification Strategy in the Horticulture Sector. Chemosphere 2021, 267, 129276.
https://doi.org/https://doi.org/10.1016/j.chemosphere.2020.129276.
(27) Likus, M.; Komorowska-Kaufman, M.; Pruss, A.; Zych, Ł.; Bajda, T. Iron-Based Water Treatment Residuals: Phase, Physicochemical Characterization, and Textural Properties. Materials 2021, 14 (14). https://doi.org/10.3390/ma14143938.
(28) Erickson, A. J.; Gulliver, J. S.; Weiss, P. T. Capturing Phosphates with Iron Enhanced Sand Filtration. Water Res 2012, 46 (9), 3032-3042. https://doi.org/https://doi.org/10.1016/j.watres.2012.03.009.
(29) Gibbons, M. K.; Gagnon, G. A. Understanding Removal of Phosphate or Arsenate onto Water Treatment Residual Solids. J Hazard Mater 2011, 186 (2), 1916-1923. https://doi.org/https://doi.org/10.1016/j.jhazmat.2010.12.085.
(30) Wang, C.; Guo, W.; Tian, B.; Pei, Y.; Zhang, K. Characteristics and Kinetics of Phosphate Adsorption on Dewatered Ferric-Alum Residuals. Journal of Environmental Science and Health, Part A 2011, 46 (14), 1632-1639. https://doi.org/10.1080/10934529.2011.623643.
(31) Smaili, H.; Ng, C. Adsorption as a Remediation Technology for Short-Chain per- and Polyfluoroalkyl Substances (PFAS) from Water—a Critical Review. Environ Sci (Camb) 2023, 9 (2), 344-362. https://doi.org/10.1039/D2EW00721E
(32) Lin, H.; Wang, Y.; Niu, J.; Yue, Z.; Huang, Q. Efficient Sorption and Removal of Perfluoroalkyl Acids (PFAAs) from Aqueous Solution by Metal Hydroxides Generated in Situ by Electrocoagulation. Environ Sci Technol 2015, 49 (17), 10562-10569. https://doi.org/10.1021/acs.est.5b02092.
(33) Portmann, A. C.; LeFevre, G. H.; Hankawa, R.; Werner, D.; Higgins, C. P. The Regenerative Role of Biofilm in the Removal of Pesticides from Stormwater in Biochar-Amended Biofilters. Environ. Sci.: Water Res. Technol. 2022, 8 (5), 1092-1110. https://doi.org/10.1039/DIEW00870F.
(34) Wiener, E. A.; LeFevre, G. H. White Rot Fungi Produce Novel Tire Wear Compound Metabolites and Reveal Underappreciated Amino Acid Conjugation Pathways. Environ Sci Technol Lett 2022, 9 (5), 391-399. https://doi.org/10.1021/acs.estlett.2c00114.

### Example 3—Efficacy of BioSorp Bead Sorption

Because encapsulation of sorptive materials can decrease surface area, as was observed herein, BioSorp Beads sorption efficacy was investigated. Imidacloprid was selected as a representative hydrophilic TOrC and sorption experiments were conducted with several types of abiotic beads (i.e., did not contain any fungi) to quantify BioSorp Bead's contaminant capture capability. Imidacloprid is a neonicotinoid insecticide (representative highly polar TOrC; log K_{ow}=0.57) that is frequently applied in urban gardens, lawns, and for treating household pets and thus commonly detected in urban stormwater. (1) Here, imidacloprid was spiked in synthetic stormwater to create a solution of contaminated runoff (imidacloprid concentration=˜ 30 mg/L). 100 mL of the prepared contaminated stormwater was transferred in serum vials and 100 mg of BioSorp Beads was added to each vial. After sealing the vial tops, the vials were maintained on a platform shaker in the dark for ˜two weeks (until equilibrium is reached, based on prior work (1)). The modified Langmuir equation (1) was used to predict the maximum sorption capacities for two representative BioSorp Beads in this proof-of concept experiment.

Encapsulation of the sorptive materials did decrease sorption capacity, but much less than the decreased surface area would predict. Imidacloprid sorption capacity for the PAC_WF_WTR_CaCl₂) bead was 25.52 mg/g (bead recipe: 1% alginate, 1% PAC, 1% WE, 1% FeWTR, and 270.3 mM CaCl₂) and 18.52 mg/g for the PAC_WF_WTR_FeCl₃ bead (bead recipe: 1% alginate, 1% PAC, 1% WF, 1% FeWTR, and 270.3 mM FeCl₂). The imidacloprid sorption capacity was determined for this raw PAC (112 mg/g) in one of the previous studies. (1) When the sorption capacities were normalized with respect to the mass of PAC in the beads, sorption capacities of PAC_WF_WTR_CaCl₂) beads and PAC_WF_WTR_FeCl₃ beads were 99.53 mg/g and 70.93 mg/g, respectively. Thus, even though the average surface area loss in the beads was ˜87% (with respect to raw PAC), average PAC normalized imidacloprid sorption capacity loss was only ˜23% (FIG. SI 4). Some of PAC's sorption sites may have been blocked during encapsulation, thereby limiting imidacloprid sorption. The lower imidacloprid sorption onto PAC_WF_WTR_FeCl₃ beads compared to PAC_WF_WTR_CaCl₂) beads may relate to the denser crosslinked hydrogel formation in Fe beads than in Ca beads.

### BIBLIOGRAPHY

1 D. T. Webb, M. R. Nagorzanski, M. M. Powers, D. M. Cwiertny, M. L. Hladik and G. H. LeFevre, Differences in Neonicotinoid and Metabolite Sorption to Activated Carbon Are Driven by Alterations to the Insecticidal Pharmacophore, Environ Sci Technol, 2020, 54, 14694-14705.

### Example 4—BioSorp Beads to Remove Nitrate/Encapsulating Sorptive Materials and Anaerobic Microorganisms in Composite Alginate Bead Geomedia to Remove Nitrate from Water

Nitrate, an inorganic dissolved nitrogen nutrient, is one of the major contaminants of concern in urban areas. High levels of nitrate in drinking water can severely harm human health. Currently, the EPA has set a maximum contaminant level (MCL) for nitrate in drinking water at 10 mg/L. Excess nitrate in urban runoff deteriorates water quality in rivers, streams, and lakes via eutrophication and can result in aquatic biodiversity loss, fish death, restrictions in surface water dependent recreational, agricultural, and industrial activities, and increased water treatment costs. Because conventional stormwater management systems, such as bioretention cells are generally designed to capture particle bound contaminants and nitrate is highly water soluble, most conventional stormwater management systems remove little to no nitrate from stormwater runoff. Thus, there is a clear need for bioretention amendments so that nitrate is captured and biodegraded by stormwater management in situ. Modified BioSorp Beads containing nitrate sorbing materials and denitrifying microorganisms can potentially remove nitrate from stormwater runoff via coupled sorption and subsequent biodegradation. In this work, we demonstrated the versatility of the BioSorp Bead geomedia platform technology by adapting sorptive materials and encapsulated biodegrading microorganisms, but with the consistent overall goal of capturing highly hydrophilic, dissolved stormwater contaminants during rapid stormwater infiltration coupled with subsequent biodegradation to sustain removal during antecedent dry periods in green stormwater infrastructure.

### Introduction

Urban areas typically generate intense stormwater runoff during precipitation and/or snow-melt events due to high abundance of impervious surfaces (e.g., buildings, parking lots, roads), lack of vegetation, and low groundwater infiltration rates. (1) Because urban stormwater runoff contains a wide variety of environmental contaminants, untreated stormwater runoff often causes severe water pollution in receiving waterbodies. (2) Nitrate, an inorganic dissolved nitrogen nutrient, is one of the major contaminants of concerns in urban areas. (2) Excess nitrate in urban runoff deteriorates water quality in rivers, streams, and lakes (2) via eutrophication (3-6) and can result in aquatic biodiversity loss, fish death, restrictions in surface water dependent recreational, agricultural, and industrial activities, and increased water treatment costs. (4, 7) The US EPA reports that 44% of US rivers and streams were impacted by elevated nitrogen nutrient loads. (8) Elevated nitrate concentrations also encourage harmful algal blooms. (5,9) Nitrate in urban stormwater runoff can originate from a wide variety of human induced and environmental sources. Atmospheric deposition of NOx (emitted from vehicular and industrial exhausts) is a major source. (10) Application of nitrogen fertilizers in urban landscapes (e.g., lawns, golf courses, city parks), decomposing organic waste and soil organic matters (e.g., pet waste, plant materials), malfunctioning sewer and septic systems are also notable sources urban nitrate pollutions. (11, 12) Sustained mitigation of nitrate pollution in urban stormwater runoff can be improved through incorporation of green stormwater infrastructure (GSI) practices, including bioretention cells, bioswales, and constructed wetlands if these practices are optimized for nitrate removal (12)

Bioretention cells at GSI practice being increasingly implemented in urban areas to capture and biodegrade various stormwater relevant contaminants in situ and to increase groundwater recharge¹³ while improving the human urban environment. (14) Conventionally, bioretention cells are designed with gravel, sand, soil, compost, and mulch layers (also support vegetation growth) that facilitate fast infiltration rates to reduce extended flooding (15) Conventional bioretention cells are generally very effective at removing particulate contaminants such as pathogens, suspended solids, some heavy metals, and some nutrients. (16) Nevertheless, dissolved nutrients and trace organic contaminants (TOrCs) can pass through conventional bioretention cells getting little to no treatment (17-19) Thus, various bioretention geomedia amendments with sorptive materials (e.g., black carbon materials (biochar, GAC, PAC), iron-based sorptive materials, manganese oxide coated sand) and design modifications were recommended in literature to improve dissolved nutrient and TOrC removal (20-22) Because nitrate is poorly sorbed onto soil or particles, nitrate removal in conventional bioretention cell is heavily dependent on the presence of anaerobic microorganisms in the system (i.e., nitrate removal via denitrification). (23, 24) As such, bioretention design modifications are often recommended to promote anaerobic microorganism growth by increasing bioretention saturation zones. (25, 26) Saturated underdrain layer (also often referred as submerged zone) significantly decreases nitrate load during inter-storm dry periods. (27-29) Saturated underdrain layers are generally constructed below the main filter layers of bioretention cells and are filled with large gravels or crushed stones to increase bioretention water storage capacity. The submerged conditions are often maintained either by installing an upturned underdrain or by positioning the drainage pipes above the saturated layers. (30) Saturated layer maintains anoxic conditions that promote denitrifying microbe growth and thus, increasing nitrate removal. Addition of external carbon sources in saturated layer were also recommended to increase denitrifying activities (30, 31) Nevertheless, the majority of the bioretention cells do not contain saturated underdrain layers and post-construction modification of existing bioretention cells to add saturated zone requires major physical modification. Huang et al. (32) reviewed 128 different bioretention cells around the world and only 21% of the reviewed cells contained saturated underdrain layers. Furthermore, bioretention cells containing high quantities of compost in media or decomposing plant materials can also leach dissolved nutrients into the outflow during rainfall events. (33) Bioretention amendments with biochar, water treatment residual, iron-rich sorptive media, and eggshells can minimize phosphate leaching; however, these approaches yield virtually no decrease in nitrate leaching. (34, 35) Hence, introducing nitrate sorbing amendments and denitrification promoting microorganisms in bioretention systems can provide sustained nitrate removal from stormwater runoff.

Provided herein above is a novel composite alginate geomedia (called BioSorp Beads) which encapsulates sorptive materials (powdered activated carbon (PAC), iron-based water treatment residuals), contaminant-degrading organisms (white rot fungi), and growth substrate (wood flour) to bioaugment stormwater management systems and to promote coupled sorption and biodegradation of stormwater-relevant trace organic contaminants. (36, 37) Because bioretention cells are designed to minimize extended flooding by facilitating rapid infiltration rates, the presence of sorptive materials in the system can improve effluent stormwater quality by capturing the contaminants during stormwater infiltration even when contact time between water and media is limited. Contaminant capture during infiltration with subsequent biotransformation during the inter-storm periods decouples the bioretention cell's hydraulic retention time (HRT) and chemical residence time (CRT), thereby renewing the system's sorption capacity. Additionally, encapsulation is known to improve microorganism activity and protect microbes against harsh environmental conditions to increase microorganism viability. (38) Indeed, coupled sorption and biodegradation of a tire wear compound (acetanilide) via the developed BioSorp Beads (contained white rot fungi as the biodegrading organism) was observed even in the presence of a known fungal inhibitor (sodium azide). (37, 39)

To expand the composite alginate geomedia BioSorp Bead technology to additional stormwater contaminants, the adaptability of BioSorp Beads was investigated using different microorganisms and sorptive materials for dissolved nutrient removal. Indeed, the original BioSorp Beads demonstrated successful removal of dissolved phosphate; (37) however, nitrate is also a pressing stormwater challenge. The specific objective of this research was to develop newly adapted BioSorp Beads to capture (via sorption) and subsequently biodegrade stormwater nitrate by encapsulating sorptive materials (PAC, anion exchange resin), minerals (iron oxide, iron sulfide), growth substrate (wood flour), and denitrifying microorganisms (grown from the anoxic zone of a wastewater treatment plant). Anion exchange resin (AER) was encapsulated to enhance the nitrate sorption capacity of BioSorp Bead as AER holds strong affinity towards nitrate at environmentally relevant pH. (40) PAC was included in the bead system despite the fact that PAC is likely to show little to no nitrate sorption (41) because PAC is an excellent sorptive material capable of capturing a wide variety of stormwater relevant trace organic contaminants. (42) Wood flour (sanding dust, a valorized wood waste product) was included as an organic carbon source and electron donor for the encapsulated denitrifiers, enhancing microbial viability. (43) Finally, addition of magnetite (iron oxide, Fe₃O₄) and iron sulfide (FeS) can potentially sustain long term microbial denitrification by working as inorganic electron donors. (44, 45) It was hypothesized that encapsulation of denitrifying microorganisms, growth substances, and sorptive materials, in the modified BioSorp Beads could provide simultaneous stormwater nitrate sorption and denitrification. Ultimately, the versatility of the BioSorp Bead geomedia platform technology was demonstrated by adapting sorptive materials and encapsulated biodegrading microorganisms, but with the consistent overall goal of capturing highly hydrophilic, dissolved stormwater contaminants during rapid stormwater infiltration coupled with subsequent biodegradation to sustain removal during antecedent dry periods in green stormwater infrastructure.

### Materials and Methods

### Chemicals

Anion exchange resin (AER) (AmberChrom 1X2 chloride form, 100-200 mesh) and iron (II,III) oxide were purchased from Sigma Aldrich and Strem Chemicals, respectively. Powdered activated carbon (PAC) and iron (II) sulfide were purchased from Thermo Scientific. Wood flour (sanding dust residual) were purchased from Shannon's Sawmill (Syracuse, New York, USA). Sodium alginate, sodium nitrate, LB broth base, riboflavin, sodium acetate, calcium chloride, and ferric chloride were purchased from Fisher Scientific. Synthetic urban stormwater runoff was prepared as described above.

### Bead Preparation

The earlier described BioSorp Bead recipe was customized to cater to stormwater/agricultural or landscape runoff water nitrate removal by encapsulating different sorptive materials, minerals, and denitrifying microorganisms. Sludge was collected from the anoxic zone of a wastewater treatment plant in North Liberty, Iowa, United States. To enrich anaerobic microorganisms in a liquid culture, 1 mL anaerobic sludge was mixed in 999 mL LB broth and the culture was maintained in anoxic condition until the OD600 reached around 0.8. To prepare the beads, 1 g PAC, 1 g iron (II,III) oxide, 1 g iron (II) sulfide, 1 g wood flour, 5 g AER, and 66.7 mL anaerobic microorganism culture in every 33.3 mL 3% (w/v; made in DI water) sodium alginate solution were mixed. After that, a peristaltic pump was used to add the alginate mixture dropwise onto a crosslinking solution (3% (w/v) CaCl₂) (270.3 mM) solution; prepared in DI water) to instantaneously form the composite alginate beads. Finally, the wet beads were air dried on wax paper for 2-3 days after gently washing the freshly prepared beads several times with DI water. The dried beads were stored in the refrigerator at 4 degrees Celsius (dried bead diameter: ˜ 3-4 mm).

### Rationale for Bead Materials

Anion exchange resin (AER) was encapsulated to enhance the nitrate sorption capacity of BioSorp Bead because AER holds strong affinity towards nitrate at environmentally relevant pH. PAC was included in the bead system despite the fact that PAC is likely to show little to no nitrate sorption because PAC is an excellent sorptive material capable of capturing a wide variety of stormwater relevant trace organic contaminants. Wood flour (sanding dust, a valorized wood waste product) was included as an organic carbon source and electron donor for the encapsulated denitrifiers, enhancing microbial viability. Finally, the addition of magnetite (iron oxide, Fe₃O₄) and iron sulfide (FeS) can sustain long term microbial denitrification by working as inorganic electron donors. It was hypothesized that encapsulation of denitrifying microorganisms, growth substances, and sorptive materials, in the modified BioSorp Beads could provide simultaneous stormwater nitrate sorption and denitrification. Ultimately, the versatility of the BioSorp Bead geomedia platform technology was demonstrated by adapting sorptive materials and encapsulated biodegrading microorganisms, but with the consistent overall goal of capturing highly hydrophilic, dissolved stormwater contaminants during rapid stormwater infiltration coupled with subsequent biodegradation to sustain removal during antecedent dry periods in green stormwater infrastructure.

### Results

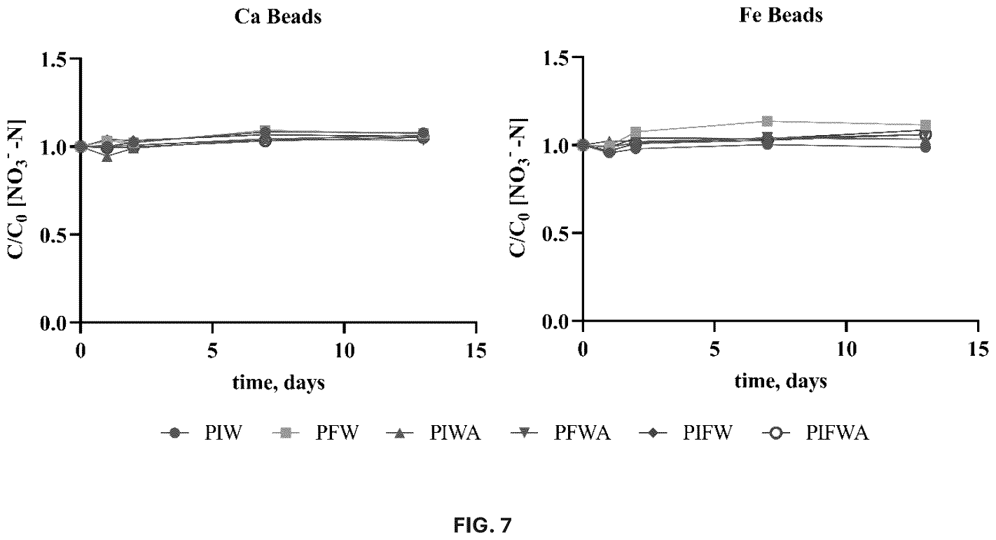

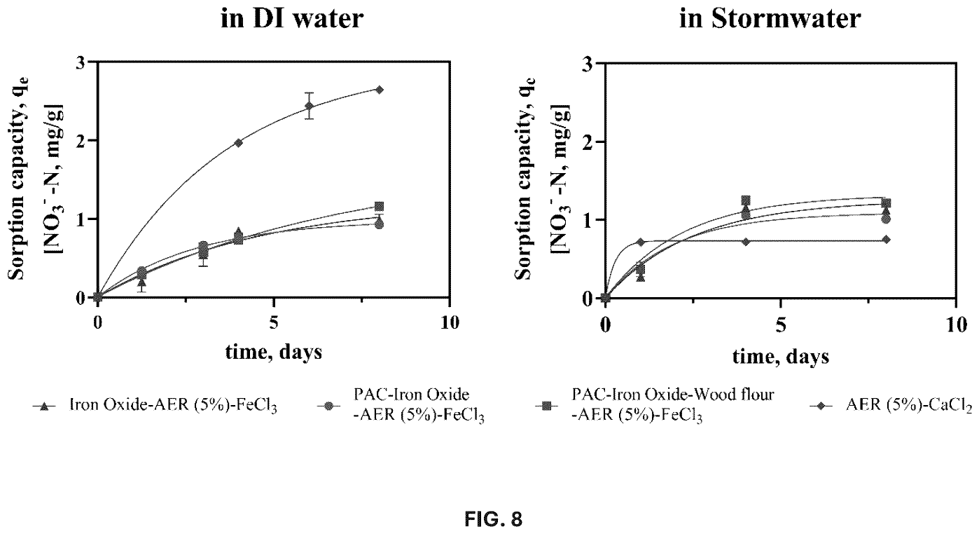

Beads containing no or small amount of (0.1%) anion exchange resin (AER) did not sorb any nitrate (FIG. 7). BioSorp Beads containing 5% AER demonstrated nitrate sorption capacity (FIG. 7). Nitrate sorption capacities of abiotic BioSorp Bead ranged from 0.74 to 1.36 mg/g nitrate-nitrogen in synthetic stormwater and 0.99 to 3.11 mg/g nitrate-nitrogen in DI water. Thus, 5% AER was added to the bead recipe.

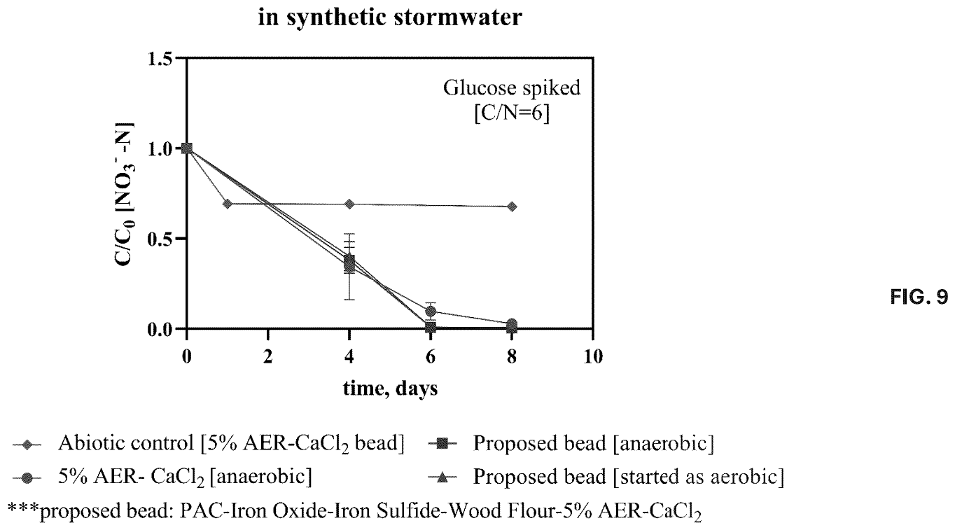

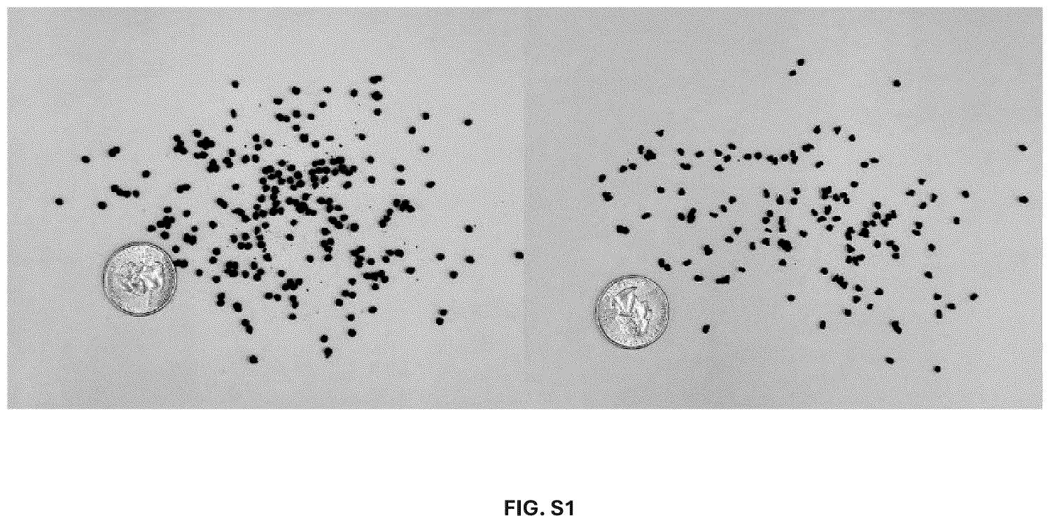

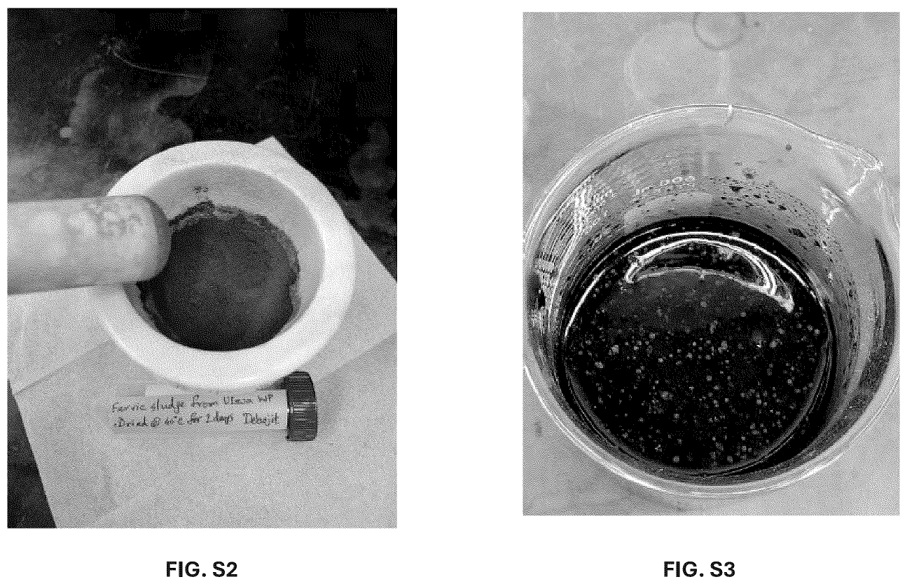

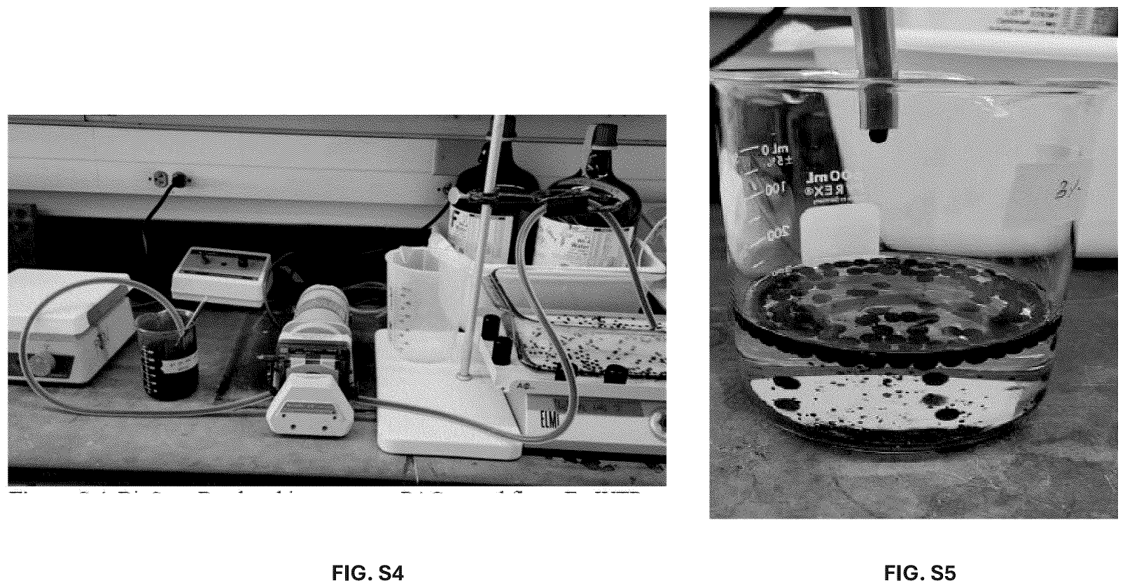

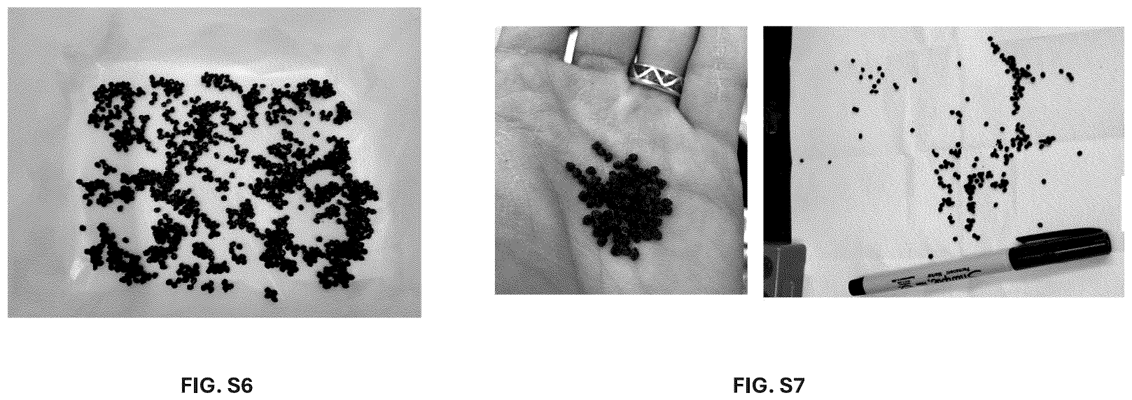

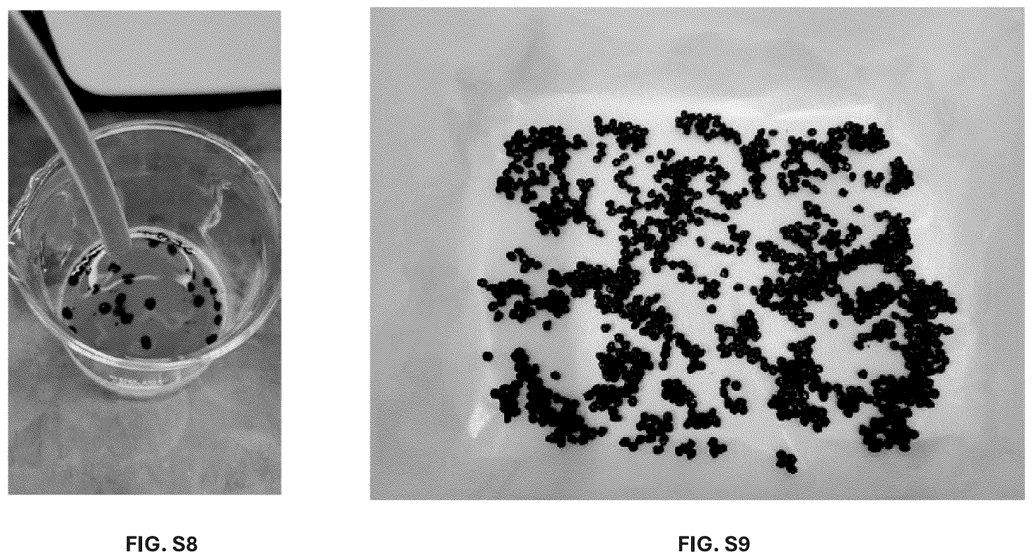

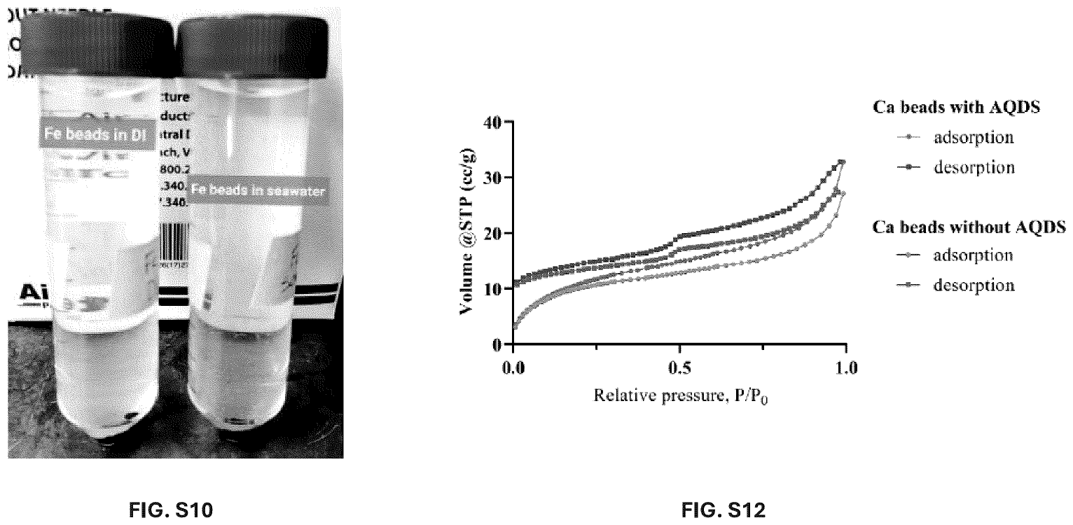

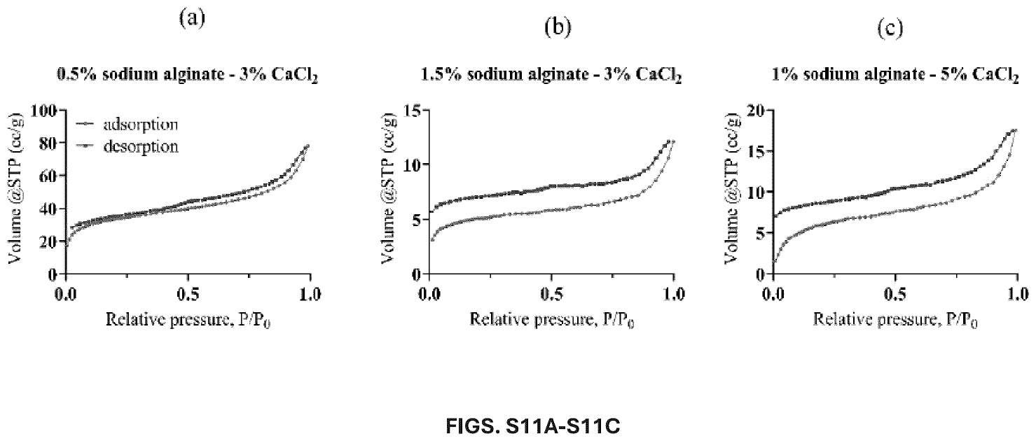

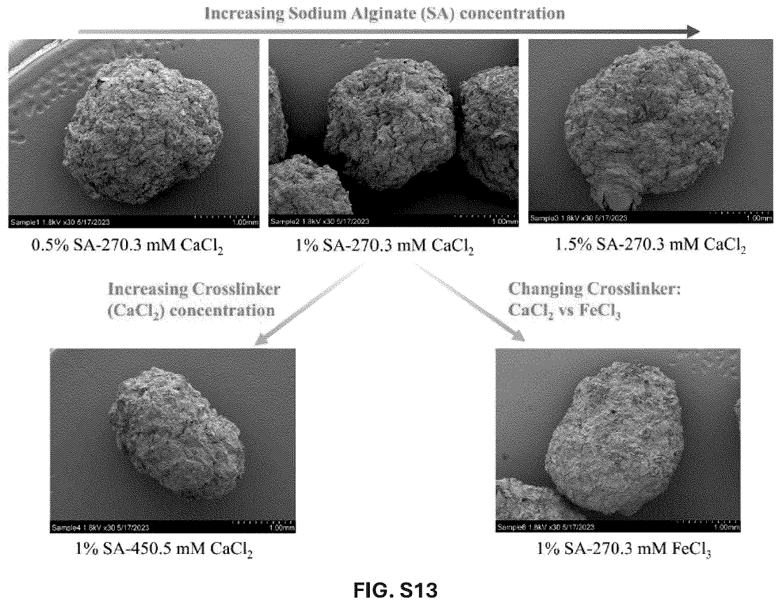

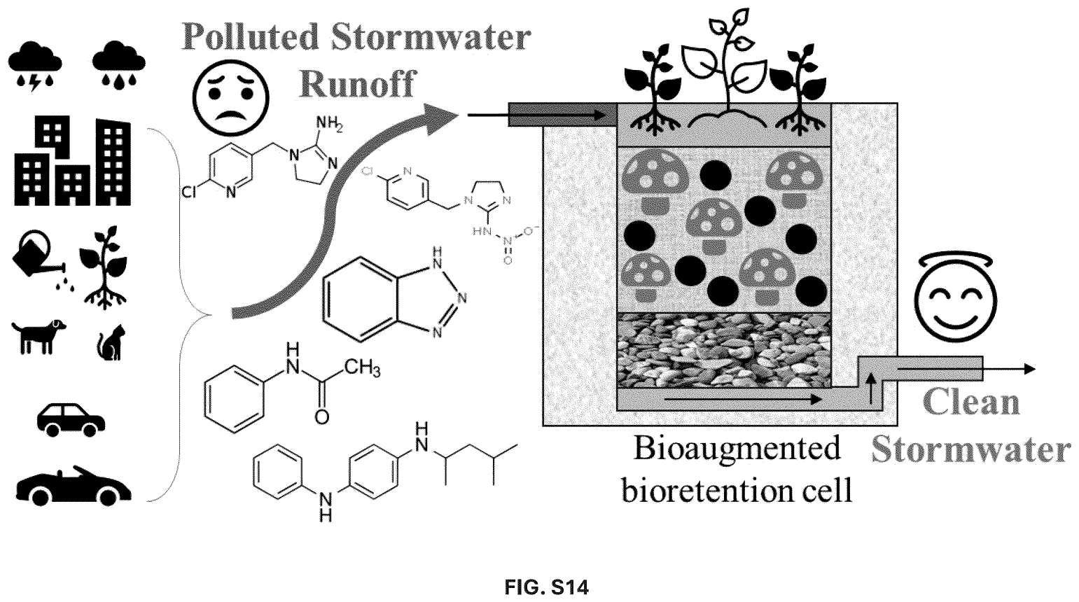

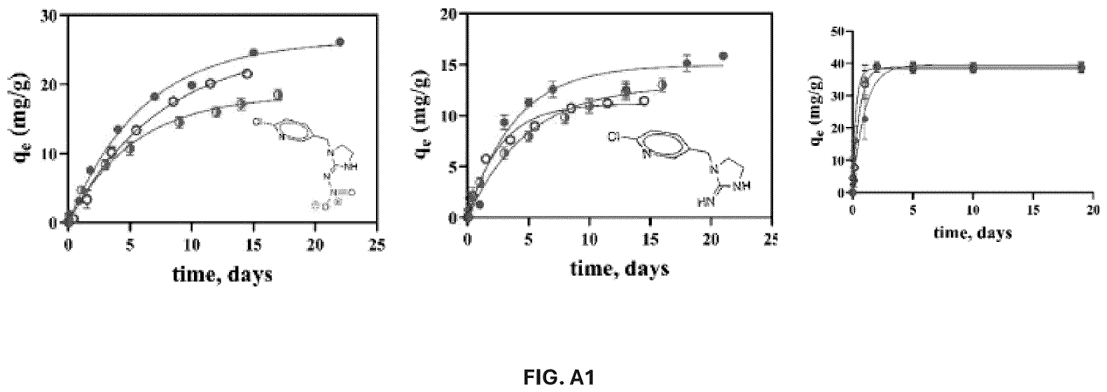

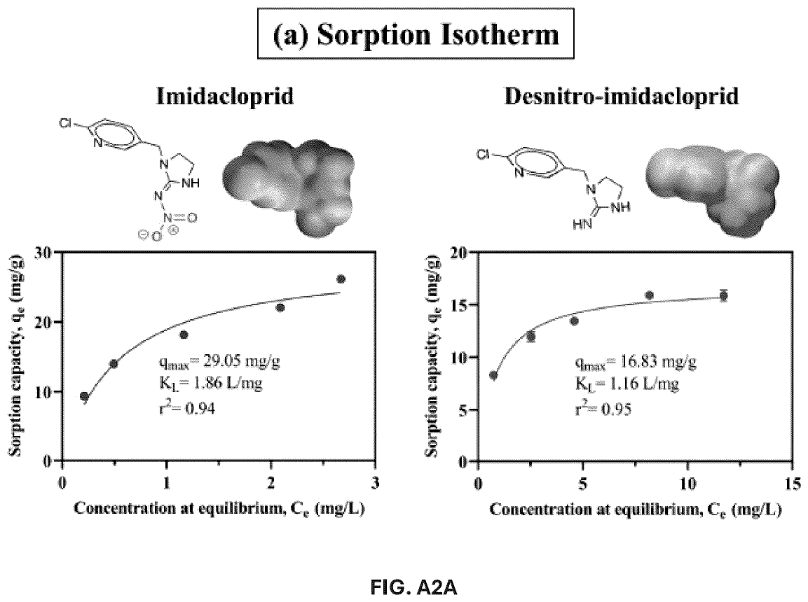

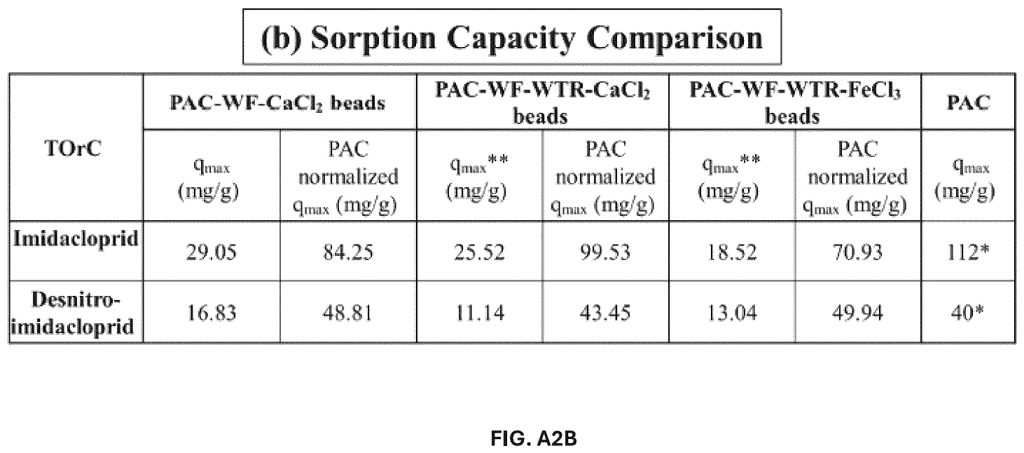

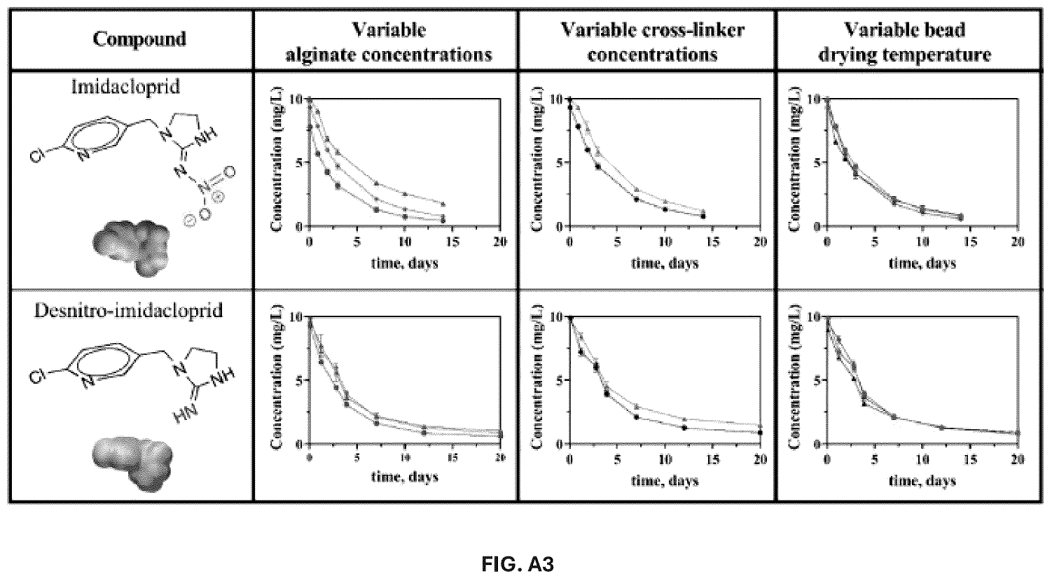

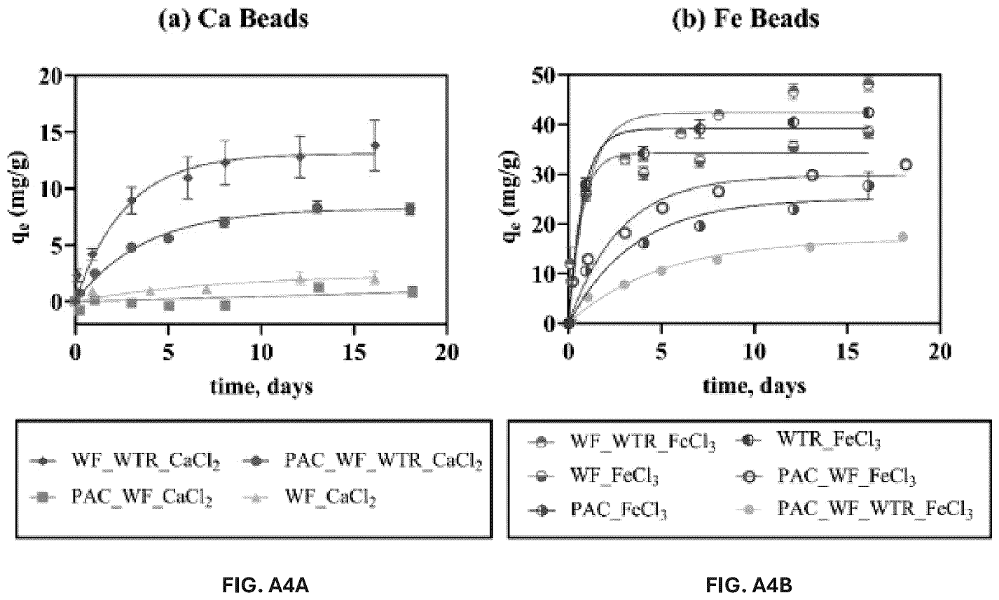

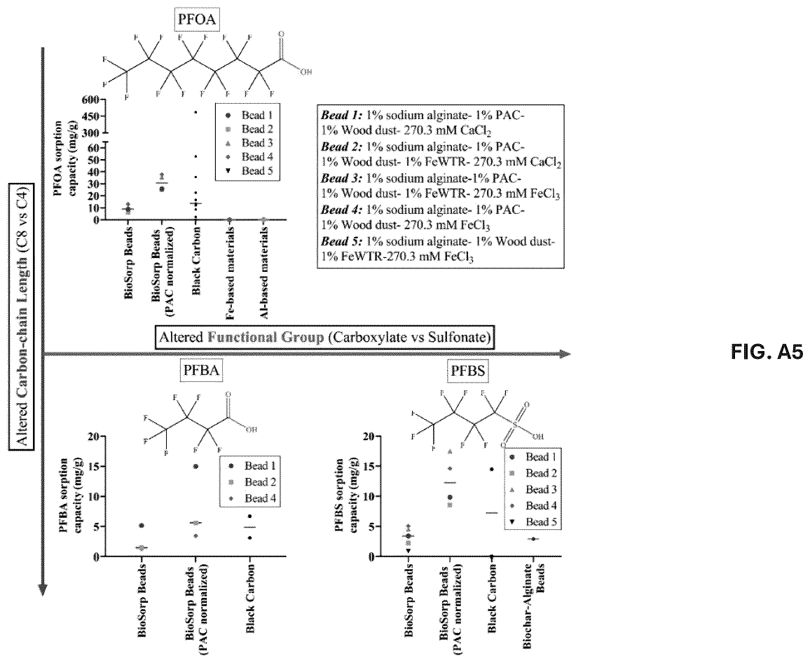

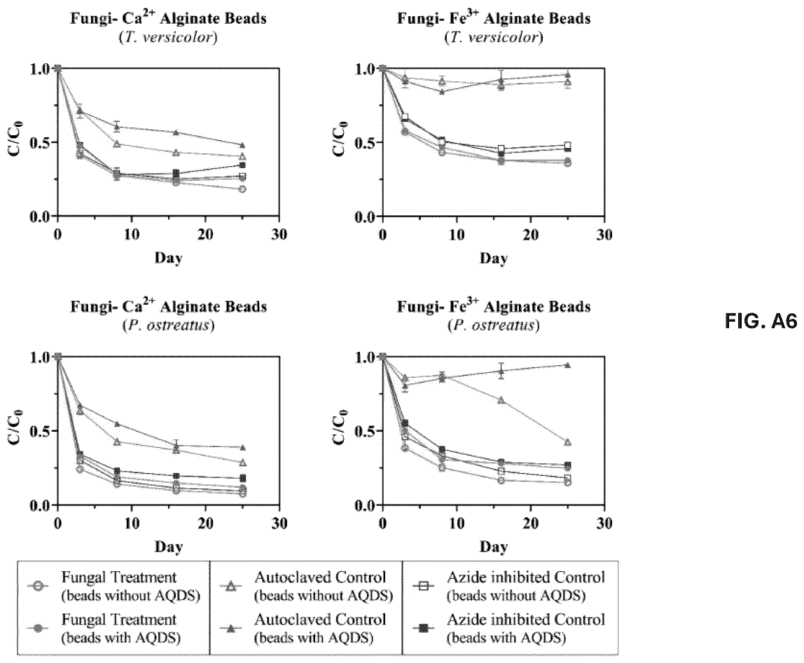

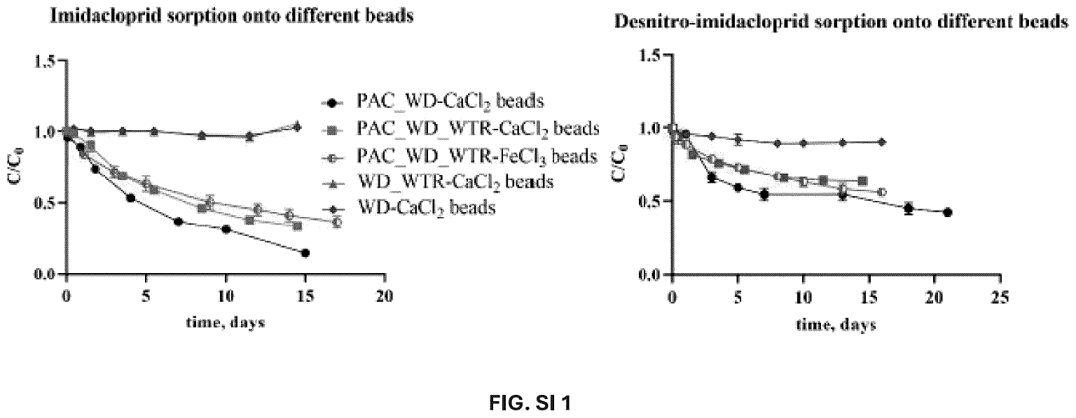

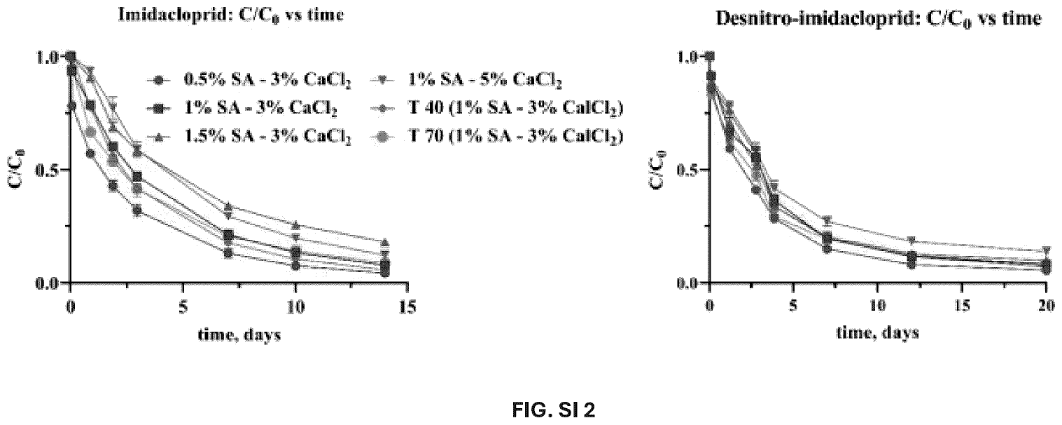

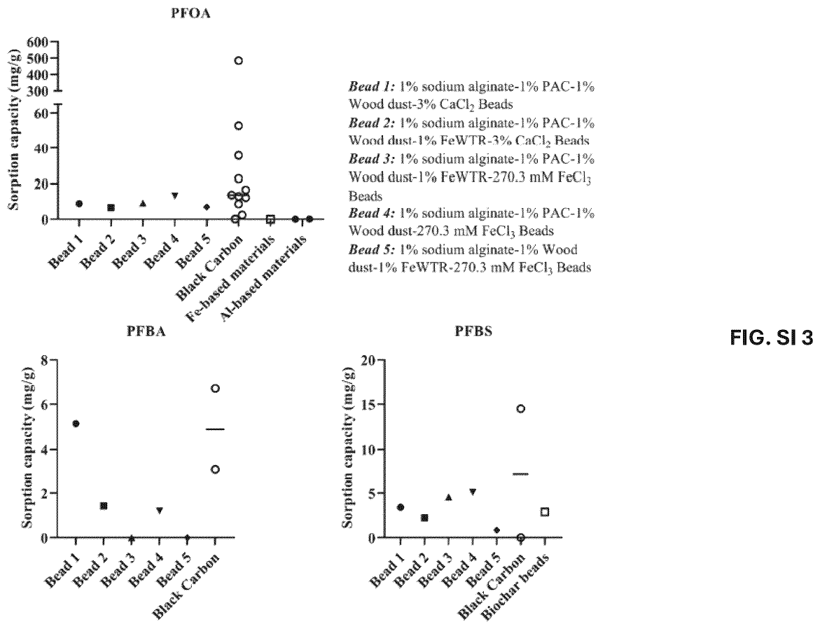

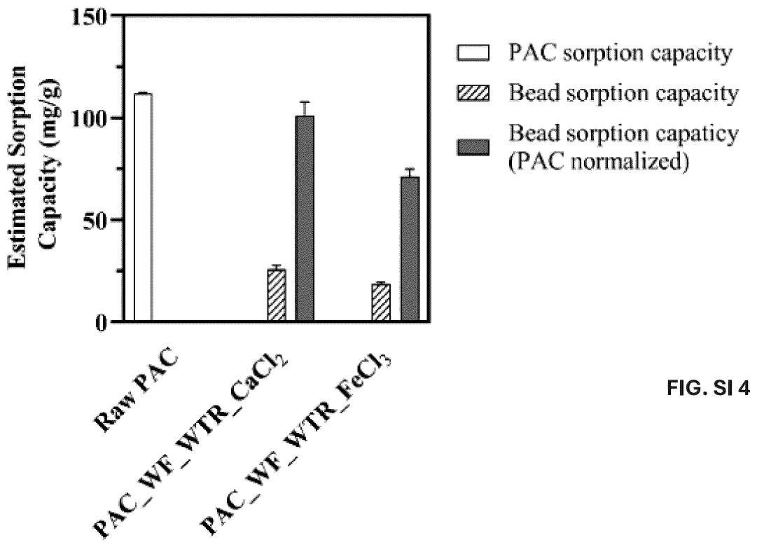

BioSorp Beads containing live microorganisms (biotic beads) completely removed all nitrate from the synthetic stormwater within a week (p=0.9959), whereas only around 30% nitrate removal was achieved via the abiotic control beads (maximum sorption capacity was reached) (FIG. 9). This demonstrates the phenomenon of coupled nitrate sorption and biological denitrification in biotic bead systems. Two carbon sources were encapsulated in the beads (LB broth base and wood flour) to increase microbial viability and activity. Addition of the supplemental carbon source (glucose, C/N=6) in the experimental system will enhance microbial denitrification. Due to the presence of anion exchange resin (AER) in the beads, stormwater nitrate will be sorbed during the bioretention infiltration events. Because nitrate (attached onto the AER) and the denitrifying microorganisms would be in close local proximity, denitrification process will remove stormwater nitrate during antecedent dry periods and renew the sorption capacity of the spent resin. Indeed, nitrate was reported to remain bio-available after being sorbed onto AER and bio-regeneration via denitrification was utilized to renew nitrate sorption capacity of the spent AER.

Along with two anaerobic batch tests (with the 5% AER-CaCl₂) beads and the proposed beads), there was also a batch test where the experiment was started in bulk aerobic conditions (with the beads) and the experiment vials were shaken (120 rpm) on platform shaker table (nitrogen was not flushed in the vials; vial openings were not sealed with rubber stoppers; vial openings were slightly covered with foil paper). Nitrate removal is not normally expected under aerobic conditions, even though aerobic conditions are common in stormwater infiltration systems. Even in initially bulk aerobic conditions where nitrate removal would not be expected, encapsulated denitrifiers remained viable and continued to biodegrade nitrate. Furthermore, the whole batch systems became anaerobic in two days (likely due to aerobic respiration by some of the encapsulated microbes). This demonstrates that the presence of facultative anaerobes (can sustain and thrive in periodic exposure to oxygen, as well as anoxic conditions) in the proposed beads and demonstrates one of many benefits of enriching and encapsulating mixed culture microbes instead of pure culture microbes. Additionally, encapsulation can maintain local anoxic zones to facilitate denitrification reactions. Because most bioretention cells (more than 80%) do not contain saturated underdrain layers (likely due to the high construction costs), low-cost amendments to existing and new bioretention cells are necessary to effectively manage stormwater nitrate pollution. As such, the BioSorp Beads can be utilized to amend existing and new bioretention cells to achieve sustained nitrate removal via rapid sorption during infiltration and denitrification during antecedent dry periods (in existing bioretention cells—by removing a little bit of bioretention top soil layer to deposit the beads and covering with soil, mulch, and vegetation; in new constructions—by installing the beads in the top soil layer and covering with mulch and vegetation).

### BIBLIOGRAPHY

1 Y.-Y. Yang and G. S. Toor, Sources and mechanisms of nitrate and orthophosphate transport in urban stormwater runoff from residential catchments, Water Res, 2017, 112, 176-184.
2 S. Wang, Y. Ma, X. Zhang and Z. Shen, Transport and sources of nitrogen in stormwater runoff at the urban catchment scale, Science of The Total Environment, 2022, 806, 150281.
3 S. I. Promi, C. M. Gardner and A. K. Hohner, Biodegradability of unheated and laboratory heated dissolved organic matter, Environ. Sci.: Processes Impacts, 2024, 26, 1429-1439.
4 D. S. Tanmoy, J. C. Bezares-Cruz and G. H. LeFevre, The use of recycled materials in a biofilter to polish anammox wastewater treatment plant effluent, Chemosphere, 2022, 296, 134058.
5 D. M. Anderson, P. M. Glibert and J. M. Burkholder, Harmful algal blooms and eutrophication: Nutrient sources, composition, and consequences, Estuaries, 2002, 25, 704-726.
6 V. H. Smith, Eutrophication of freshwater and coastal marine ecosystems a global problem, Environmental Science and Pollution Research, 2003, 10, 126-139.
7 W. K. Dodds, W. W. Bouska, J. L. Eitzmann, T. J. Pilger, K. L. Pitts, A. J. Riley, J. T. Schloesser and D. J. Thornbrugh, Eutrophication of US freshwaters: analysis of potential economic damages, Environ Sci Technol, 2009, 43, 12-19.
8 U.S. Environmental Protection Agency, National Rivers and Streams Assessment 2018-2019 Technical Support Document. Version 1.1 EPA 841-R-22-005., 2024.
9 J. Zuo, P. Xiao, J. Heino, F. Tan, J. Soininen, H. Chen and J. Yang, Eutrophication increases the similarity of cyanobacterial community features in lakes and reservoirs, Water Res, 2024, 250, 120977.
10 P. Zhang, L. Chen, T. Yan, J. Liu and Z. Shen, Sources of nitrate-nitrogen in urban runoff over and during rainfall events with different grades, Science of The Total Environment, 2022, 808, 152069.
11 S. Wang, Y. Ma, X. Zhang, Y. Yu, X. Zhou and Z. Shen, Nitrogen transport and sources in urban stormwater with different rainfall characteristics, Science of The Total Environment, 2022, 837, 155902.
12 Y.-Y. Yang and M. G. Lusk, Nutrients in Urban Stormwater Runoff: Current State of the Science and Potential Mitigation Options, Curr Pollut Rep, 2018, 4, 112-127.
13 Z. Kong, Y. Song, M. Xu, Y. Yang, X. Wang, H. Ma, Y. Zhi, Z. Shao, L. Chen, Y. Yuan, F. Liu, Y. Xu, Q. Ni, S. Hu and H. Chai, Multi-media interaction improves the efficiency and stability of the bioretention system for stormwater runoff treatment, Water Res, 2024, 250, 121017.
14 G. H. LeFevre, M. D. Hendricks, M. E. Carrasquillo, L. E. McPhillips, B. K. Winfrey and J. R. Mihelcic, The Greatest Opportunity for Green Stormwater Infrastructure Is to Advance Environmental Justice, Environ Sci Technol, 2023, 57, 19088-19093.
15 H. Kim, E. A. Seagren and A. P. Davis, Engineered Bioretention for Removal of Nitrate from Stormwater Runoff, Water Environment Research, 2003, 75, 355-367.
16 C. M. Rodak, T. L. Moore, R. David, A. D. Jayakaran and J. R. Vogel, Urban stormwater characterization, control, and treatment, Water Environment Research, 2019, 91, 1034-1060.
17 L. Mutzner, K. Zhang, R. G. Luthy, H. P. H. Arp and S. Spahr, Urban stormwater capture for water supply: look out for persistent, mobile and toxic substances, Environ. Sci.: Water Res. Technol., 2023, 9, 3094-3102.
18 J. R. Masoner, D. W. Kolpin, I. M. Cozzarelli, L. B. Barber, D. S. Burden, W. T. Foreman, K. J. Forshay, E. T. Furlong, J. F. Groves, M. L. Hladik, M. E. Hopton, J. B. Jaeschke, S. H. Keefe, D. P. Krabbenhoft, R. Lowrance, K. M. Romanok, D. L. Rus, W. R. Selbig, B. H. Williams and P. M. Bradley, Urban Stormwater: An Overlooked Pathway of Extensive Mixed Contaminants to Surface and Groundwaters in the United States, Environ Sci Technol, 2019, 53, 10070-10081.
19 T. F. M. Rodgers, L. Wu, X. Gu, S. Spraakman, E. Passeport and M. L. Diamond, Stormwater Bioretention Cells Are Not an Effective Treatment for Persistent and Mobile Organic Compounds (PMOCs), Environ Sci Technol, 2022, 56, 6349-6359.
20 T. F. M. Rodgers, S. Spraakman, Y. Wang, C. Johannessen, R. C. Scholes and A. Giang, Bioretention Design Modifications Increase the Simulated Capture of Hydrophobic and Hydrophilic Trace Organic Compounds, Environ Sci Technol, 2024, 58, 5500-5511.
21 J. E. Grebel, J. A. Charbonnet and D. L. Sedlak, Oxidation of organic contaminants by manganese oxide geomedia for passive urban stormwater treatment systems, Water Res, 2016, 88, 481-491.
22 B. K. Biswal, K. Vijayaraghavan, D. L. Tsen-Tieng and R. Balasubramanian, Biochar-based bioretention systems for removal of chemical and microbial pollutants from stormwater: A critical review, J Hazard Mater, 2022, 422, 126886.
23 J. Sarazen, S. Hurley and J. Faulkner, Nitrogen and phosphorus removal in a bioretention cell experiment receiving agricultural runoff from a dairy farm production area during third and fourth years of operation, J Environ Qual, 2023, 52, 149-160.
24 A. P. Davis, M. Shokouhian, H. Sharma and C. Minami, Water Quality Improvement through Bioretention Media: Nitrogen and Phosphorus Removal, Water Environment Research, 2006, 78, 284-293.
25 S. Wang, X. Lin, H. Yu, Z. Wang, H. Xia, J. An and G. Fan, Nitrogen removal from urban stormwater runoff by stepped bioretention systems, Ecol Eng, 2017, 106, 340-348.
26 Y. Li, Y. Zhang, H. Yu, Y. Han and J. Zuo, Enhancing nitrate removal from urban stormwater in an inverted bioretention system, Ecol Eng, 2021, 170, 106315.
27 J. Wu, X. Cao, J. Zhao, Y. Dai, N. Cui, Z. Li and S. Cheng, Performance of biofilter with a saturated zone for urban stormwater runoff pollution control: Influence of vegetation type and saturation time, Ecol Eng, 2017, 105, 355-361.
28 K. He, H. Qin, F. Wang, W. Ding and Y. Yin, Importance of the Submerged Zone during Dry Periods to Nitrogen Removal in a Bioretention System, Water (Basel), DOI: 10.3390/w12030876.
29 S. Yu, K. He, C. Xia and H. Qin, Modeling the effect of the submerged zone on nitrogen removal efficiency of a bioretention system under dry-wet alterations, J Hydrol (Amst), 2023, 623, 129788.
30 P. I. James, I. Sara and D. A. P, Enhanced Denitrification in Bioretention Using Woodchips as an Organic Carbon Source, J Sustain Water Built Environ, 2015, 1, 04015004.
31 Y. Zinger, G.-T. Blecken, T. D. Fletcher, M. Viklander and A. Deletić, Optimising nitrogen removal in existing stormwater biofilters: Benefits and tradeoffs of a retrofitted saturated zone, Ecol Eng, 2013, 51, 75-82.
32 T. Huang, J. Sage, D. Techer and M.-C. Gromaire, Hydrological performance of bioretention in field experiments and models: A review from the perspective of design characteristics and local contexts, Science of The Total Environment, 2025, 965, 178684.
33 W. Ding, H. Qin, S. Yu and S.-L. Yu, The overall and phased nitrogen leaching from a field bioretention during rainfall runoff events, Ecol Eng, 2022, 179, 106624.
34 Y. Zhang, A. Skorobogatov, J. He, C. Valeo, A. Chu, B. van Duin and L. van Duin, Mitigation of nutrient leaching from bioretention systems using amendments, J Hydrol (Amst), 2023, 618, 129182.
35 Y. Yao, B. Gao, M. Zhang, M. Inyang and A. R. Zimmerman, Effect of biochar amendment on sorption and leaching of nitrate, ammonium, and phosphate in a sandy soil, Chemosphere, 2012, 89, 1467-1471.
36 D. S. Tanmoy and G. H. LeFevre, Development of composite alginate bead media with encapsulated sorptive materials and microorganisms to bioaugment green stormwater infrastructure, Environ Sci (Camb), 2024, 10, 1890-1907.
37 D. S. Tanmoy and G. H. LeFevre, Sorption and biodegradation of stormwater trace organic contaminants via composite alginate bead geomedia with encapsulated microorganisms, Environ Sci (Camb), 2024, 10, 3339-3357.
38 M. V. Tuttolomondo, G. S. Alvarez, M. F. Desimone and L. E. Diaz, Removal of azo dyes from water by sol-gel immobilized Pseudomonas sp., J Environ Chem Eng, 2014, 2, 131-136.
39 E. A. Wiener and G. H. LeFevre, White Rot Fungi Produce Novel Tire Wear Compound Metabolites and Reveal Underappreciated Amino Acid Conjugation Pathways, Environ Sci Technol Lett, 2022, 9, 391-399.
40 M. F. Ahmer and M. K. Uddin, Structure properties and industrial applications of anion exchange resins for the removal of electroactive nitrate ions from contaminated water, RSC Adv., 2024, 14, 33629-33648.
41 N. Öztürk and T. E. Bektaş, Nitrate removal from aqueous solution by adsorption onto various materials, J Hazard Mater, 2004, 112, 155-162.
42 B. A. Ulrich, E. A. Im, D. Werner and C. P. Higgins, Biochar and Activated Carbon for Enhanced Trace Organic Contaminant Retention in Stormwater Infiltration Systems, Environ Sci Technol, 2015, 49, 6222-6230.
43 N. Ashoori, M. Teixido, S. Spahr, G. H. LeFevre, D. L. Sedlak and R. G. Luthy, Evaluation of pilot-scale biochar-amended woodchip bioreactors to remove nitrate, metals, and trace organic contaminants from urban stormwater runoff, Water Res, 2019, 154, 1-11.
44 Z. Kong, H. Wang, H. Wang, S. Man and Q. Yan, Magnetite-mediated shifts in denitrifying consortia in bioelectrochemical system: Insights into species selection and metabolic dynamics, Water Res, 2024, 262, 122132.
45 Y. Bai, S. Wang, A. Zhussupbekova, I. V Shvets, P.-H. Lee and X. Zhan, High-rate iron sulfide and sulfur-coupled autotrophic denitrification system: Nutrients removal performance and microbial characterization, Water Res, 2023, 231, 119619.
46 X. Meng, D. A. Vaccari, J. Zhang, A. Fiume and X. Meng, Bioregeneration of Spent Anion Exchange Resin for Treatment of Nitrate in Water, Environ Sci Technol, 2014, 48, 1541-1548.
47 A. C. André, L. Debande and B. S. Marteyn, The selective advantage of facultative anaerobes relies on their unique ability to cope with changing oxygen levels during infection, Cell Microbiol, 2021, 23, e13338.
48 H. A. Ahmad, S.-Q. Ni, S. Ahmad, J. Zhang, M. Ali, H. H. Ngo, W. Guo, Z. Tan and Q. Wang, Gel immobilization: A strategy to improve the performance of anaerobic ammonium oxidation (anammox) bacteria for nitrogen-rich wastewater treatment, Bioresour Technol, 2020, 313, 123642.
49 Z. Wang, S. Ishii and P. J. Novak, Encapsulating microorganisms to enhance biological nitrogen removal in wastewater: recent advancements and future opportunities, Environ Sci (Camb), 2021, 7, 1402-1416.

All publications, patents, and patent applications, Genbank sequences, websites and other published materials referred to throughout the disclosure herein are herein incorporated by reference to the same extent as if each individual publication, patent, or patent application, Genbank sequences, websites and other published materials was specifically and individually indicated to be incorporated by reference. In the event that the definition of a term incorporated by reference conflicts with a term defined herein, this specification shall control.

What is claimed is:

1. A biosorption bead comprising a biopolymer matrix formed from an alginate hydrogel, a sorbent material dispersed in said matrix, a growth substrate incorporated into said matrix and a biodegrading organism encapsulated into said matrix.

2. The biosorption bead of claim 1, wherein the sorbent is activated carbon, iron-based water treatment residual (FeWTR), powdered biochar, aluminum-based water treatment residuals, iron oxide coated sorbents, zinc oxide coated sorbents, manganese oxide coated sand or a combination thereof.

3. The biosorption bead of claim 2, wherein the activated carbon is powdered activated carbon (PAC).

4. The biosorption bead of claim 1, wherein the growth substrate is wood flour (WF), mulch/wood chips, corn and/or corn cob, shredded straw, grass, newspaper, cotton, rice husk, chlorella or combination thereof.

5. The biosorption bead of claim 1, further comprising an electron shuttle.

6. The biosorption bead of claim 5, wherein the electron shuttle comprises anthraquinone-2,6-disulfonate (AQDS).

7. The biosorption bead of claim 1, further comprising anion exchange resin (AER).

8. The biosorption bead of claim 1, wherein the biodegrading organism is a fungus.

9. The biosorption bead of claim 8, wherein the fungus is a white-rot-fungi (WRF).

10. The biosorption bead of claim 9, wherein the WRF is a Trametes versicolor, P. ostreatus or a combination thereof.

11. The biosorption bead of claim 1, wherein the biodegrading organism is a bacterium.

12. The biosorption bead of claim 11, wherein the bacterium is a denitrifying bacterium or a nitrifying bacterium.

13. The biosorption bead of claim 1, wherein the biodegrading organism is stable for at least three months when stored at room temperature.

14. The biosorption bead of claim 1, wherein the alginate hydrogel is a cation alginate hydrogel.

15. The biosorption bead of claim 14, wherein the cation alginate hydrogel is sodium alginate hydrogel.

16. The biosorption bead of claim 1, wherein the alginate hydrogel is crosslinked.

17. The biosorption bead of claim 1, wherein the alginate hydrogel is crosslinked with calcium ions or ferric ions.

18. The biosorption bead of claim 1, wherein the alginate hydrogel is crosslinked with CaCl₂) or FeCl₃.

19. The biosorption bead of claim 1, further comprising one or more micronutrients/vitamins.

20. The biosorption bead of claim 19, wherein the one or more micronutrients/vitamins are vitamin B12.

21. The biosorption bead of claim 1, wherein the bead is configured to sorb at least about 20 mg of dissolved contaminants per gram of bead.

22. A method for coupling sorption of a dissolved contaminate from a liquid with subsequent biodegradation of said contaminate comprising contacting said contaminated liquid with the biosorption bead(s) of claim 1.

23. A method to decontaminate a liquid comprising contacting said contaminated liquid with the biosorption bead(s) of claim 1, wherein at least one dissolved contaminate is removed from the liquid by the biosorption bead.

24. The method of claim 22, wherein the organism biodegrades the contaminate.

25. The method of claim 21, wherein the biodegradation of the contaminate renews the sorption capacity of the biosorption bead.

26. The method of claim 24, wherein the contaminate is a trace organic contaminant (TOrC).

27. The method of claim 22, wherein the contaminate is phosphate or nitrogen.

28. The method of claim 22, wherein the contaminated liquid is runoff, wastewater, grey water, and/or stormwater.
